SEQUENCE LISTING (35 sequences)

SEQ ID NO: 1 - DNA, 669 bp, Mus musculus
  source 1..669: mol_type = genomic DNA
atgtcacagaccaaaacagcgaaggttcgtgtgaccctcttcttcatcctggtgggcggggtgctcgccatggtggccgtggtgactgaccactgggccgtgctgagtccacacctggagcaccacaatgaaacgtgcgaggcagcccactttggcctctggaggatctgcaccgctcgggttgccgtgcacaacaaagacaagagttgtgagcacgtcacaccatcaggggaaaagaactgctcctacttcaggcacttcaacccaggggagagctcggaaatctttgaattcaccactcaaaaggagtacagcatctcagcagcggccattgccatcttcagcctcggcttcatcattgtgggttccatctgcgcatttctgtccttcgggaataagcgtgattacctgctgaggccagcatccatgttttatgccttcgcagggctctgcctcatcgtctccgtggaggtcatgaggcagtcggtgaagcgtatgattgacagcgaggacacggtctggatagagcactactattcgtggtctttcgcctgtgcatgtgccgcgttcatcttgctcttcctcggtgggctgttcctcctgctcttctccctgcctcggatgcctcagaacccctgggaatcctgcatggacgctgagccagagcac

SEQ ID NO: 2 - protein, 223 aa, Mus musculus
MSQTKTAKVRVTLFFILVGGVLAMVAVVTDHWAVLSPHLEHHNETCEAAHFGLWRICTARVAVHNKDKSCEHVTPSGEKNCSYFRHFNPGESSEIFEFTTQKEYSISAAAIAIFSLGFIIVGSICAFLSFGNKRDYLLRPASMFYAFAGLCLIVSVEVMRQSVKRMIDSEDTVWIEHYYSWSFACACAAFILLFLGGLFLLLFSLPRMPQNPWESCMDAEPEH

SEQ ID NO: 3 - DNA, 666 bp, Homo sapiens
  source 1..666: mol_type = genomic DNA
atgtcccagaccaaaatgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcac

SEQ ID NO: 4 - protein, 222 aa, Homo sapiens
MSQTKMLKVRVTLFCILAGIVLAMTAVVTDHWAVLSPHMEHHNTTCEAAHFGLWRICTKRIPMDDSKTCGPITLPGEKNCSYFRHFNPGESSEIFEFTTQKEYSISAAAIAIFSLGFIILGSLCVLLSLGKKRDYLLRPASMFYAFAGLCILVSVEVMRQSVKRMIDSEDTVWIEYYYSWSFACACAAFILLFLGGLALLLFSLPRMPRNPWESCMDAEPEH

SEQ ID NO: 5 - DNA, 651 bp, Homo sapiens
  source 1..651: mol_type = genomic DNA
atgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcac

SEQ ID NO: 6 - DNA, 666 bp, synthetic construct
  misc_feature 1..666: note = mouse/human CACNG1 cds
  source 1..666: mol_type = other DNA
atgtcacagaccaaaatgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcac

SEQ ID NO: 7 - DNA, 30 bp, Homo sapiens
  source 1..30: mol_type = genomic DNA
atgtcccagaccaaaatgctgaaggtccgc

SEQ ID NO: 8 - protein, 10 aa, Homo sapiens
MSQTKMLKVR

SEQ ID NO: 9 - DNA, 57 bp, Homo sapiens
  source 1..57: mol_type = genomic DNA
gtgaccctcttctgcatcctggcaggcatcgtgctggccatgacagccgtggtaacc

SEQ ID NO: 10 - protein, 19 aa, Homo sapiens
VTLFCILAGIVLAMTAVVT

SEQ ID NO: 11 - DNA, 237 bp, Homo sapiens
  source 1..237: mol_type = genomic DNA
gaccactgggctgtgctgagcccccacatggagcaccacaacactacctgcgaggcggcccacttcggcctctggcggatttgtaccaagcgcatccccatggacgacagcaagacctgcgggcccatcaccctgcccggggagaagaactgttcctacttcaggcattttaaccccggcgagagctcggagatcttcgaattcaccactcagaaggagtacagcatctcggcagcc

SEQ ID NO: 12 - protein, 79 aa, Homo sapiens
DHWAVLSPHMEHHNTTCEAAHFGLWRICTKRIPMDDSKTCGPITLPGEKNCSYFRHFNPGESSEIFEFTTQKEYSISAA

SEQ ID NO: 13 - DNA, 63 bp, Homo sapiens
  source 1..63: mol_type = genomic DNA
gccatcgccatcttcagccttggcttcatcatcctgggcagcctctgtgtcctcctgtccctc

SEQ ID NO: 14 - protein, 21 aa, Homo sapiens
AIAIFSLGFIILGSLCVLLSL

SEQ ID NO: 15 - DNA, 15 bp, Homo sapiens
  source 1..15: mol_type = genomic DNA
gggaagaagagggac

SEQ ID NO: 16 - protein, 5 aa, Homo sapiens
GKKRD

SEQ ID NO: 17 - DNA, 63 bp, Homo sapiens
  source 1..63: mol_type = genomic DNA
tatctgctgcgacccgcgtccatgttctatgcctttgcaggtctctgcatcctcgtctcggtg

SEQ ID NO: 18 - protein, 21 aa, Homo sapiens
YLLRPASMFYAFAGLCILVSV

SEQ ID NO: 19 - DNA, 72 bp, Homo sapiens
  source 1..72: mol_type = genomic DNA
gaggtcatgcggcagtcggtgaagcgcatgattgacagtgaggacaccgtctggatcgagtactattactcc

SEQ ID NO: 20 - protein, 24 aa, Homo sapiens
EVMRQSVKRMIDSEDTVWIEYYYS

SEQ ID NO: 21 - DNA, 75 bp, Homo sapiens
  source 1..75: mol_type = genomic DNA
tggtcctttgcctgcgcctgtgccgccttcatcctcctctttctcggcggtctcgccctcctgctgttctccctg

SEQ ID NO: 22 - protein, 25 aa, Homo sapiens
WSFACACAAFILLFLGGLALLLFSL

SEQ ID NO: 23 - DNA, 54 bp, Homo sapiens
  source 1..54: mol_type = genomic DNA
cctcgaatgccccggaacccatgggagtcctgcatggatgctgagcccgagcac

SEQ ID NO: 24 - protein, 18 aa, Homo sapiens
PRMPRNPWESCMDAEPEH

SEQ ID NO: 25 - DNA, 52 bp, Mus musculus
  source 1..52: mol_type = other DNA
gccccctgcccagagcacccacacctcaacgaccgccatgtcacagaccaaa

SEQ ID NO: 26 - DNA, 57 bp, Mus musculus
  source 1..57: mol_type = other DNA
tgctcctctacaaacagtgcacatggacacaaacgcagacctgcccagggtcagagg

SEQ ID NO: 27 - DNA, 17397 bp, synthetic construct
  misc_feature 1..17397: note = 7450 allele
  source 1..17397: mol_type = other DNA
gccccctgcccagagcacccacacctcaacgaccgccatgtcacagaccaaaatgctgaaggtccgcgtgaccctcttctgcatcctggcaggcatcgtgctggccatgacagccgtggtaaccgaccactgggctgtgctgagcccccacatggagcaccacaacactacctgcgaggcggcccacttcggcctctggcggatttgtaccaagcgcatccccatggacgacagcaagacctgcgggcccatcaccctgcccgggggtaacgtacccaccctccgtcccgatccccacctcctgctcttccccgtcatccccctggcaaagttgcccttgcgaaggaaggcaggtttctctgcctagaaatagggcagccgcccagccttttctgcccagggaagaagatatgaattgcatgtgtggtgctggtggctggggctgggttgtaagacgagggcgtttgtcccagctatttctgtctggctggaccttggggctttttgcccagagcagagagctgggcaggctcaagaaaggaactgcagctctaccagcccagggacttctgggccccacctgctcactctctccctgcaaagaaaagctaggtggctctgtgtcctcaggctagggggacccatggggctggcaggtttgttcctggcctcaaggcaggagaggagagagcataggcgatggaatttgcagggtcctgtgcagaatgaaaatgcagggttccttgctccaaattaccaaggatttcaaggcagtgacagcagcacatgaaaccacacatgggcccttgtgggctctgggcctggagcagctgcacaggttgaatgtctgtgaagcaggacatggctgtaggctgtaggtgagccacctgcccccaccttctttttttttttttttttttttttgagacaactttgtcgctcaggctggagtgcagtggcatgatcttggctccctgcaacttctgcctcctggactcaagcgattctcctgcctcagcttccccagtagctgggattacaggcatgcaccaccacacccatctaatttttgtactttcagtagagacggggtttcaccatgttggtcaggctggtctcgaactcctgacctcaggtgatccacctgccttggcctcccagagtgctgggattacagacgtgagccaccatgcccgacctccacctgtccttttgtcaggacttcagtcctctatgcatcctgtcttagctcagaccctgtcagagttacaaagcatttcctcaccctgtaatttaagcgtattcctcgatttcctaccgtgtgaccgaaccacacgttgaaggtaaggtccttttcaggagtacctggtagtcgcgtgactagcctctattctgcaaatgcagaggcttctggattcttagcaaatacagcactttagaggaaagagtgctcatggagaccaggcagccagggatctaatcgccattgcccactaccctcaaatgaccatccagggaggacaaagctcatctcagcctctgcttcttcctctataaaatatgggactgactctcagcaacgtgagatcccttcctcggcttacagacgcccatccacagtggcagtggagcagagtgatgtcagtgtgatgtcccacctggttttcaggattatttcctcttttatcagactaggtgttccttgaaacctgtccttcaaggacactagggctaagtccccaaagatcttgcagtctgcttgtgaggcaaagagcacagaccccatcctcccttgtctctgctccctcttccttccctcctgcttgctccagagatcccctcccttgttcagccccactggaggcccagaagactcataagtgcccagctgttgtagaaacaccaagtcccagggctgagagtgacaggtgtggccatgtgcttgcagggtatcgcatagcccggatgcccatgccccttggccactgctgagcacacccataaacaggaacagcctccagccaactcccctgggcagggcagggcagggcagggcagggcaaggcagcttcttcagggaccacacatggggacgtgctgttacagtcctgttcacccgtccttggaaccatctgccttttaaagtcagagcagaagactgggcagtgagcagaatccacacaaggaagagacaggctggtgagaggtgactggaccaattgcagggggccaacaggcattgcttggccttgagcctgacaggatctacctctttgccttcttagcctaaccagctatgaaccacacctcccacacttgtgacactgagcactacctttcccccagtaatcttccaaaatatgttcctgcctttttccctctcttttctctgcaaatggacatctttctccttccccatctgcccttcttgtgctccctgggaccactcttctctggagggaggaagtgggttcatatgtggcccgtcatctattattaacaacaataataataatggcacatacatgtagccctgtgtgccaaatattgttgtaaacgctttacatatattaactttgaaagacatcaggaaaaaaaatgatagaaaaagaacctccaaaaataatcttctccataatagtaatgagaaggctggcagaaatggtcagaatcaacattttcagaactctggaaattaaccaaagtcttgcagcaatccagcaagaaaaatgggtgaatctcaggaggaccagtgagttctgtggtgttttaacttgccttattcctgtttccctttcccagctctgtggtaaccttgcaaaccaacattccgtaatcacagtgaaaatcagcatcccagtagcctctggaggggagagaatggggttggatcaaaggcccattcccagagagttgtcattatttgacctgtctggtgattccctgaaaacccccacttgagaagctgtttctgtttgacctgactctgagctcatccagtgcaaatagtctttctccctggggatgtttgttgaaaataattagagatgattgattaacatcatggctgtctgaggcagtagataacagttagggaaaacaacaggctaaccaaaaaactggagactgaaatgtccataagttgctttgaaaagctccaacatattcctgggaatctaggaggccacatgcatgcacaatgctatatgcatgctcaggaaagacctgagaaggccctaagcactctctgccttatcttgagactctgttcaagcaggaagtgaaggctaaggcagagatgtgggtggcttagctgagcatgaaagtcatatgcacagagcccctcagtaaagactgggagaggcattgttgccaagcgttttaggaaatttataattggtcactaagctaattgaacagggatctcagtgatcacacatgacaaaaaacacatactttaccatcttagttcaaaaaagtcactaaacaaacaacgggaacaaatcttcaagaagggggaacaactgatttccagagttgccacattatattgtttaaaaaatactggctgggtgtggtggctcactcctgtattcccagcacttttggaggccaagtgagaggactgcttgaggccaggggtttgagatcaacctgggcaatatagcaagaccctgtccctaccaaaaaaaaaaaaaaaaacaaaaaaaaaaccccacaaaatatccaggagtggtggcatgtgcctgtaattctagctactcccaaggcttaggtgagggaattgtttgatcccaggagttagagactacagtgaactatgatcatgccactgcactccagcctgggtgacagagcaagactctgtctctaaataaataaatacatacataaataatacaagacacacaaagaactaaaatgataaggggcatacatggggcagggagaagtcaatagatctgtaaggaagccagatattggacttactagacaaaaactttaaatcagctatagtaaatatatccaaagaaaaaacaatcaaaacaattaaagatgaaaatgatgcctcatgaaatagaaactatcaattaagacacagaaattatttttaaggacaaaatagaaattctggagttgaaaagtacaataactaaaatgagaaattcactagaggggctcaatggcatatctgagcaggcaaaagaatcagtgacctttaagataagtcagttgagattagccagtctgaggaacagacagataaaaagaataaagaaaaatgacgagagcctcggagacttatgggacaccatcaagtttatgtaaagggagtcccagaaggagagggaaagagagaaaggagcaaaaaaggtatgtgaaaaataatgtccaaaagcttcccaaattggatgaaaaacgttaaaaatcgacaaatttatgaagcttaatgaactccaggtagaataaactctaagggatccacatctagacacatgataatcaaactgttgaaagatagagacagagaatcttaaaaacggaaagaaacaactcaccacatatgaggaatccttaataagatgaacagccaatttttccccaaatctatggaggccagaaggtggtgggatgacatacatatattagctcacctaatcctcacagaaacctccgtgaaggccatgctattatcatcatcccaatcttacagataaagaaaatcaaccactgagagttgagatatttgacaaggtaacacaggtgatgagcagagcagccaggactcacactcagaccctaacattcaccgttttagtcaatacattaactactttacaacaacccagtgagtgggttcttattctacaggtggttcttattctacaggtgggtctcattctacaggtggttctcattctacaggtggttcctattctacaggtggttctcattctacaggtgggtctcattctacaggtaggaacagggaggctgagcatagccttccccaggctaaccagctaacaaatagaggttctccactcaagtcttgaatccagagcctcctttccctccagttccttctcacaccctctattgtgcatgaaaacacagctcctaaaagctctgacccaagtgaacacaaggagtccagcttttgacatggcatagtttgagggcagctggaccagaagctggtgcagtggagcagacttctcaaggtcccacttagtcttaaaatgttggaggctcagtgggcaagagttgtctaatttttccaaattagacagtcttagactccaaacagtcttagaaggacttccatgtacaagatcaacatccaagaccaattccaatgcctccagaatgagagtaggagcctctcccaaaggagagactatgtgttctgaaagtccaaatgaacccgcaaagaggccatcccctgcccttgggctgggtgaagtgctgcttctttcagactctgcctctttagggcaaggaggcaggttgccccagggcccactttgcctagtgccccattttccctactggtccatcttctctcagtgcgggaactgggattgtataaaagagcatgatactactgaccccaggaatttcatgggctcacttcaccacccacagaagtccatgccagaggcctaggccaaagccaggcttctcccatggaagagcagtgctctgaagctctagggtcaggtcttggagatttccagcccattttataaaattcttcaatcagagatgtggaaggagacacagcccagagaaaccactgttgggagctgatagaacttcttttaccaaaaggctaaaaacaacactgacatcaatgcaagttagaggcttccttgcacccctaaagtaccagtttctttggttttggacaaatccacaaagtatctttgaatatatgaaagtctccatggacagtcacttggcacactgatacattctcagtgataacccaaggcatccactaatcctgccagctgttccaactatcaaagaaagatccctattaactcaagttcagcttggagctaaatgtggcttctgctctaattctttgaaactggcacactacacatggttctgcacagttactgatgcccttaaccagaacactgggttagccagactattccatcccccagtcaagtctgagagctgagttcaccatatttttaataaataacccatttaatatttttaaaccgctgagtcttacagaaataaggagattaaaatcagcataagccatctgcagagacaaaggagaatggcacgacctcagagttcctaacaccccagatcatcatcctggcattggaatctttgctgacttagtcggaagcaaagctgcaatccctgagctttgtgtaagtgtgatggaagccagggcagcggggcagagaggggcatcccagaggcagaggcaggaagactgagagtctgcacaggaccgtcacggtccctctcctcttgacccatgactaagatgggtccacctcggatgtccttcctctttacgttgccctggatgctcagtttcccaaataatatccacctagacccaggaacagggcagggagggaaggggctcatagtagggtctgtccatctgaacaggaccctgcaccctcttgagtaacatgggcagatgcctggcctgatttgtcctaaatgaggcctgggtctgacgtgcccccacccctacaggggccattagagaccatgaaagggtttatggtctccaatgggcagccacatagcccaggagtggatgtttctctcaggttcagggcaggagacaccggtctttttcacatgctaagcccaagaaaggggagaggtcagcccatcagtcctcaggcccccagttcagccatgtaagagtgaagctgaggcttcctgttaccctgagaaggaatcctacccactggaggccccctccctccatttgaggacacttaatggagaagagggaggaggaacactgtacctggaccaccaaaagaaaagcatccccccagaagctcacaacctggctcctgcagacacgtagcctacccctgtgtctgagttaagatcatttctatccaagcagccagcagccaggcggagcctggcgtttatctggtggggatttggtgagccaaatgctacctgtgtgagagtcacctcagcaacccaccaggtgcaaacagctgtgcccccagcaaggcactccatggccctcatctcagggccctccgctgggaaggattttaacttatggcttttgagcagtcagggaggcagctgaccatgaaatacacttgaattcagtcaattctgacaactgaaattggcccttcaagaatgaaacagaaacagaaatgtttctttgtccagagggagacacatggagacttatgacatagcatgggccattttgaagcagtgattcgagagatctacagccaataatctaacaaatagaagagatctgcattatgctttaaaatagcaccggatgcccagagagaatagcagagcaccaattctgacatcaccaccagcagtcagacaccccaggtaacgcggttcaaaaggaaagtgatttaaatatagaagcacacagagaaggctgtttatgccaggaatgggtactgctgctaaaccagatcccatctccaggggtcatttgcttcctcaacctcaggagaggcagcagccctggctctggggggaaggaggggcatggtatgagaccacaggcttaaggtgcaagaagaatgatttaggtccggttaaagaaagtcttcatcccaaagaaaagccttggtatcactgcaggtacatttccagaaagaactaggcatcaaccgccttatgagcatctaaatgaagcctggaagtgaagaaatgaattggcaaactagacaccatctcaactagagaaagagatgtttttctatttaagggtcactgatccacaggaaaagaaatcaatgaagggcctccacaaataaaaagcaaactaatttagttttggaaggaaacataatataaaataacccattcatccacttcctaaatcaagagcaaagaagataaaccgtatttagcaagaaataagactgtgctttttttttttttgagacagagtcccactctgtcacccaggctggagtgcagtgacacgacctcagctcactgcaacctctgcctcctgggttgaagcaattctcctgcctcagccttctaaagtgctgggattacaggtgtgagccaccacgcccggcctgtgctttgattttatagtatacctagctatgagagagagactgaaagagaaaaatggaaatgaaattatattttaaatcagcatgaatttccttgtgctgagcccctcccctctccctcatcaaattcagatttgcgttcctactgcccacagactttccatagcttttatgacaattatcaggtgggccaagcggctttgtgggggcaggtctcatctgggctgtctcgcaaagaccagggacccagtgaaaccacatgatggtcacattcagccctcgtaggggttccatctgcatccaggccatcccaatggccactcggaaaaagccctttgcagggcattttaaggggcctggagcagggttcccatcctggctgcacatcaggctctcccagaaagcttttgaaaaacactgtttcccaggtcccaccaatgaaccagggtgtgcctggattctgatgagaaacacagtcccaggtttggggttgatccagggtttctcaaccttgacactcttgatattttggcctggatcattccatgttgggaggacctgtcctgtgtgtcttgggatgttcagcaacgtctgtggcctctaccggctgggtgccaggtgtacccctctcaccaccaccataaccccaggcatcgccaagtgtcccctgggggggcagaatcggccccaacagagactgctgtttggttctttgttgattgtgcaccctgcacaccctgaaaaatggcttgagcctgggcctcggttaccctgtctgcctaatggggtgcagaacccacaggccgtctgccctgcagactcatccagcctcagctccctgaagacctcccagccctgcaggcaacctcattatcccactgagatgttgttctaagggtcaaagacagatgtttcagcccagattaaggatctggggagaagagaggctgccagagccccctgcatgccgagcagaatgacaccacgctctctgccaggcctctgtccttgctacgggttgctcccctcaactcacagtctgtctcctcctttgcagagaagaactgttcctacttcaggcattttaaccccggcgagagctcggagatcttcgaattcaccactcagaagggtgagcctgggtggaaaggggtctggggtgacaagaggggacttctgtgttgtgcaccactgggccagaaagtcattggcaaaagcactgacttcctcacgcctgatgagaagaagcctgtggtgcatttgaacaacagccttgtcagctccccgctccggaggccccaggcagccgccttccatctccagggcccggagcttccacacctcggggccagggagcgtttgcagaagcagcacctcctggtaggcccctacacaagggcagctgttgtgacgggaggtgtggatgtggaacaaatgctcagaagccccgtggtggctcttggtggcatgtgatagttagagtctgcagggacagtctgagtccctccctggcctagaaatccctccctgagtaccaggagggtgcggtgggctctgggagcttttcaggatccgcagagctcagacgcacacccattgtcgggagtacagacccgtgcctgcctgcgcccagacagctgcacacagggtgagtgggcagacacacagacacacactgacacacacagacacacacaacacagatacacacacacgacacacacaagacacagacgcacagagacactgacacacacgcatgatgacacacaaaatgacacacagtgacacagacacacactgatacacatgcatgatgacacacaaaatgacacacagtgacacacacagacacacactgacacacacgtacaatgacacagacacagagacacacactgacacacacgtacaatgacagacacagagacacacactgacacatatgcatgacacacaatgacacacacagacactgacacacacacagatacacacatacaatgacacacacaagacagacacagagacacacacttgacacacacacaatgacacacacagacactgacacacacactgtggtgcatggtgtggtccctaacgagccatgacaagagctcccatgctgaggccgcatgctgggtgtcccttgtgtttgcagagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtagactgggggatctgcctgagcgccggggccgggggaccatgtcgcgggggattctccgctccaccctcatgcccaccgcgagatttgggtgaggggcatttcccaggctccatccccatccaggggcaaacctggccaggccatcagcgactccaggtgggttggagggagacagagcttggcggtttgtccattcctctccacgactcaaccctcaaggccccaaagaaactgcccagagaatgattaacaaaagaaggaaagaaacgtggtcacctttctttctttttcttgggggatgaggaagagggtctcgctctgtcatccaggctggagtgcagtggcacaatcacagctcactgcagcctcgaactcctgggttcaagggttcctcccgccccagcctcctagtagctgggactacagacgtgcaccaccacacctggctaatttttcattgtattttaattttttgtagagacggagggctcatcactatgttgccaggctggtctcaaactcctggcctcaagcgatcctcctgcctcagcctcccaaagtgctgggattacaggtgtgagccaccacacctggccacatggttgcccttctgagggatgcaggaatagggtggtgggtggacaaatggatccttctgcaggttcccatctagaacctgccttgaggcagcttttcccccaaggcaaggtcaccgcctcctccatgcacacaggctgggatggggctggtggctacggcagacgcccctcggtccctgagcatgcctggctctgcccccaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcactaaccctcctgcggccctagcgaccctcaggcttcttccccaggaagcggggtcttggcctggaaccttccagagaggaggcgggagcaattttagccccaccctgctcccatctgcccccctgcaacagtcgcaggctgcttcctctctctgagttcctctgggctgccgcaggctcccctgggaatagagcaagacgtgagtcctaacctggccacagttgggggaggcagagccagcaggtggacaggtgtttgcaggggcccaacttcccctggagctcagaggtgtccccactgtaccagcctctgataagctgcctccagttgtcctttatgaacattgcagggacaacctgtgtttgccagctgggtgttccgtgtaaatagccagcctgtctctttctcggtgataaaacacaccctctctggtgagcccagcgtcccctccttggcttccaggagccctgggaagcatttttaactgggtagaatctgactgtggcttgaaataaaaagctctcagaaaactcatgctttcctggagccttgcatagaaatcgctcggattcccagtcacatcaggggctcccctgacgccgaatgagaaggaaaggccggtcacccgggtatgttgaggtcttttgtggggacaagagggcaacgcacgtggcttagaggctctgaattccaactcgtatctctaggccctgagattgttttcgggagaaactagaaaccctctgatgaagcaagggtgttaaaaataggaggggttatttctagagcctggcatgcaacgcacctggtgccaacacagcgctaaagcagccagccagtgtctgatttctgatgttggatttttcttgtgaagaacatcaggaagcagggcctcgagataacttcgtataatgtatgctatacgaagttatatgcatgccagtagcagcacccacgtccaccttctgtctagtaatgtccaacacctccctcagtccaaacactgctctgcatccatgtggctcccatttatacctgaagcacttgatggggcctcaatgttttactagagcccacccccctgcaactctgagaccctctggatttgtctgtcagtgcctcactggggcgttggataatttcttaaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggtaaatataaaatttttaagtgtataatgatgttaaactactgattctaattgtttgtgtattttaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggcggccggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctaggtaactataacggtcctaaggtagcgatgctcctctacaaacagtgcacatggacacaaacgcagacctgcccagggtcagagg

SEQ ID NO: 28 - DNA, 10741 bp, synthetic construct
  misc_feature 1..10741: note = 7451 allele
  source 1..10741: mol_type = other DNA
taaaaggtcaagttccctcagcagcattctctgagcagtctgaagatgtgtgcttttcacagttcaaatccatgtggctgtttcacccacctgcctggccttgggttatctatcaggacctagcctagaagcaggtgtgtggcacttaacacctaagctgagtgactaactgaacactcaagtggatgccatctttgtcacttcttgactgtgacacaagcaactcctgatgccaaagccctgcccacccctctcatgcccatatttggacatggtacaggtcctcactggccatggtctgtgaggtcctggtcctctttgacttcataattcctaggggccactagtatctataagaggaagagggtgctggctcccaggccacagcccacaaaattccacctgctcacaggttggctggctcgacccaggtggtgtcccctgctctgagccagctcccggccaagccagcaccatgggaacccccaagaagaagaggaaggtgcgtaccgatttaaattccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggtaaatataaaatttttaagtgtataatgatgttaaactactgattctaattgtttgtgtattttaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattaggcggccggccgctaatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatcccccgggatcatggcctccgcgccgggttttggcgcctcccgcgggcgcccccctcctcacggcgagcgctgccacgtcagacgaagggcgcagcgagcgtcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagtagcgggctgctgggctggccggggctttcgtggccgccgggccgctcggtgggacggaagcgtgtggagagaccgccaagggctgtagtctgggtccgcgagcaaggttgccctgaactgggggttggggggagcgcagcaaaatggcggctgttcccgagtcttgaatggaagacgcttgtgaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcggtttgtcgtctgttgcgggggcggcagttatggcggtgccgttgggcagtgcacccgtacctttgggagcgcgcgccctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaattgtccgctaaattctggccgtttttggcttttttgttagacgtgttgacaattaatcatcggcatagtatatcggcatagtataatacgacaaggtgaggaactaaaccatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagacctcgacctgcagcccctagataacttcgtataatgtatgctatacgaagttatgctaggtaactataacggtcctaaggtagcgatgctcctctacaaacagtgcacatggacacaaacgcagacctgcccagggtcagaggcacctcggatgtccttcctctttacgttgccctggatgctcagtttcccaaataatatccacctagacccaggaacagggcagggagggaaggggctcatagtagggtctgtccatctgaacaggaccctgcaccctcttgagtaacatgggcagatgcctggcctgatttgtcctaaatgaggcctgggtctgacgtgcccccacccctacaggggccattagagaccatgaaagggtttatggtctccaatgggcagccacatagcccaggagtggatgtttctctcaggttcagggcaggagacaccggtctttttcacatgctaagcccaagaaaggggagaggtcagcccatcagtcctcaggcccccagttcagccatgtaagagtgaagctgaggcttcctgttaccctgagaaggaatcctacccactggaggccccctccctccatttgaggacacttaatggagaagagggaggaggaacactgtacctggaccaccaaaagaaaagcatccccccagaagctcacaacctggctcctgcagacacgtagcctacccctgtgtctgagttaagatcatttctatccaagcagccagcagccaggcggagcctggcgtttatctggtggggatttggtgagccaaatgctacctgtgtgagagtcacctcagcaacccaccaggtgcaaacagctgtgcccccagcaaggcactccatggccctcatctcagggccctccgctgggaaggattttaacttatggcttttgagcagtcagggaggcagctgaccatgaaatacacttgaattcagtcaattctgacaactgaaattggcccttcaagaatgaaacagaaacagaaatgtttctttgtccagagggagacacatggagacttatgacatagcatgggccattttgaagcagtgattcgagagatctacagccaataatctaacaaatagaagagatctgcattatgctttaaaatagcaccggatgcccagagagaatagcagagcaccaattctgacatcaccaccagcagtcagacaccccaggtaacgcggttcaaaaggaaagtgatttaaatatagaagcacacagagaaggctgtttatgccaggaatgggtactgctgctaaaccagatcccatctccaggggtcatttgcttcctcaacctcaggagaggcagcagccctggctctggggggaaggaggggcatggtatgagaccacaggcttaaggtgcaagaagaatgatttaggtccggttaaagaaagtcttcatcccaaagaaaagccttggtatcactgcaggtacatttccagaaagaactaggcatcaaccgccttatgagcatctaaatgaagcctggaagtgaagaaatgaattggcaaactagacaccatctcaactagagaaagagatgtttttctatttaagggtcactgatccacaggaaaagaaatcaatgaagggcctccacaaataaaaagcaaactaatttagttttggaaggaaacataatataaaataacccattcatccacttcctaaatcaagagcaaagaagataaaccgtatttagcaagaaataagactgtgctttttttttttttgagacagagtcccactctgtcacccaggctggagtgcagtgacacgacctcagctcactgcaacctctgcctcctgggttgaagcaattctcctgcctcagccttctaaagtgctgggattacaggtgtgagccaccacgcccggcctgtgctttgattttatagtatacctagctatgagagagagactgaaagagaaaaatggaaatgaaattatattttaaatcagcatgaatttccttgtgctgagcccctcccctctccctcatcaaattcagatttgcgttcctactgcccacagactttccatagcttttatgacaattatcaggtgggccaagcggctttgtgggggcaggtctcatctgggctgtctcgcaaagaccagggacccagtgaaaccacatgatggtcacattcagccctcgtaggggttccatctgcatccaggccatcccaatggccactcggaaaaagccctttgcagggcattttaaggggcctggagcagggttcccatcctggctgcacatcaggctctcccagaaagcttttgaaaaacactgtttcccaggtcccaccaatgaaccagggtgtgcctggattctgatgagaaacacagtcccaggtttggggttgatccagggtttctcaaccttgacactcttgatattttggcctggatcattccatgttgggaggacctgtcctgtgtgtcttgggatgttcagcaacgtctgtggcctctaccggctgggtgccaggtgtacccctctcaccaccaccataaccccaggcatcgccaagtgtcccctgggggggcagaatcggccccaacagagactgctgtttggttctttgttgattgtgcaccctgcacaccctgaaaaatggcttgagcctgggcctcggttaccctgtctgcctaatggggtgcagaacccacaggccgtctgccctgcagactcatccagcctcagctccctgaagacctcccagccctgcaggcaacctcattatcccactgagatgttgttctaagggtcaaagacagatgtttcagcccagattaaggatctggggagaagagaggctgccagagccccctgcatgccgagcagaatgacaccacgctctctgccaggcctctgtccttgctacgggttgctcccctcaactcacagtctgtctcctcctttgcagagaagaactgttcctacttcaggcattttaaccccggcgagagctcggagatcttcgaattcaccactcagaagggtgagcctgggtggaaaggggtctggggtgacaagaggggacttctgtgttgtgcaccactgggccagaaagtcattggcaaaagcactgacttcctcacgcctgatgagaagaagcctgtggtgcatttgaacaacagccttgtcagctccccgctccggaggccccaggcagccgccttccatctccagggcccggagcttccacacctcggggccagggagcgtttgcagaagcagcacctcctggtaggcccctacacaagggcagctgttgtgacgggaggtgtggatgtggaacaaatgctcagaagccccgtggtggctcttggtggcatgtgatagttagagtctgcagggacagtctgagtccctccctggcctagaaatccctccctgagtaccaggagggtgcggtgggctctgggagcttttcaggatccgcagagctcagacgcacacccattgtcgggagtacagacccgtgcctgcctgcgcccagacagctgcacacagggtgagtgggcagacacacagacacacactgacacacacagacacacacaacacagatacacacacacgacacacacaagacacagacgcacagagacactgacacacacgcatgatgacacacaaaatgacacacagtgacacagacacacactgatacacatgcatgatgacacacaaaatgacacacagtgacacacacagacacacactgacacacacgtacaatgacacagacacagagacacacactgacacacacgtacaatgacagacacagagacacacactgacacatatgcatgacacacaatgacacacacagacactgacacacacacagatacacacatacaatgacacacacaagacagacacagagacacacacttgacacacacacaatgacacacacagacactgacacacacactgtggtgcatggtgtggtccctaacgagccatgacaagagctcccatgctgaggccgcatgctgggtgtcccttgtgtttgcagagtacagcatctcggcagccgccatcgccatcttcagccttggcttcatcatcctgggcagcctctgtgtcctcctgtccctcgggaagaagagggactatctgctgcgacccgcgtccatgttctatgcctttgcaggtagactgggggatctgcctgagcgccggggccgggggaccatgtcgcgggggattctccgctccaccctcatgcccaccgcgagatttgggtgaggggcatttcccaggctccatccccatccaggggcaaacctggccaggccatcagcgactccaggtgggttggagggagacagagcttggcggtttgtccattcctctccacgactcaaccctcaaggccccaaagaaactgcccagagaatgattaacaaaagaaggaaagaaacgtggtcacctttctttctttttcttgggggatgaggaagagggtctcgctctgtcatccaggctggagtgcagtggcacaatcacagctcactgcagcctcgaactcctgggttcaagggttcctcccgccccagcctcctagtagctgggactacagacgtgcaccaccacacctggctaatttttcattgtattttaattttttgtagagacggagggctcatcactatgttgccaggctggtctcaaactcctggcctcaagcgatcctcctgcctcagcctcccaaagtgctgggattacaggtgtgagccaccacacctggccacatggttgcccttctgagggatgcaggaatagggtggtgggtggacaaatggatccttctgcaggttcccatctagaacctgccttgaggcagcttttcccccaaggcaaggtcaccgcctcctccatgcacacaggctgggatggggctggtggctacggcagacgcccctcggtccctgagcatgcctggctctgcccccaggtctctgcatcctcgtctcggtggaggtcatgcggcagtcggtgaagcgcatgattgacagtgaggacaccgtctggatcgagtactattactcctggtcctttgcctgcgcctgtgccgccttcatcctcctctttctcggcggtctcgccctcctgctgttctccctgcctcgaatgccccggaacccatgggagtcctgcatggatgctgagcccgagcactaaccctcctgcggccctagcgaccctcaggcttcttccccaggaagcggggtcttggcctggaaccttccagagaggaggcgggagcaattttagccccaccctgctcccatctgcccccctgcaacagtcgcaggctgcttcctctctctgagttcctctgggctgccgcaggctcccctgggaatagagcaagacgtgagtcctaacctggccacagttgggggaggcagagccagcaggtggacaggtgtttgcaggggcccaacttcccctggagctcagaggtgtccccactgtaccagcctctgataagctgcctccagttgtcctttatgaacattgcagggacaacctgtgtttgccagctgggtgttccgtgtaaatagccagcctgtctctttctcggtgataaaacacaccctctctggtgagcccagcgtcccctccttggcttccaggagccctgggaagcatttttaactgggtagaatctgactgtggcttgaaataaaaagctctcagaaaactcatgctttcctggagccttgcatagaaatcgctcggattcccagtcacatcaggggctcccctgacgccgaatgagaaggaaaggccggtcacccgggtatgttgaggtcttttgtggggacaagagggcaacgcacgtggcttagaggctctgaattccaactcgtatctctaggccctgagattgttttcgggagaaactagaaaccctctgatgaagcaagggtgttaaaaataggaggggttatttctagagcctggcatgcaacgcacctggtgccaacacagcgctaaagcagccagccagtgtctgatttctgatgttggatttttcttgtgaagaacatcaggaagcagggcctcgagataacttcgtataatgtatgctatacgaagttatgctaggtaactataacggtcctaaggtagcgatgctcctctacaaacagtgcacatggacacaaacgcagacctgcccagggtcagagg

SEQ ID NO: 29 - DNA, 16551 bp, synthetic construct
  misc_feature 1..16551: note = 6866 knockout allele
  source 1..16551: mol_type = other DNA
ctctggctcagccagacacactaaacttagcttccactcccagctccgtagccactgccaccatctagctagctgcctgggagacactgctgctgccccctgcccagagcacccacacctcaacgaccgccatgggtaccgatttaaatgatccagtggtcctgcagaggagagattgggagaatcccggtgtgacacagctgaacagactagccgcccaccctccctttgcttcttggagaaacagtgaggaagctaggacagacagaccaagccagcaactcagatctttgaacggggagtggagatttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttaactcggcgtttcatctgtggtgcaacgggcgctgggtcggttacggccaggacagtcgtttgccgtctgaatttgacctgagcgcatttttacgcgccggagaaaaccgcctcgcggtgatggtgctgcgctggagtgacggcagttatctggaagatcaggatatgtggcggatgagcggcattttccgtgacgtctcgttgctgcataaaccgactacacaaatcagcgatttccatgttgccactcgctttaatgatgatttcagccgcgctgtactggaggctgaagttcagatgtgcggcgagttgcgtgactacctacgggtaacagtttctttatggcagggtgaaacgcaggtcgccagcggcaccgcgcctttcggcggtgaaattatcgatgagcgtggtggttatgccgatcgcgtcacactacgtctgaacgtcgaaaacccgaaactgtggagcgccgaaatcccgaatctctatcgtgcggtggttgaactgcacaccgccgacggcacgctgattgaagcagaagcctgcgatgtcggtttccgcgaggtgcggattgaaaatggtctgctgctgctgaacggcaagccgttgctgattcgaggcgttaaccgtcacgagcatcatcctctgcatggtcaggtcatggatgagcagacgatggtgcaggatatcctgctgatgaagcagaacaactttaacgccgtgcgctgttcgcattatccgaaccatccgctgtggtacacgctgtgcgaccgctacggcctgtatgtggtggatgaagccaatattgaaacccacggcatggtgccaatgaatcgtctgaccgatgatccgcgctggctaccggcgatgagcgaacgcgtaacgcgaatggtgcagcgcgatcgtaatcacccgagtgtgatcatctggtcgctggggaatgaatcaggccacggcgctaatcacgacgcgctgtatcgctggatcaaatctgtcgatccttcccgcccggtgcagtatgaaggcggcggagccgacaccacggccaccgatattatttgcccgatgtacgcgcgcgtggatgaagaccagcccttcccggctgtgccgaaatggtccatcaaaaaatggctttcgctacctggagagacgcgcccgctgatcctttgcgaatacgcccacgcgatgggtaacagtcttggcggtttcgctaaatactggcaggcgtttcgtcagtatccccgtttacagggcggcttcgtctgggactgggtggatcagtcgctgattaaatatgatgaaaacggcaacccgtggtcggcttacggcggtgattttggcgatacgccgaacgatcgccagttctgtatgaacggtctggtctttgccgaccgcacgccgcatccagcgctgacggaagcaaaacaccagcagcagtttttccagttccgtttatccgggcaaaccatcgaagtgaccagcgaatacctgttccgtcatagcgataacgagctcctgcactggatggtggcgctggatggtaagccgctggcaagcggtgaagtgcctctggatgtcgctccacaaggtaaacagttgattgaactgcctgaactaccgcagccggagagcgccgggcaactctggctcacagtacgcgtagtgcaaccgaacgcgaccgcatggtcagaagccgggcacatcagcgcctggcagcagtggcgtctggcggaaaacctcagtgtgacgctccccgccgcgtcccacgccatcccgcatctgaccaccagcgaaatggatttttgcatcgagctgggtaataagcgttggcaatttaaccgccagtcaggctttctttcacagatgtggattggcgataaaaaacaactgctgacgccgctgcgcgatcagttcacccgtgcaccgctggataacgacattggcgtaagtgaagcgacccgcattgaccctaacgcctgggtcgaacgctggaaggcggcgggccattaccaggccgaagcagcgttgttgcagtgcacggcagatacacttgctgatgcggtgctgattacgaccgctcacgcgtggcagcatcaggggaaaaccttatttatcagccggaaaacctaccggattgatggtagtggtcaaatggcgattaccgttgatgttgaagtggcgagcgatacaccgcatccggcgcggattggcctgaactgccagctggcgcaggtagcagagcgggtaaactggctcggattagggccgcaagaaaactatcccgaccgccttactgccgcctgttttgaccgctgggatctgccattgtcagacatgtataccccgtacgtcttcccgagcgaaaacggtctgcgctgcgggacgcgcgaattgaattatggcccacaccagtggcgcggcgacttccagttcaacatcagccgctacagtcaacagcaactgatggaaaccagccatcgccatctgctgcacgcggaagaaggcacatggctgaatatcgacggtttccatatggggattggtggcgacgactcctggagcccgtcagtatcggcggaattccagctgagcgccggtcgctaccattaccagttggtctggtgtcaaaaataataataaccgggcaggggggatctaagctctagataagtaatgatcataatcagccatatcacatctgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcccccggctagagtttaaacactagaactagtggatccccgggctcgataactataacggtcctaaggtagcgactcgacataacttcgtataatgtatgctatacgaagttatgctaggagggggtaagggttccattttcctctacttctttccatttcttgtcctttcaagactccaagataagttacctgtgggagcagagtttgttggcagatttttctgcaaaggatcaagatggaaacagcctgaggtactttgtatggccagagctaaggtagaaaactagggcatttgtctccagctccatcttgtaaggcctatgttcttcatccagtgcaggtggctggaccagtaagttaagagactccagaaacttctggtcgaccagaccagggacttctggccccaacagcagacactcagaaagactcagtgttgtcttaggtccgttcactccctacacagacaggctagtaacccaaagacctcaggctagaggaaactatggggcagactggctcttagcctctgagacagcagctctcagcctgtgggtcacaacctcttggggctctcatatcagataccctacatatcagatatttacatgattcataacagcagtgagattacagttatgaagtaacagcaaaataagtctatagttgggggccaccataacgcaaggaactgtattaaaggatccttgacattagggaggttgaggccattgctctaggcaaaagtataggcaagattcttcctacctagtttgtcaggctgcgtaacaaaaaaaatcccaggttcctggttcttaagtactgagaatgtcaagttggtgaaggcagagcatggaccaagcatggactccttgggggttggggtgggtcaaagctgccgcatggctataaaacagaaggtagtgtgtagatgctttgttaagcccgtggtcctactatgttttcttccctagtttacatcactcggattctccacgtttccttctgtagtggttgaaaggtattcttccaatccctacccataccttgcaggcaggtccttgcctgggagcttgggtgttagtgtgatacatcatctctattctgccagtatagagcccctgggattcagagcgagcgaagtatgcatggtagaaaagaacatgccagacagaaaagaggcccaggctggagccacagctatccactgccctcttaaaggacctttttttgttttgatacagggtctccaactagttatgcatctagacagtttatgcaattctggggggtggaacccaggcctcagtgggtcacagcatgtgtggcctccatcctgacatagagaacaatgccttctgtccccctgcctaacgcttgtggttcaagcactcagtcctgattcttaaagacccagcagtctgcatgtggagtgcagagcaggccctcccatccttctcctccctcctctttcttagctcttgcttcaggacacccaggcgtcactcatcactgctggggatccagagatttgtaagcagtcttggaaacagagtctcagcatggagggcattgagtggtgtgagcatgtgcttgaaggatatcgtataacctgatgcccacagcccttggccactctacacactcgtaaacagatgcatcatccggccaactccctgggcagggcagaacagggcgaggcagcttctctgggatcgtgcgtgagaagggacattacagttctgtccacctgtcctttgagccatgtgccttttaaagtcagagcagatgaggcatgtctacacagagagagacaggctggcaagaacaccgatctcaggggaccaacaggcaaggcttgcccttgagcctaagcagagcctgtttgtctgccttcttgccctcaccagctgtgaatcacgctgcccacacttgtgacactcaccctatcctcccccagttgtcttccaaagccaacgggcatcaatttccttccacatctaccttttctgtgttccctgggacctctcttttctgtaaaagggctcatacggcgtgctattatttacaaacaattgtagtaacagcatgtccgcatagggctctgtgttatgagggctttgtgtatattaactttgggtaataccaaaaaaaaaaaaaaaaaaaaaaaaagatgagggagagataacccctccaccacccctccaccacccctcctcccactgaatcttctccctcatcgacagcgatgagaattcagggggaaaggaagaagaaacagactctttattttcagaattctggaaattaatcaaaatcttagcgcatcctggcaagtgcttattcaagaaaagcaggcaagttgggagaaagtcaaatttaaggtgctttaatttcctccgtgaaaactactattatctcagtaaaacaagacaaagccaagagagtctggcagacgctagaggaagcagaacaggttgggcctcttgcagagtccctctccaaaacactgccattatgcagattttttttttcctcatcttgcaaaagtcctcaactacaaagctgtgactgggagataagtaggttccatacatgttagaacacctctgtcaactgatggccaagttactagaacagaggtttcatgctcaagaatatagacatctcgaaattagctccaacacagcaacaggaacaagcatcaagaaggagaagcagacttcccgggttgccatgttgtgtgtatgtttaacaaatacaagccattaagaagggcatggcagtatccatctttgatcccagcactggcgaggtaggatatttgtgtctttgaggccagcctggactacatagggaacttcaggacagtcgaggctacatggtgagaccctccttcgaaagcaagcaaacaaaaacaaaacagaaaacaagccatgtgaagacctaagatgatttgggtggtgcatgaagaggagagccagaaactagagtgaatttgaacgagcctttaggatgtctagatgcggacagagactttttttacatttgtttatctgggtgttcgtttgtttctgggggaatgtcagcggatgacgtgagtcagttctctccttccaccgtgtgggttctggaagcccacactcaagtcaggtggtcagcacttgacctgctgagccacctcaccagccctagctaaacggacactttaaatcagccagaaaccgtatctcgagaataaacaagggggcagccgcctcataaaatagataccaatcaagagaaagaaaataatcttaaagcatcaaatagaaatccttttgtaaaaagcaataattaacgtggaaagttcagcagagggattcaaaagcagaactgagcaggcaaatggaaagtctatgacctttaaaaaatgtccactgaggttccgcagggaaaggggcaatgtctcagacatgtgtgagacaccatcatatatatcatgggagtctccagaagaagagaaagaggagagggataatagctaaacgcatctcagactagatgaaaagcatgaacctacaaatgaattctgggtagtgtaaatttaaataaatccatacctccatacatcacatccaaattggttaacgatgcagaaaatgctcaaaacagaaagcatgtacaattctggtgacttatttgaaaacttgatgcaatttagaattgccagaatgcttcaatgaggagttatctacgttggtctctggtgtgcttatgaaagatacattactttgcggctctctgctcttggctttggatgtgacctgctgtttcaaattcctgctccttcaactttcctgctatgaacctgcaattatcacagcaacaggaaatgaaactaagattagcaactgtcctgggttgcttctctgttgctcggataaaacactggctagaaacaacttggggaggaaagggtttctttcggcttactggtcacagtccactctggagggaagccggggcaggaactcaagacagcaacacagaagcaagaactgaacaggggccacagaggagtagaatactgcttgctagcttgctttgcatagcttacccagcttgcttccttatacataggaccacatgcccaggggtggtaccaccccacagtggatgggccctgcaatatccaccattaatcaagaaaacgttcctgaggccagagagatggttcagtggttaagagcatacactgctcttgcagaagacccaagtttggttgccagcattcagctggtggctcacaactgtctgtaactatgctttcagaggatctgatcccctctcctgacctcttctggcactgaatgtacatgctgtgcatatatacactcatgcgcatatacataaacatgaaatgtatattttaaatgaagctgcccccacatacatacgaataggccattctgatggaggcaatttctcaatcgaaattccctttcccggtgaccctaatttgtgtcaagttgacagaaaccaaccagcacagtcacttatcacagacataagatccttgataagataatcagattattttccctgaacaccagggaagcctggcagtaacaggagtccacacatgtgtagctgcttaccacatatcaatatttgggtcaatgatggaccacgtgtaaaagaatcgcctcatggtattatgtcacctagtgatgtcatagttatcccagtttgtgtatgcacacgacataacgtttacacaatgacaacattgcctaacagggcattgctcagaactatccacactgctaagcaatacacaaccataccaactcatccaatctcacagaaacctgcaggagacatatatatatatacacatcatcatcccaggtcacagatgaaggcagtgacgttatcccacgtcacagatgatggacgtgacgctaagaaaatgtgaacaatttaactgaggttatgtagttagagaacaggaatctggtcctaagcatttgtttgtctgctgacttgcttcccatggcttcctcagcttgctttctcgtactacccaggaccacatgcccagggatggtaccacccacagtagtccgagccccgcaacatccaccattaatcaagagctttttcctgaggctggagagatgactcagttactgcatcatacacaccaccaaagaacactgaaggcaagcatggccttgccccgggctaactacctaacaaagagaggctcaatacacgtcttaactcctgatgctccttgttctccagagcttcctataacttccaatgcactatgtccctaaagtgtggtaactcaaggggccacaaggagccccacataggacacgacatggtctgaggcagccagaccaagagcacatacaaggtggtttcactccaccattctcacaggatggaaagcagctgcctcgtgtaaatcttccactgtccaccaagagccatagaaggacctctctgtccaagatcaacacctgagctcagttccaaggtctcctgagtgggaggaggggcttccccagtttataggggagactgtgtgtcagccagtaaaccccacgaggaacattctggctgcccttgtttctttcaggtcctgcctctctagggcaaccccagcatcccatttctctacacccacatctctcctcagtgcagggattgtgggtgtaagaacatgatgtcactgagcctgggaatctctaaggctccctcctctacccacatgaactcatgctgaggcaccctttcctacccacatgatctcatgttgaaggctggagctttaactagaaatcccccccaccccaccccccgccagaagaacaacactatgaggctctgccgaaggccttgggatacccccagcttaggaatcttaggaattcttcagcggtggttaagagagcacacggcatctttataagcttgccaaacacatatacgaacatcctatgtgagactttctccccagctctaacccaatagttcctgaggggttatacatatcaagttgtgttccatatgttaactctgcactacctgctaccctagggaacttctcagtaataactcagagctatcccgtcacctcagcttcaccttggagctccatttgcctgctactctggttcttcgagaatgccacccaggattctgcacagctcgtgactgagccatactgtggggttagtcagccatcccccctgtcccagtctctaggaaagtctgaactgggctcactgtttttgttttgtttccttttgagacaggggttcactatgtaatcctggctggcctgaaactcactctgtagaccaaactggcctggaactcacagagatctgcctgcctctgcctcccatgtgccgggataaaagcgtgcaccaccacatgtggctgtgtataaggagtagccccgttcacatttctacatcactgcgtcttgtataaagaagaaccttgggatcagtataaggcagtttcagtgacaagagagatggctgccagtaagctcgaagactctaattacagtcctggatctgtttggcgcggggacctctgcttacttgctctgaagcactgttggcatcagcacagtgggggaacagagggcaccagggcagactgagaggcccccaaatctatgcaagggtgttatacctagcattggagacaagaaacggcatcggagaggcagaagcaggagggttcaatatctgcaagcaaaagatcaccgcaagcctccccgctccaactttacctgtgacgaagtggccactcagggcctgcttcagatgaggggtgtgtgtgtgtgtgtgtgtgtgtgtgtgtgtgtgtgtgtgtaggtgtgtaggtgtgtaggtgtgtaggtgtgctcacacatatgtgcagatttgcctgaaagacaaacatctacctgaagtgcctcagttattcctcacctcagtgtttgaggcagggtctcccacagagcctggagcccactgatttggttaggctggctggccacatccccagcgacgtgcttgtctctgtcgtaacagcctgagatcacaagtgcaggccaccactatcccacctcccgaaggttctgagaatttgcgggtcctcagacttgcataggaagttctccattgattgagccgtgtgttttagactttccttgatgctcagttgccccaaatactaccatctttcaaaaggataaatatggagaaaactaagtcacatgaaatgtgcccaccagatgagaccctctgccctctgctaaagatgctagcttggttggtctgatgtgactactccatctttgggaccaatgggaactgtgaaagggctcgtggtctctgtcaggcagacacacggactgggaggggatatagttctcacaggttgaaagccaagaatatgggggccttcttcctgtgacaaacccaagagaaaagtaggtcagcctgtctatcctctggaccagagctgagcatactggagagaaactgaggcttccagcattcctaacacaccttaccatcacccacagttgaagatgtttaataaagaaagagacattatatgtgctacccaagggaaagtgtcccccagtaccacctgcctggtccccacaggaatacaacctactccattgcacccaggctcagacatctctgccctgatatccagaatgtgtctgtatattgttgtcagccctgagtgaagcctggcatttatctggtggtggctgtgtccccaacatgctacacaggagtcacctctgcagcccacaagggtcaactatctggggtcaccagagtctcaaggctacctgaaccccacagccctccctctgaaagctcttaactcacagggagtgagtagctgggcgagcggctgaccgtgaaatacacttgagtcggtcaattcttcttggagggtttttcaatggcaactgaaattggcccttaaagaacgagacagaaatgttcctttgtccggagggagaccccggggagacgtgtcacagagcgtgagccgttctgaggcattgattcaagaggatctacagcaacgggttctcataaatataagagggctgcattatgctttaaaatagccgtggatgctgaaagaaaggagcagtgttaagtctgacatcacaaccgctcgccagacaccccaggtaacttggctcaaaagaaaagctatttaaatatagacgcttgggaaaaggcggctggctgccgggaagagtcaccgctgctaggccagatcccatctccaggcaactgtgtcctccaccctaggagaagcggcaattgaactttggaggcgtgggaaggaacaggattaaggagcagtaacatgatacggtcagatagaaggaaccttcatcctgttcaaacacataatggtggcatccccgggcacatatctaaaagaaacagggcgccaccagtccttttggaatgaacattcagctgacactcaatgccattgaaaataagtcttttatactaagggaaacagaaacacaggaaagacaccgaggcagaacctcacataaataaaaagcaacctagtttagttttcttaggaagcagaaaatagggtgcaagatatagttcacttgggagaggttgtacagagcctgaggttccatccccagaccatgtaagccagaagcaatggcaagaacttaaagtcccagtacttgggtggtggaggtaggtgaatcagaattccaagattaccattggctatggttggatatacagtgaattcaaggccatcctgggctacgagggaccctgtctccaaaaaaaaaaaaaacaaaaacaaaaaaacaaagcaaacagggagaaagagagagagagagaattaaaaataacccattcttctagttcttaattcagtaattcatgaataaagaatattgaatgtcttccacaaggattgcaactatattttggtgtcatatctagttgtaaaagagactaagggcagggcagggcagggcaggggtggcacacgcctttaatcccagcacccaggaggcagaggcaggtggttctctgagtttgaggccagtctggtcttcagactgactttcagaacatccagggctacacagagaaaccctatctcaaaaaaggagagagaagagagagagaaagagagggagagaggagagagagagagagagagagagagagagagagagagagagagagagagactaaagaaaggaaaagctggaagtggaattaggcataaggtcatcacggttatatttcctctcagcccccctcctgctgccccgtaacacccaggtctcaaatctaactgtccacaggtcatctatagttgtcatgacaatgctcagtggccaagaagcttgctggggaagattttgtctgaggcccatgtgattcaggtgccgaggggctgaagaagcagcaaacagaagagccctttgtggggcaaagttgggttgccaacctggataagcatcaatcttctagaaactgttctctctctctctctctctctctctctctcatgtgcgtgtgtacacgtgtgacattcatatgggcctgtgtagatatgcatatggagaccaagagctcacatctggtgtcttcccccattgctctctgctttgtttcctgagactgggtctctcactgacctggagctcggcaactggccaatctgctggtccatgagttctaggaatctacctccctgccagtgccaggactacagacacccatcactgaacccagctttttacacagttgcagggaacccccatgctctgtagcaaacacttcaacagcagagccatttccctaggccctggaaatcttttgaaaaatgctgttccctgggtcccactgtgaatggacctggggttcaggtctggtttgatcaaggtgcttcctcatcactgttaatattccgtactggataactgctgtgtcttgtggcttgtgagctgagggcttttggtacaatccttggcctctagacattgcctgggagatggatccatgtgcagagatcactggttaattgctaagcttgcatgccccctactttgggaacaacatgagcccaggtctcagttaccctaactgcctaatgagctcagggtttgcacaccgtccgccctgagattccatcaccctttactccctgaggccatctcagcctccttttggaggctttccttatctgctgaaatgctgtgctaaaagtcagggaactgtttggcccagattcagaatgtgggcggaaacaggtagccagagccagagggcccctctgtgccctgtggcgaaacacctgccctttctaccggctcacagactgtctgttcctttacagaaaagaactgctcctacttcaggcacttcaacccaggggagagctcggaaatctttgaattcaccactcaaaagggtgaggcccttggaagcaggggcggtgggaagcaatttctgtcacaggctgctgagccgccgggagtcacttggcagaaacctgctgactcctgctcaggatgaaaagtagctgcaggctgtgccctgtcacatccctacacacaaggggtggttccccgcccaggttggtgttagggacggtgggtgaagaagacctgccagaaggctgcaagttgcagctggaacgtattccagcagagtccagggcagggagctttgaagggttccaggacctacagtgctggagcaggattctggggtggatgaatggatgggtgagagcctctgtgaggatgtgggagtcctggccacacttaatagatccctgagctgaggaaacatgataatcccatccttcatccatagaactcggagacctcattagcaggtacagcctggcctctgggagaatgagccgccacacagcgtgtaagagctgactcagacacactcatgcacgtgctacagatcctcacgtccgcacatgcacacacagatgggtcttcatgcacaaagcagggaacagacatacttgtatgcatgcatacacatgccaacagaacctcacacatgggacacacatacacactaagagtcttaatgcagggaacactcatgcatgcatgcatgcatacaccagcagaccttcacacatggaaaatgtatacacactgaaagcctaataaagggaacacttatacacacacacaccacgcatacacacacagagatcctcatatatgaggtacatacaagtgtacccacgtatactgacagattctctctctctctctcacacacacatacacacacacacacacacacacacacacacacacacacactcatacaaattctcacatgcagacacagaggcacatactctgatggattcttaggcagcccccacacatccacacccacacactgtcaatcttccggagtccccttgtcaatctgagacagaccacctacagctcagtacctcctctgcctgtcactcaggaggcctttggctctaagaacatctccagccacgtggtgtcccacctcctcctgaggcccttttttcagagaaccctacactttagttccccggggaagatacggatgagttcgtcgacagcacctgtccccactctgccctagaatatagactttgagtaagcctcgctcattcgggtgtaaaatgggaatcctggatgcccagaacttcgaacctcctcgctaagaacactgacagtaggaagtgcctggagaggctctcgctctgaggctccctctcttttgcagagtacagcatctcagcagcggccattgccatcttcagcctcggcttcatcattgtgggttccatctgcgcatttctgtccttcgggaataagcgtgattacctgctgaggccagcatccatgttttatgccttcgcaggtagcgtggggtcctgcagtgtctgagtgggaagaggactcaggttccacacaggaacaaattcagtcagaactcaggtgaccccaggagggctgggagggagaaggcttgctttgttcatttttcccccagtgacacttcccttgggaaggacactggccaaaccttcccagtgagtgattaacaagagaaggaaggaaatgtggcgggtctctgactaacctcctgcaggaggtgggaactggacgtttgggaagtggtaggcgtggatcacttttgtagattcctgcttagaacagtctagaagcagttccctctcaggaccaggtcagaggcagggaacagaggttgggggaggactgaggtggcagcaggagcaccagcagtcactgggactcttggctttgtccccagggctctgcctcatcgtctccgtggaggtcatgaggcagtcggtgaagcgtatgattgacagcgaggacacggtctggatagagcactactattcgtggtctttcgcctgtgcatgtgccgcgttcatcttgctcttcctcggtgggctgttcctcctgctcttctccctgcctcggatgcctcagaacccctgggaatcctgcatggacgctgagccagagcactagatcaaaccagagtctttcggaggtgaggagggagtttcacctctgccctgacctcttgtctctcacgtgtctctccctctgagctcctctacaaacagtgcacatggacacaaacgcagacctgcccagggtcagaggaagcaggccagcaggtatatgaaagggccctcctccgtgcctgccccagctcaggcttgcccaccatgcctgacggctgtgacgatctggtctggtcatctttagaaagcttccgggaacagcctgttctcattctgcagttctgagcatcgcgggtgtcctgagtcagctcctcccccactcttctccgccatgatacgaacccctcctctgtgaactgaacatctctccttggcttccaggattcccaagagacatttttggctggataggatctgtggtttgaaataaaagatctcggaaatgtt

SEQ ID NO: 30 - DNA, 18 bp, Homo sapiens
  source 1..18: mol_type = genomic DNA
ggcgagagctcggagatc

SEQ ID NO: 31 - DNA, 20 bp, Homo sapiens
  source 1..20: mol_type = genomic DNA
ggctgcccaggatgatgaag

SEQ ID NO: 32 - DNA, 27 bp, Homo sapiens
  source 1..27: mol_type = genomic DNA
tcgaattcaccactcagaaggagtaca

SEQ ID NO: 33 - DNA, 22 bp, Mus musculus
  source 1..22: mol_type = genomic DNA
ccgtgcacaacaaagacaagag

SEQ ID NO: 34 - DNA, 20 bp, Mus musculus
  source 1..20: mol_type = genomic DNA
ggctgcccaggatgatgaag

SEQ ID NO: 35 - DNA, 23 bp, Mus musculus
  source 1..23: mol_type = genomic DNA
tgtgagcacgtcacaccatcagg

(19) United States
(12) Patent Application Publication
(10) Pub. No.: US 2025/0017183 A1
(43) Pub. Date: Jan. 16, 2025

(54) NON-HUMAN ANIMALS COMPRISING A MODIFIED CACNG1 LOCUS

(71) Applicant: Regeneron Pharmaceuticals, Inc., Tarrytown, NY (US)

(72) Inventors: Trevor Stitt, Crosswicks, NJ (US); Susannah Brydges, Scarsdale, NY (US); Alexander O. Mujica, Elmsford, NY (US); Roxanne Ally, Briarwood, NY (US)

(21) Appl. No.: 18/707,263

(22) PCT Filed: Nov. 4, 2022
(86) PCT No.: PCT/US2022/079336

§ 371 Date: May 3, 2024

Related U.S. Application Data
(60) Provisional application No. 63/275,582, filed on Nov. 4, 2021.

(51) Int. Cl. A01K 67/0278; A01K 67/0276; C07K 14/47

(52) U.S. Cl. CPC A01K 67/0278; A01K 67/0276; C07K 14/47; A01K 2207/15; A01K 2227/105

(57) ABSTRACT

Non-human animal cells and non-human animals comprising a humanized Cacng1 locus and methods of using such non-human animal cells and non-human animals are provided. Non-human animal cells or non-human animals comprising a humanized Cacng1 locus express a human CACNG1 protein or fragments thereof.

### CROSS-REFERENCE TO RELATED APPLICATION

This application claims benefit of priority to U.S. Provisional Application No. 63/275,582, filed Nov. 4, 2021, which is incorporated by reference in its entirety.

### SEQUENCE LISTING

The Sequence Listing written in file 11102WO01_ST26.txt is 79 kilobytes, was created on Nov. 4, 2022, and is hereby incorproated in its entirety by reference.

### FIELD OF THE INVENTION

A genetically modified non-human animala (e.g., a rodent, e.g., a mouse, or a rat) comprising in its genome a nucleic acid encoding a human Calcium Voltage-Gated Channel Auxiliary Subunit Gamma 1 (CACNG1) protein, or a portion thereof, is described. Thus, genetically modified non-human animals that express human CACNG1 protein, or a portion thereof, e.g., on the surface of a skeletal muscle cell are also described. Such genetically modified non-human animals that express human CACNG1 protein, or a portion thereof, e.g., on the surface of a skeletal muscle cell, may be used as models for preclinical testing of CACNG1-based therapetuics, e.g., CACNG1-based antibodies.

### BACKGROUND

Skeletal muscle is one of the three significant muscle tissues in the human body. Each skeletal muscle contains thousands of muscle fibers wrapped together by connective tissue sheaths. Skeletal muscles allow humans to move and perform daily activities. Skeletal muscles play an essential role in respiratory mechanics and help in maintaining posture and balance. Skeletal muscles also protect the vital organs in the body.

A myriad of medical conditions can occur as a result of abnormalities in the function of skeletal muscles, and a suitable animal model capable of testing therapies directed toward treating aberrant muscle function, e.g., by targeting muscle-specific surface proteins could be helpful for studying medical conditions related to skeletal muscles.

### SUMMARY

Provided herein are genetically modified non-human animals having recombinant genetic loci encoding a human Calcium Voltage-Gated Channel Auxiliary Subunit Gamma 1 (CACNG1) protein. Also provided herein are compositions and methods for generating and using such modified non-human animals.

Described herein are genetically engineered non-human animal genomes, engineered cells, and non-human animals comprising a heterologous (e.g., human) Cacng1 gene, or a portion thereof. In some embodiments, genetically engineered animals described herein express a heterologous (e.g., human) CACNG1 protein from a desired locus (e.g., from an endogenous Cacng1 segment). The non-human animal may be a mammal, such as a rodent (e.g., a mouse or a rat). The non-human animal cell can be a mammalian cell, such as a rodent cell (e.g., a mouse cell or a rat cell). The non-human animal genome can be a mammalian nucleic acid, such as a rodent nucleic acid (e.g., a mouse nucleic acid or a rat nucleic acid).

In some embodiments, a non-human animal, a non-human animal cell, or non-human animal genome comprises a nucleic acid sequence encoding a heterologous (e.g., human) CACNG1 protein or portion thereof.

In some embodiments, the nucleic acid sequence encoding a heterologous (e.g., human) CACNG1 protein or portion thereof comprises: (i) a nucleic acid sequence comprising exon 1 of a human CACNG1 gene or a portion thereo; (ii) a nucleic acid sequence comprising exon 2 of a human CACNG1 gene or a portion thereof, (iii) a nucleic acid sequence comprising exon 3 of a human CACNG1 gene or a portion thereof, (iv) a nucleic acid sequence comprising exon 4 of a human CACNG1 gene or a portion thereof, or (v) any combination of (i)-(iv). In some embodiments, the nucleic acid sequence encoding a heterologous (e.g., human) CACNG1 protein or portion thereof comprises: (i) a nucleic acid sequence comprising exon 1 of a human CACNG1 gene or a portion thereof, (ii) a nucleic acid sequence of intron 1 of a human CACNG1 gene or a portion thereof; (iii) a nucleic acid sequence comprising exon 2 of a human CACNG1 gene or a portion thereof, (iv) a nucleic acid sequence of intron 2 of a human CACNG1 gene or a portion thereof, (v) a nucleic acid sequence comprising exon 3 of a human CACNG1 gene or a portion thereof, (vi) a nucleic acid sequence of intron 3 of a human CACNG1 gene or a portion thereof, (v) a nucleic acid sequence comprising exon 4 of a human CACNG1 gene or a portion thereof, (vii) a nucleic acid sequence of a 3′ untranslated region (UTR) of a human CACNG1 gene; or (v) any combination of (i)-(iv). In some embodiments, the nucleic acid sequence encoding a heterologous CACNG1 protein or portion thereof comprises, consists essentially of, or consists of a nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28.

In some embodiments, the nucleic acid sequence encoding a heterologous (e.g., human) CACNG1 protein or portion thereof is incorporated into endogenous Cacng1 locus (of the genome, cell, or non-human animal). In some embodiments, the nucleic acid sequence encoding a heterologous CACNG1 protein or portion thereof replaces (at an endogenous locus of the non-human animal genome, non-human animal cell, or non-human animal) an orthologous endogenous nucleic acid sequence encoding an endogenous CACNG1 protein or a portion thereof. In some embodiments, the endogenous Cacng1 locus comprises a heterozygous or homozygous replacement of an endogenous nucleic acid sequence encoding an endogenous CACNG1 protein or a portion thereof with the nucleic acid sequence encoding a heterologous CACNG1 protein or portion thereof, wherein the endogenous nucleic acid sequence encoding an endogenous CACNG1 protein or a portion thereof and the nucleic acid sequence encoding a heterologous CACNG1 protein or portion thereof are orthologous.

In some embodiments, the heterologous CACNG1 protein or portion thereof comprises an amino acid sequence of a human CACNG1 protein or portion thereof. In some embodiments, the heterologous CACNG1 protein or portion thereof comprises (i) an amino acid sequence set forth as SEQ ID NO:8; (ii) an amino acid sequence set forth as SEQ ID NO:10; (iii) an amino acid sequence set forth as SEQ ID NO: 12; (iv) an amino acid sequence set forth as SEQ ID NO:14; (v) an amino acid sequence set forth as SEQ ID NO:16; (vi) an amino acid sequence set forth as SEQ ID NO:18; (vii) an amino acid sequence set forth as SEQ ID NO:20; (viii) an amino acid sequence set forth as SEQ ID NO:22; (ix) an amino acid sequence set forth as SEQ ID NO:24; or (x) any combination of (i)-(ii). In some embodiments, the heterologous CACNG1 protein comprises an amino acid sequence set forth as SEQ ID NO: 4.

In some embodiments, a non-human animal cell as decribed herein expresses, on its cell surface, the heterologous CACNG1 protein or portion thereof, which may be a full-length human CACNG1 protein. In some embodiments, a non-human animal cell as described herein is a non-human animal skeletal muscle cell that expresses, on its cell surface, the heterologous (e.g., human) CACNG1 protein or portion thereof.

In some embodiments, a non-human animal cell as described herein is a non-human animal cell that does not express, on its cell surface, the heterologous (e.g., human) CACNG1 protein or portion thereof, e.g., wherein the non-human animal cell is not a skeletal cell and/or, e.g. wherein the non-human animal cell is a pluripotent cell, an embryonic stem cell, a germ cell, etc.

In some embodiments, the non-human animal cell is a mouse cell and the nucleic acid sequence encoding a heterologous CACNG1 protein or portion thereof comprises, consists essentially of, or consists of a nucleic acid sequence set forth as SEQ ID NO: 6.

In some embodiments, a non-human animal as described herein comprises a skeletal muscle cell that expresses, on its cell surface, a heterologous CACNG1 protein or portion thereof. In some embodiments, the non-human animal comprises a non-human skeletal muscle cell that expresses, on its cell surface, a heterologous CACNG1 protein or portion thereof (e.g., a full-length human CACNG1) protein. In some embodiments, the non-human animal comprises a non-human animal cell that comprises a heterologous (e.g., human) Cacng1 gene or a portion thereof and does not express, on its cell surface, the heterologous (e.g., human) CACNG1 protein or portion thereof encoded from the heterologous (e.g., human) Cacng1 gene or portion thereof, e.g., wherein the non-human animal cell not a skeletal cell and/or wherein the non-human animal cell is a pluripotent cell, an embryonic stem cell, a germ cell, etc.

In some embodiments, a non-human animal described herein is a mouse, a non-human animal cell described herein is a mouse cell, or a non-human animal genome described hereni is a mouse nucleic acid, and the nucleic acid sequence encoding a heterologous CACNG1 protein or portion thereof comprises, consists essentially of, or consists of a nucleic acid sequence set forth as SEQ ID NO:6.

Also described herein is a chimeric nucleic acid molecule that encodes a functional CACNG1 protein comprising a nucleic acid sequence of a modified non-human animal Cacng1 gene that encods a non-human CACNG1 protein or portion thereof, wherein the modified non-human animal Cacng1 gene comprises a replacement of a nucleic sequence encoding a portion of the non-human animal CACNG1 protein with a homologous nucleic acid sequence encoding a heterologous CACNG1 protein or portion thereof. In some embodiments, a chimeric nucleic acid molecule as described herein comprises a nucleic acid sequence of a non-human animal Cacng1 gene that (a) encodes a CACNG1 protein and (b) is modified to comprise a replacement of a sequence encoding the CACNG1 protein or portion thereof with a homologous sequence encoding a heterologous CACNG1 protein or a portion thereof, wherein the chimeric nucleic acid molecule encodes a functional CACNG1 protein, and optionally, wherein the chimeric nucleic acid sequence further comprises promoter and/or regulatory sequences of the non-human animal Cacng1 gene. In some embodiments, the homologous nucleic acid sequence comprises: (i) a nucleic acid sequence comprising exon 1 of a human CACNG1 gene or a portion thereof, (ii) a nucleic acid sequence of intron 1 of a human CACNG1 gene or a portion thereof, (iii) a nucleic acid sequence comprising exon 2 of a human CACNG1 gene or a portion thereof, (iv) a nucleic acid sequence of intron 2 of a human CACNG1 gene or a portion thereof, (v) a nucleic acid sequence comprising exon 3 of a human CACNG1 gene or a portion thereof, (vi) a nucleic acid sequence of intron 3 of a human CACNG1 gene or a portion thereof, (v) a nucleic acid sequence comprising exon 4 of a human CACNG1 gene or a portion thereof; (vii) a nucleic acid sequence of a 3′ untranslated region (UTR) of a human CACNG1 gene; or (v) any combination of (i)-(iv). In some embodiments the modified Cacng1 gene further comprises a drug selection cassette. In some embodiments, a chimeric nucleic acid molecule described herein further comprises (i) a 5′ homology arm upstream of the modified non-human animal Cacng1 gene and (ii) a 3′ homology arm downstream of the modified non-human animal Cacng1 gene. In some cases, the 5′ homology arm and 3′ homology arm can undergo homologous recombination with a non-human animal Cacng1 locus of interest, and wherein following homologous recombination with the non-human animal Cacng1 locus of interest, the modified Cacng1 gene can replace the non-human animal Cacng1 gene at the non-human animal Cacng1 locus of interest and is operably linked to an endogenous promoter that drives expression of the non-human animal Cacng1 gene at the non-human animal Cacng1 locus of interest. In specific embodiments, the chimeric nucleic acid described herein has (i) the 5′ homology arm comprising a nucleic acid sequence set forth as SEQ ID NO: 25 and/or; (ii) the 3′ homology arm comprising a nucleic acid sequence set forth as SEQ ID NO:26. In some embodiments, the nucleic acid sequence comprises a nucleic acid sequence set forth as SEQ ID NO:6.

Also described are methods of making a non-human animal, the non-human animal cell, or the non-human animal genome described herein by inserting the nucleic acid sequence encoding the heterologous CACNG1 protein or portion thereof into the genome of the non-human animal, the genome of the non-human animal cell, or the non-human animal genome. In some embodiments, the non-human animal cell is a non-human animal embryonic stem (ES) cell, and wherein the inserting comprises inserting the nucleic acid sequence encoding the heterologous CACNG1 protein or portion thereof into the genome of the non-human animal ES cell to form a modified non-human animal ES cell comprising, in its genome, the nucleic acid sequence encoding the heterologous CACNG1 protein or portion thereof. In some embodiments the method comprises introducing the modified non-human animal ES cell into host embryo cells in vitro. In some embodiments the method comprises gestating, in a suitable non-human surrogate mother animal, the host embryo cells comprising the modified non-human animal ES cell, and allowing the non-human surrogate mother animal to birth non-human animal progeny comprising a germ cell comprising the nucleic acid sequence encoding the heterologous CACNG1 protein or portion thereof. In some embodiments, the nucleic acid sequence encoding the heterologous CACNG1 protein or portion thereof is inserted into an endogenous Cacng1 locus. In such embodiments, the step of inserting comprises replacing an endogenous nucleic sequence encoding an endogenous CACNG1 protein or portion thereof with the nucleic acid sequence encoding the heterologous CACNG1 protein or portion thereof, wherein the endogenous nucleic sequence encoding an endogenous CACNG1 protein or portion thereof and the nucleic acid sequence encoding the heterologous CACNG1 protein or portion thereof are orthologous. In some embodiments, the nucleic acid sequence encoding a heterologous CACNG1 protein or portion thereof comprises: (i) a nucleic acid sequence comprising exon 1 of a human CACNG1 gene or a portion thereof, (ii) a nucleic acid sequence comprising exon 2 of a human CACNG1 gene or a portion thereof, (iii) a nucleic acid sequence comprising exon 3 of a human CACNG1 gene or a portion thereof, (iv) a nucleic acid sequence comprising exon 4 of a human CACNG1 gene or a portion thereof, or (v) any combination of (i)-(iv). In some instnces, the nucleic acid sequence encoding a heterologous CACNG1 protein or portion thereof comprises: (i) a nucleic acid sequence comprising exon 1 of a human CACNG1 gene or a portion thereof, (ii) a nucleic acid sequence of intron 1 of a human CACNG1 gene or a portion thereof, (iii) a nucleic acid sequence comprising exon 2 of a human CACNG1 gene or a portion thereof, (iv) a nucleic acid sequence of intron 2 of a human CACNG1 gene or a portion thereof, (v) a nucleic acid sequence comprising exon 3 of a human CACNG1 gene or a portion thereof, (vi) a nucleic acid sequence of intron 3 of a human CACNG1 gene or a portion thereof, (v) a nucleic acid sequence comprising exon 4 of a human CACNG1 gene or a portion thereof, (vii) a nucleic acid sequence of a 3′ untranslated region (UTR) of a human CACNG1 gene, or (v) any combination of (i)-(iv). In some embodiments, the nucleic acid sequence encoding a heterologous CACNG1 protein or portion thereof comprises, consists essentially of, or consists of a nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28. In some embodiments, the heterologous CACNG1 protein or portion thereof comprises an amino acid sequence of a human CACNG1 protein or portion thereof. In some embodiments, the heterologous CACNG1 protein or portion thereof comprises (i) an amino acid sequence set forth as SEQ ID NO:8 (human cytoplasmic domain 1); (ii) an amino acid sequence set forth as SEQ ID NO:10 (human transmembrane 1); (iii) an amino acid sequence set forth as SEQ ID NO:12 (human extracellular domain 1); (iv) an amino acid sequence set forth as SEQ ID NO:14 (human transmembrane 2); (v) an amino acid sequence set forth as SEQ ID NO:16 (human cytoplasmic domain 2); (vi) an amino acid sequence set forth as SEQ ID NO:18 (human transmembrane 3); (vii) an amino acid sequence set forth as SEQ ID NO:20 (human extracellular domain 3); (viii) an amino acid sequence set forth as SEQ ID NO:22 (human transmembrane 4); (ix) an amino acid sequence set forth as SEQ ID NO:24; (human cytoplasmic domain 4); or (x) any combination of (i)-(ii). In some embodiments, the heterologous CACNG1 protein comprises an amino acid sequence set forth as SEQ ID NO: 4. In some embodiments; (i) the non-human animal is a mammal, such as a rodent; (ii) the non-human animal cell is a mammalian cell, such as a rodent cell; or (iii) the non-human animal genome is a mammalian nucleic acid, such as a rodent nucleic acid. In some embodiments, (i) the non-human animal is rat or a mouse; (ii) the non-human animal cell is a rat cell or a mouse cell; or (iii) the non-human animal genome is a rat nucleic acid or a mouse nucleic acid. In some embodiments, the non-human animal is a mouse, the non-human animal cell is a mouse cell, or the non-human animal genome is a mouse nucleic acid, and the nucleic acid sequence encoding a heterologous CACNG1 protein or portion thereof comprises, consists essentially of, or consists of a nucleic acid sequence set forth as SEQ ID NO: 6.

In some embodiments, the inserting of the nucleic acid comprises contacting the genome of the non-human animal, the genome of the non-human animal cell, or the non-human animal genome with any chimeric nucleic acid molecule of the disclosure.

A non-human animal, non-human animal cell, or non-human animal genome can be made according to any method of the disclosure.

In some embodiments, a non-human animal as described herein comprises an antigen-binding protein that binds a heterologous CACNG1 protein, wherein the non-human animal expresses the heterologous CACNG1 protein or an extracellular domain thereof on a surface of a skeletal muscle cell. In some embodiments, the heterologous CACNG1 protein is a human CACNG1 protein. In some embodiments, the non-human animal is a mouse.

In some embodiments, the non-human animal, non-human animal cell, or non-human animal genome comprises a knockout mutation of an endogenous Cacng1 gene. In some cases, the knockout mutation comprises a deletion of the Cacng1 gene or a portion thereof. In specific embodiments, the knockout mutation comprises a deletion of the entire coding sequence of the Cacng1 gene.

Also provided herein is a non-human animal, non-human animal cell, or non-human animal genome that does not express any CACNG1 protein.

Also provided herein is a non-human animal, non-human animal cell, or non-human animal genome that does not express a protein that is specific to a skeletal muscle, yet the non-human animal, non-human animal cell, or non-human animal genome does not exhibit any gross mutant phenotype.

In some embodiments the disclosure provides a targeting vector comprising: (i) a 5′ homology arm and (ii) a 3′ homology arm, wherein the 5′ homology arm and 3′ homology arm undergo homologous recombination with a non-human animal Cacng1 locus of interest, and wherein following homologous recombination with the non-human animal Cacng1 locus of interest, the targeting vector inserts a knockout mutation in the non-human animal Cacng1 gene at the non-human animal Cacng1 locus of interest.

In some embodiments, the disclosure provides a method of making a CACNG1 knockout non-human animal comprising modifying an endogenous Cacng1 locus of the non-human animal to comprise a knockout mutation.

### BRIEF DESCRIPTION OF THE FIGURES

FIG. 1 is a graph displaying CACNG1 expression (GTEx Portal).

FIG. 2 depicts the strategy (not-to-scale) for creation of a CACNG1 knckout.

FIG. 3A are graphs illustrating that genetic deletion of CACNG1 (CACNG1^{-/-}) in mice does not alter skeletal muscle weight compared to widltype (WT) control animals.

FIG. 3B are graphs illustrating that genetic deletion of CACNG1 (CACNG1^{-/-}) in in mice does not alter twitch (1 Hz) or tetanic (125 Hz) contractile force compared to widltype (WT) control animals.

FIG. 4A shows a schematic (not-to scale) for humanization of the CACNG1^{hu/hu} mice. The asterisks indicate the locations of the upstream (7450hTU) and downstream (7450hTD) primers for the gain-of-allele assay. The top part of the figure illustrates the 12,484 bp sequence derived from the human CACNG1 for humanization of the non-animal genome. The bottom part of the figure illustrates a murine 12,795 bp genomic sequence withon the Cacng1 locus that is targeted for deletion.

FIG. 4B details the strategy (not-to-scale) for CACNG1 humanization of the 7450 allele, including a Neo self-deleting cassette. Replacement of part of coding exon 1, intron 1, coding exons 2-4 (and intervening introns), and 82 bp of 3′ untranslated region (UTR) mouse Cacng1 with the corresponding partial coding exon 1 sequence, intron 1, coding exons 2-4 (and intervening introns), complete 3′ UTR and an additional 158 bp after the 3′ UTR of human CACNG1. 15 bp at the beginning of the coding sequence remains mouse sequence. The loxP-mPrm1-Crei-pA-hUb1-em7-Neo-pA-loxP cassette (4,805 bp) is shown downstream of the human sequence, with the remainder of the mouse 3′ UTR to follow.

FIG. 4C details the strategy (not-to-scale) for CACNG1 humanization of the 7451 allele, where a cassette is deleted, and a LoxP site remains. Replacement of part of coding exon 1, intron 1, coding exons 2-4 (and intervening introns), and 82 bp of 3′ untranslated region (UTR) mouse Cacng1 with the corresponding partial coding exon 1 sequence, intron 1, coding exons 2-4 (and intervening introns), complete 3′ UTR and an additional 158 bp after the 3′ UTR of human CACNG1. 15 bp at the beginning of the coding sequence remains mouse sequence. The loxP-mPrm1-Crei-pA-hUb1-em7-Neo-pA-loxP cassette (4,805 bp) is shown downstream of the human sequence, with the remainder of the mouse 3′ UTR to follow. After cassette deletion, LoxP and cloning sites (77 bp) remain following human 3′ UTR.

FIG. 5 shows an alignment of the mouse CACNG1 protein (mCACNG1; SEQ ID NO:2) with the human hCACNG1 protein (hCACNG1; SEQ ID NO:4), and the CACNG1 protein encoded by the 7451 allele (7451; SEQ ID NO:4). The asterisks denote residues that remain unchanged. The heavy solid line denotes the transmembrane domains. The underscored residues are those encoded by the introduced human exons. The cytoplasmic and the extracellular domains are labeled and shown.

FIG. 6A are graphs demonstrating that the expression of mouse CACNG1 (mCACNG1) is not detectable by qPCR in CACNG1^{hu/hu} mouse muscle (left graph), while human CACNG1 (hCACNG1) is expressed in CACNG1^{hu/hu}, but not WT mouse muscle (right graph).

FIG. 6B are images illustrating live staining of single skeletal myofibers with 100 nM of Alexa 647-conjugated human-specific α-CACNG1 Ab showing binding to myofibers isolated from CACNG1^{hu/hu} mice, but not to myofibers isolated from WT mice.

FIG. 6C are images illustrating cryo-fluorescence tomography (CryoFT) images of CACNG1^{hu/hu} mice injected with 10 mg/kg Alexa 647-conjugated human-specific α-CACNG1 Ab showing high specificity for skeletal muscle compared to isotype control Ab 6 days following injection.

FIG. 7 are images illustrating histological sections from CACNG1^{hu/hu} mice dosed with 10 mg/kg Alexa 647-conjugated human-specific α-CACNG1 Ab shows binding to skeletal muscle 6 days following injection. Top panel displays endogenous Alexa 647 signal from Abs that were injected in vivo and bottom panel displays an overlay of Alexa647-Ab binding with laminin and DAPI co-staining to visualize muscle morphology.

FIG. 8A provides an annotation of the cytoplasmic domains (amino acids 1-10, 131-135, and 206-223), the transmembrane domains (amino acids 11-29, 110-130, 136-156, and 181-205), and the extracellular domains (amino acids 30-109 and 157-180) of the mouse Cacng1 protein referenced by NP_031608.1.

FIG. 8B provides an annotation of the nucleic acid sequences encoding the cytoplasmic domains (nucleic acids 1-30, 391-405, and 616-669), the transmembrane domains (nucleic acids 31-87, 328-390, 406-468, and 541-615), and the extracellular domains (nucleic acids 88-327 and 469-540) of the mouse coding DNA sequence (CDS).

FIG. 9A provides an annotation of the cytoplasmic domains (amino acids 1-10, 130-134, and 205-222), the transmembrane domains (amino acids 11-29, 109-129, 135-155, and 180-204), and the extracellular domains (amino acids 30-108 and 156-179) of the human CACNG1 protein referenced by NP_000718.1.

FIG. 9B provides an annotation of the nucleic acid sequences encoding the cytoplasmic domains, the transmembrane domains, and the extracellular domains, of the human coding DNA sequence (CDS).

FIG. 10A provides an illustrative sequence for the CACNG1 protein encoded by the 7451 allele.

FIG. 10B provides an illustrative sequence for the mouse/human CACNG1 nucleic acid coding sequence (CDS), including the 3′ untranslated sequence.

FIG. 11 provides a nucleic acid sequence for the 7450 Allele. CACNG1 humanized region with Neo self deleting cassette=mouse (lowercase)_HUMAN_XhoI_LoxP_Prm_Crei_sv40 polya (lowercase)-hUbi-em7 (lowercase)-NEO-PGK polyA_LoxP_ICeUI_mouse (lowercase).

FIG. 12 provides a nucleic acid sequence for the 7450 Allele. CACNG1 humanized region with Neo self deleting cassette=mouse (lowercase)_HUMAN_XhoI_LoxP_Prm_Crei_sv40 polya (lowercase)-hUbi-em7 (lowercase)-NEO-PGK polvA_LoxP_ICeUI_mouse (lowercase).

### DEFINITIONS

The terms “protein,” “polypeptide,” and “peptide,” are used interchangeably herein, and include polymeric forms of amino acids of any length, including coded and non-coded amino acids and chemically or biochemically modified or derivatized amino acids. The terms also include polymers that have been modified, such as polypeptides having modified peptide backbones. The term domain can refer to any part of a protein or polypeptide having a particular function or structure.

Proteins are said to have an “N-terminus” and a “C-terminus.” The term “N-terminus” relates to the start of a protein or polypeptide, terminated by an amino acid with a free amine group (—NH₂). The term “C-terminus” relates to the end of an amino acid chain (protein or polypeptide), terminated by a free carboxyl group (—COOH).

The terms “nucleic acid” and “polynucleotide,” used interchangeably herein, include polymeric forms of nucleotides of any length, including ribonucleotides, deoxyribonucleotides, or analogs or modified versions thereof. Nucleic acids and polynucleotides can include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “genomically integrated” refers to a nucleic acid that has been introduced into a cell such that the nucleotide sequence integrates into the genome of the cell and is capable of being inherited by progeny thereof. Any protocol may be used for the stable incorporation of a nucleic acid into the genome of a cell.

The term “targeting vector” refers to a recombinant nucleic acid that can be introduced by homologous recombination, non-homologous-end-joining-mediated ligation, or any other means of recombination to a target position in the genome of a cell.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “wild type” includes entities having a structure and/or activity as found in a normal (as contrasted with mutant, diseased, altered, or so forth) state or context. Wild type genes and polypeptides often exist in multiple different forms (e.g., alleles).

The expression “gross mutant phenotype” refers to a significant difference or variation in phenotype between an engineered non-human mouse of the disclosure and a “wild type.”

The term “endogenous” refers to a nucleic acid sequence that occurs naturally within a cell or non-human animal. For example, an endogenous Cacng1 sequence of a non-human animal refers to a native Cacng1 sequence that naturally occurs at the Cacng1 locus in the non-human animal.

“Exogenous” molecules or sequences include molecules or sequences that are not normally present in a cell in that form. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two portions that do not naturally occur together in the same molecule. For example, the term “heterologous,” when used with reference to portions of a nucleic acid or portions of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by sequences not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

The term “locus” refers to a specific location of a gene (or significant sequence), DNA sequence, polypeptide-encoding sequence, or position on a chromosome of the genome of an organism. For example, an “Cacng1 locus” may refer to the specific location of an Cacng1 gene, Cacng1 DNA sequence, Cacng1-encoding sequence, or Cacng1 position on a chromosome of the genome of an organism that has been identified as to where such a sequence resides. An “Cacng1 locus” may comprise a regulatory element of an Cacng1 gene, including, for example, an enhancer, a promoter, 5′ and/or 3′ untranslated region (UTR), or a combination thereof.

The term “gene” refers to a DNA sequence in a chromosome that codes for a product (e.g., an RNA product and/or a polypeptide product) and includes the coding region interrupted with non-coding introns and sequence located adjacent to the coding region on both the 5′ and 3′ ends such that the gene corresponds to the full-length mRNA (including the 5′ and 3′ untranslated sequences). The term “gene” also includes other non-coding sequences including regulatory sequences (e.g., promoters, enhancers, and transcription factor binding sites), polyadenylation signals, internal ribosome entry sites, silencers, insulating sequence, and matrix attachment regions. These sequences may be close to the coding region of the gene (e.g., within 10 kb) or at distant sites, and they influence the level or rate of transcription and translation of the gene.

The term “allele” refers to a variant form of a gene. Some genes have a variety of different forms, which are located at the same position, or genetic locus, on a chromosome. A diploid organism has two alleles at each genetic locus. Each pair of alleles represents the genotype of a specific genetic locus. Genotypes are described as homozygous if there are two identical alleles at a particular locus and as heterozygous if the two alleles differ.

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “variant” refers to a nucleotide sequence differing from the sequence most prevalent in a population (e.g., by one nucleotide) or a protein sequence different from the sequence most prevalent in a population (e.g., by one amino acid).

The term “fragment” when referring to a protein means a protein that is shorter or has fewer amino acids than the full-length protein. The term “fragment” when referring to a nucleic acid means a nucleic acid that is shorter or has fewer nucleotides than the full-length nucleic acid. A fragment can be, for example, an N-terminal fragment (i.e., removal of a portion of the C-terminal end of the protein), a C-terminal fragment (i.e., removal of a portion of the N-terminal end of the protein), or an internal fragment.

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

A “homologous” sequence (e.g., nucleic acid sequence) includes a sequence that is either identical or substantially similar to a known reference sequence, such that it is, for example, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to the known reference sequence. Homologous sequences can include, for example, orthologous sequence and paralogous sequences. Homologous genes, for example, typically descend from a common ancestral DNA sequence, either through a speciation event (orthologous genes) or a genetic duplication event (paralogous genes). “Orthologous” genes include genes in different species that evolved from a common ancestral gene by speciation. Orthologs typically retain the same function in the course of evolution. “Paralogous” genes include genes related by duplication within a genome. Paralogs can evolve new functions in the course of evolution.

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

The term “reporter gene” refers to a nucleic acid having a sequence encoding a gene product (typically an enzyme) that is easily and quantifiably assayed when a construct comprising the reporter gene sequence operably linked to a heterologous promoter and/or enhancer element is introduced into cells containing (or which can be made to contain) the factors necessary for the activation of the promoter and/or enhancer elements. Examples of reporter genes include, but are not limited, to genes encoding beta-galactosidase (lacZ), the bacterial chloramphenicol acetyltransferase (cat) genes, firefly luciferase genes, genes encoding beta-glucuronidase (GUS), and genes encoding fluorescent proteins. A “reporter protein” refers to a protein encoded by a reporter gene.

The term “fluorescent reporter protein” as used herein means a reporter protein that is detectable based on fluorescence wherein the fluorescence may be either from the reporter protein directly, activity of the reporter protein on a fluorogenic substrate, or a protein with affinity for binding to a fluorescent tagged compound. Examples of fluorescent proteins include green fluorescent proteins (e.g., GFP, GFP-2, tagGFP, turboGFP, eGFP, Emerald, Azami Green, Monomeric Azami Green, CopGFP, AceGFP, and ZsGreen1), yellow fluorescent proteins (e.g., YFP, eYFP, Citrine, Venus, YPet, PhiYFP, and ZsYellow1), blue fluorescent proteins (e.g., BFP, eBFP, eBFP2, Azurite, mKalama1, GFPuv, Sapphire, and T-sapphire), cyan fluorescent proteins (e.g., CFP, eCFP, Cerulean, CyPet, AmCyan1, and Midoriishi-Cyan), red fluorescent proteins (e.g., RFP, mKate, mKate2, mPlum, DsRed monomer, mCherry, mRFP1, DsRed-Express, DsRed2, DsRed-Monomer, HcRed-Tandem, HcRed1, AsRed2, eqFP611, mRaspberry, mStrawberry, and Jred), orange fluorescent proteins (e.g., mOrange, mKO, Kusabira-Orange, Monomeric Kusabira-Orange, mTangerine, and tdTomato), and any other suitable fluorescent protein whose presence in cells can be detected by flow cytometry methods.

The term “recombination” includes any process of exchange of genetic information between two polynucleotides and can occur by any mechanism. Recombination in response to double-strand breaks (DSBs) occurs principally through two conserved DNA repair pathways: non-homologous end joining (NHEJ) and homologous recombination (HR). See Kasparek & Humphrey (2011) Seminars in Cell & Dev. Biol. 22:886-897, herein incorporated by reference in its entirety for all purposes. Likewise, repair of a target nucleic acid mediated by an exogenous donor nucleic acid can include any process of exchange of genetic information between the two polynucleotides.

NHEJ includes the repair of double-strand breaks in a nucleic acid by direct ligation of the break ends to one another or to an exogenous sequence without the need for a homologous template. Ligation of non-contiguous sequences by NHEJ can often result in deletions, insertions, or translocations near the site of the double-strand break. For example, NHEJ can also result in the targeted integration of an exogenous donor nucleic acid through direct ligation of the break ends with the ends of the exogenous donor nucleic acid (i.e., NHEJ-based capture). Such NHEJ-mediated targeted integration can be preferred for insertion of an exogenous donor nucleic acid when homology directed repair (HDR) pathways are not readily usable (e.g., in non-dividing cells, primary cells, and cells which perform homology-based DNA repair poorly). In addition, in contrast to homology-directed repair, knowledge concerning large regions of sequence identity flanking the cleavage site is not needed, which can be beneficial when attempting targeted insertion into organisms that have genomes for which there is limited knowledge of the genomic sequence. The integration can proceed via ligation of blunt ends between the exogenous donor nucleic acid and the cleaved genomic sequence, or via ligation of sticky ends (i.e., having 5′ or 3′ overhangs) using an exogenous donor nucleic acid that is flanked by overhangs that are compatible with those generated by a nuclease agent in the cleaved genomic sequence. See, e.g., US 2011/020722, WO 2014/033644, WO 2014/089290, and Maresca et al. (2013) Genome Res. 23(3):539-546, each of which is herein incorporated by reference in its entirety for all purposes. If blunt ends are ligated, target and/or donor resection may be needed to generation regions of microhomology needed for fragment joining, which may create unwanted alterations in the target sequence.

Recombination can also occur via homology directed repair (HDR) or homologous recombination (HR). HDR or HR includes a form of nucleic acid repair that can require nucleotide sequence homology, uses a “donor” molecule as a template for repair of a “target” molecule (i.e., the one that experienced the double-strand break), and leads to transfer of genetic information from the donor to target. Without wishing to be bound by any particular theory, such transfer can involve mismatch correction of heteroduplex DNA that forms between the broken target and the donor, and/or synthesis-dependent strand annealing, in which the donor is used to resynthesize genetic information that will become part of the target, and/or related processes. In some cases, the donor polynucleotide, a portion of the donor polynucleotide, a copy of the donor polynucleotide, or a portion of a copy of the donor polynucleotide integrates into the target DNA. See Wang et al. (2013) Cell 153:910-918; Mandalos et al. (2012) PLOS ONE 7:e45768:1-9; and Wang et al. (2013) Nat Biotechnol. 31:530-532, each of which is herein incorporated by reference in its entirety for all purposes.

The term “antigen-binding protein” includes any protein that binds to an antigen. Examples of antigen-binding proteins include an antibody, an antigen-binding fragment of an antibody, a multispecific antibody (e.g., a bi-specific antibody), an scFV, a bis-scFV, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

The term “multi-specific” or “bi-specific” with reference to an antigen-binding protein means that the protein recognizes different epitopes, either on the same antigen or on different antigens. A multi-specific antigen-binding protein can be a single multifunctional polypeptide, or it can be a multimeric complex of two or more polypeptides that are covalently or non-covalently associated with one another. For example, an antibody or fragment thereof can be functionally linked (e.g., by chemical coupling, genetic fusion, non-covalent association or otherwise) to one or more other molecular entities, such as a protein or fragment thereof to produce a bispecific or a multi-specific antigen-binding molecule with a second binding specificity.

The term “antigen” refers to a substance, whether an entire molecule or a domain within a molecule, which is capable of eliciting production of antibodies with binding specificity to that substance. The term antigen also includes substances, which in wild type host organisms would not elicit antibody production by virtue of self-recognition, but can elicit such a response in a host animal with appropriate genetic engineering to break immunological tolerance.

The term “epitope” refers to a site on an antigen to which an antigen-binding protein (e.g., antibody) binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996), herein incorporated by reference in its entirety for all purposes.

An antibody paratope as described herein generally comprises at a minimum a complementarity determining region (CDR) that specifically recognizes the heterologous epitope (e.g., a CDR3 region of a heavy and/or light chain variable domain).

The term “antibody” includes immunoglobulin molecules comprising four polypeptide chains, two heavy (H) chains and two light (L) chains inter-connected by disulfide bonds. Each heavy chain comprises a heavy chain variable domain and a heavy chain constant region (C_{H}). The heavy chain constant region comprises three domains: C_{H}1, C_{H}2 and C_{H}3. Each light chain comprises a light chain variable domain and a light chain constant region (C_{L}). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3). The term “high affinity” antibody refers to an antibody that has a K_{D} with respect to its target epitope about of 10⁻⁹ M or lower (e.g., about 1×10⁻⁹ M, 1×10⁻¹⁰ M, 1×10⁻¹¹ M, or about 1×10⁻¹² M). In one embodiment, K_{D} is measured by surface plasmon resonance, e.g., BIACORE™; in another embodiment, K_{D} is measured by ELISA.

The term “bispecific antibody” includes an antibody capable of selectively binding two or more epitopes. Bispecific antibodies generally comprise two different heavy chains, with each heavy chain specifically binding a different epitope-either on two different molecules (e.g., on two different antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding or one or both of the heavy chains to one or both epitopes.

The term “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an epitope (e.g., recognizing the epitope with a K_{D} in the micromolar, nanomolar, or picomolar range), that is capable of expressing and secreting from a cell, and that comprises at least one CDR. Heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the World Wide Web (www) at the URL “imgt.org.”

The term “light chain” includes an immunoglobulin light chain sequence from any organism, and unless otherwise specified includes human kappa (κ) and lambda (λ) light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region amino acid sequence. Light chain variable domains are encoded by the light chain variable region nucleotide sequence, which generally comprises light chain V_{L} and light chain J_{L} gene segments, derived from a repertoire of light chain V and J gene segments present in the germline. Sequences, locations and nomenclature for light chain V and J gene segments for various organisms can be found in IMGT database, which is accessible via the internet on the World Wide Web (www) at the URL “imgt.org.” Light chains include those, e.g., that do not selectively bind either a first or a second epitope selectively bound by the epitope-binding protein in which they appear. Light chains also include those that bind and recognize, or assist the heavy chain with binding and recognizing, one or more epitopes selectively bound by the epitope-binding protein in which they appear.

The term “complementary determining region” or “CDR,” as used herein, includes an amino acid sequence encoded by a nucleic acid sequence of an organism's immunoglobulin genes that normally (i.e., in a wild type animal) appears between two framework regions in a variable region of a light or a heavy chain of an immunoglobulin molecule (e.g., an antibody or a T cell receptor). A CDR can be encoded by, for example, a germline sequence or a rearranged sequence, and, for example, by a naïve or a mature B cell or a T cell. A CDR can be somatically mutated (e.g., vary from a sequence encoded in an animal's germline), humanized, and/or modified with amino acid substitutions, additions, or deletions. In some circumstances (e.g., for a CDR3), CDRs can be encoded by two or more sequences (e.g., germline sequences) that are not contiguous (e.g., in an unrearranged nucleic acid sequence) but are contiguous in a B cell nucleic acid sequence, e.g., as a result of splicing or connecting the sequences (e.g., V-D-J recombination to form a heavy chain CDR3.

Specific binding of an antigen-binding protein to its target antigen includes binding with an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas non-specific binding is usually the result of van der Waals forces. Specific binding does not however necessarily imply that an antigen-binding protein binds one and only one target.

“Optional” or “optionally” means that the subsequently described event or circumstance may or may not occur and that the description includes instances in which the event or circumstance occurs and instances in which it does not.

Designation of a range of values includes all integers within or defining the range, and all subranges defined by integers within the range.

Unless otherwise apparent from the context, the term “about” encompasses values within a standard margin of error of measurement (e.g., SEM) of a stated value.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

### DETAILED DESCRIPTION

### I. Overview

Disclosed herein are non-human animal cells, non-human animals, and non-human genomes comprising an exogenous sequence found to be specifically expressed in skeletal muscle, and reagents for making the same. In some embodiments, the exogenous sequence is incorporated in the endogenous locus of a gene.

Skeletal muscle is one of the three significant muscle tissues in the human body. Each skeletal muscle consists of thousands of muscle fibers wrapped together by connective tissue sheaths. The individual bundles of muscle fibers in a skeletal muscle are known as fasciculi. The outermost connective tissue sheath surrounding the entire muscle is known as epimysium. The connective tissue sheath covering each fasciculus is known as perimysium, and the innermost sheath surrounding individual muscle fiber is known as endomysium. Each muscle fiber contains myofibrils containing multiple myofilaments.

When bundled together, all the myofibrils get arranged in a unique striated pattern forming sarcomeres, which are the fundamental contractile unit of a skeletal muscle. The two most significant myofilaments are actin and myosin filaments arranged distinctively to form various bands on the skeletal muscle. The stem cells that differentiate into mature muscle fibers are known as satellite cells that can be found between the basement membrane and the sarcolemma (the cell membrane surrounding the striated muscle fiber cell). When stimulated by growth factors, the stem cells differentiate and multiply to form new muscle fiber cells.

The primary functions of the skeletal muscle take place via the intrinsic excitation-contraction coupling process of the skeletal muscle. As the muscle is attached to the bone tendons, the contraction of the muscle leads to movement of that bone that allows for the performance of specific movements. The skeletal muscle also provides structural support and helps in maintaining the posture of the body. The skeletal muscle also acts as a storage source for amino acids that can be used by different organs of the body for synthesizing organ-specific proteins. The skeletal muscle also plays a central role in maintaining thermostasis and acts as an energy source during starvation. The CACNG1 protein has been found to be specifically expressed in skeletal muscle.

In some embodiments, provided herein are non-human animal cells and non-human animals having a heterologous Cacng1 sequence in the genomes of the non-human animal cells or non-human animals provided herein. The heterologous Cacng1 sequence can be inserted into an endogenous Cacng1 locus, thus providing non-human animal cells and non-human animals having a genetically modified endogenous Cacng1 locus.

In some embodiments, provided herein are nucleic acids encoding heterologous sequences encoding at least a portion of a Cacng1 sequence, and methods for making non-human animal cells and non-human animals with such nucleic acids. In some embodiments, such nucleic acids have sequences to facilitate the editing of the non-human animal (e.g., loxP sites) flanking the sequences encoding the Cacng1 gene.

In some embodiments, provided herein are antibodies against a chimeric CACNG1 protein(s) produced by a non-human animal cell and/or a non-human animal of the disclosure.

In some embodiments, the disclosure provides methods that can be used for making such non-human animals (e.g., a rodent, e.g., a rat or a mouse), cells and/tissues derived from such non-human animals, and nucleotides (e.g., targeting vectors, genomes, etc.).

In some embodiments, the disclosure also provides a non-human animal genome comprising a genetically modified endogenous CACNG1 locus having a heterologous Cacng1 sequence. In some embodiments, the heterologous Cacng1 sequence encodes a CACNG1 human protein sequence. In some cases, all or part of a CACNG1 domain is encoded by a segment of an endogenous Cacng1 locus that has been deleted and replaced with a heterologous Cacng1 sequence.

In some embodiments, non-human animals comprising a humanized Cacng1 locus and expressing a humanized or chimeric CACNG1 protein from the humanized Cacng1 locus are provided, as well as methods of using such non-human animals (e.g., a rodent, e.g., a rat or a mouse), cells and/tissues derived from such non-human animals, and nucleotides (e.g., targeting vectors, genomes, etc.) useful for making such animals.

In some embodiments, described herein are non-human animals comprising a genetically modified Cacng1 locus encoding a modified CACNG1 protein, wherein the modified CACNG1 protein comprises a domain of a human CACNG1 sequence, and all or part of the domain is encoded by a segment of the endogenous Cacng1 locus that has been deleted and replaced with an orthologous human CACNG1 sequence, and wherein the non-human animal expresses the modified Cacng1 protein.

In some embodiments, a domain of the human CACNG1 sequence is encoded by the segment of the endogenous Cacng1 locus that has been deleted and replaced with a heterologous sequence. Such domains can be a human Cacng1 extracellular domain. Suitable sequences encoding extracellular domains contemplated by the disclosure include the human extracellular domains corresponding to amino acids 30-108 (SEQ ID NO: 12), amino acids 156-179 (SEQ ID NO:20), or both, of the CACNG1 protein upon translation within a cell.

In some embodiments, at least two domains of the human CACNG1 sequence are encoded by a segment of the endogenous Cacng1 locus in a humanized mouse model. Illustrative examples of non-limiting domains of the human CACNG1 sequence contain a cytoplasmic domain, a transmembrane domain, and an extracellular domain. In some cases, all or part of each domain can be encoded by the segment of the endogenous Cacng1 locus that has been deleted and replaced with an orthologous human CACNG1 sequence. In other cases, the cytoplasmic domain and the extracellular domain can be optionally encoded by endogenous genome. In some embodiments, all or part of both the cytoplasmic domain and the transmembrane domain are encoded by the segment of the endogenous Cacng1 locus that has been deleted and replaced with an orthologous human CACNG1 sequence. In some embodiments, all the cytoplasmic domain, the transmembrane domain, and the extracellular domain are encoded by the segment of the endogenous Cacng1 locus that has been deleted and replaced with an orthologous human CACNG1 sequence. The latter incorporates multiple humanized domains of the human CACNG1 gene into a non-human genome; the former allows for humanization of the extracellular membrane, while preserving endogenous domains of the domains that are understood to be located within a membrane and within a cell. Suitable sequences encoding the cytoplasmic domain(s) of the disclosure produce the human cytoplasmic domains corresponding to amino acids 1-10 (SEQ ID NO:8), amino acids 130-134 (SEQ ID NO:16), amino acids 205-222 (SEQ ID NO:24), or any combination thereof, of the CACNG1 protein upon translation within a cell. Suitable sequences encoding the transmembrane domain(s) of the disclosure produce the human transmembrane domain(s) corresponding to amino acids 11-29 (SEQ ID NO:10), amino acids 109-129 (SEQ ID NO:14), amino acids 135-155 (SEQ ID NO:18), amino acids 180-204 (SEQ ID NO:22), or any combination thereof, of the CACNG1 protein upon translation within a cell. Consequently, in some alternative embodiments all or part of a cytoplasmic domain or the transmembrane domain is encoded by an endogenous non-human animal Cacng1 sequence.

In some embodiments, the non-human animal or non-human animal genome described herein encodes an orthologous human CACNG1 sequence in place of an endogenous mouse Cacng1 sequence. In some embodiments, the non-human animal or non-human animal genome comprises the sequence selected from the group consising of a nucleic acid sequence set forth as SEQ ID NO:5, a nucleic acid sequence set forth as SEQ ID NO:27, and a nucleic acid sequence set forth as SEQ ID NO:28.

In some embodiments, the human CACNG1 sequence that is encoded by the segment of the endogenous Cacng1 locus that has been deleted and replaced with a human Cacng1 sequence encoding a full-length 71 domain of a voltage-dependent calcium channel.

In some embodiments the non-human animal or non-human animal genome described herein is heterozygous for the genetically modified endogenous Cacng1 locus. In some embodiments, the non-human animal or non-human animal genome is homozygous for the genetically modified endogenous Cacng1 locus.

In some embodiments, segments of an endogenous Cacng1 locus are deleted and replaced with an exogenous Cacng1 sequence. In some of these cases, the endogenous Cacng1 locus that has been deleted can comprise a segment of the 3′ untranslated region, a segment of coding exon 1, a segment of intron 1, a segment of coding exon 2, a segment of intron 2, a segment of coding exon 3, a segment of intron 3, a segment of coding exon 4, or a combination of the aforementioned segments of the endogenous Cacng1 locus.

In some embodiments, a human CACNG1 sequence may be used to replace a locus within a non-human animal or non-human cell. In such embodiments the orthologous human CACNG1 sequence that replaces the segment of the endogenous locus may comprise a segment of anyone of the 3′ untranslated region of the human CACNG1 sequence, exon 1 of the human CACNG1 sequence, intron 1 of the human CACNG1 sequence, exon 2 of the human CACNG1 sequence, intron 2 of the human CACNG1 sequence, exon 2 of the human CACNG1 sequence, intron 3 of the human CACNG1 sequence, exon 3 of the human CACNG1 sequence, intron 4 of the human CACNG1 sequence, exon 4 of the human CACNG1 sequence, or any combination thereof.

In some embodiments, the non-human animal is a mammal, or the non-human animal genome is a mammalian genome. In some embodiments, the non-human animal can be a rodent, or the non-human animal genome can be a rodent genome. In some embodiments, the non-human animal can be a rat or mouse, or the non-human animal genome can be a rat genome or a mouse genome.

In some embodiments, the heterologous sequence incorporated on the genome of the non-human animal or the non-human animal genome encodes a human Cacng1 extracellular domain, a human Cacng1 transmembrane domain, and a human Cacng1 domain.

In some embodiments, the heterologous sequence incorporated on the genome of the non-human animal or the non-human animal genome encodes at least two domains of the human CACNG1 sequence. Non-limiting examples of two or more domains include a first cytoplasmic domain, a first transmembrane domain, a first extracellular domain, a second transmembrane domain, a second cytoplasmic domain, a third transmembrane domain, a second extracellular domain, a fourth transmembrane domain, a third cytoplasmic domain.

In some embodiments, a heterologous sequence incorporated on the genome of the non-human animal or the non-human animal genome comprises a suitable sequence for encoding amino acids 1-10 (cytoplasmic domain), amino acids 11-29 (transmembrane domain), amino acids 30-108 (extracellular domain), amino acids 109-129 (transmembrane domain), amino acids 130-134 (cytoplasmic domain), amino acids 135-155 (transmembrane domain), amino acids 156-179 (extracellular domain), amino acids 180-204 (transmembrane domain), amino acids 205-222 cytoplasmic domain or any suitable combination thereof.

In some embodiments, provided herein is a non-human animal cell comprising a genetically modified endogenous Cacng1 locus encoding a modified CACNG1 protein, wherein the modified Cacng1 protein comprises a domain of a human CACNG1 sequence, and all or part of the domain is encoded by a segment of the endogenous Cacng1 locus that has been deleted and replaced with an orthologous human CACNG1 sequence. The non-human animal cell can be a skeletal muscle cell, a pluripotent cell, an ES cell, or a germ cell.

In some embodiments, the disclosure further provides methods for making any non-human animal, or reagents required for making the non-human animal as described herein.

### A. Calcium Voltage-Gated Channel Auxiliary Subunit Gamma 1 (CACNG1)

The cells and non-human animals described herein generally contain an exogenous sequence encoding a segment of a CACNG1 protein (e.g., a human CACNG1 protein domain). Voltage-dependent calcium channels are generally composed of five subunits. The protein encoded by the CACNG1 gene represents one of these subunits. Further, the protein encoded by the CACNG1 gene, gamma, is one of two known gamma subunit proteins. This particular gamma subunit is part of skeletal muscle 1,4-dihydropyridine-sensitive calcium channels and is an integral membrane protein that plays a role in excitation-contraction coupling. This gene is part of a functionally diverse eight-member protein subfamily of the PMP-22/EMP/MP20 family and is located in a cluster with two family members that function as transmembrane AMPA receptor regulatory proteins (TARPs).

The gene encoding human CACNG1 (CACNG1) is located on the long arm of chromosome 17. CACNG1 comprises 4 exons and is approximately 12,244 bases long.

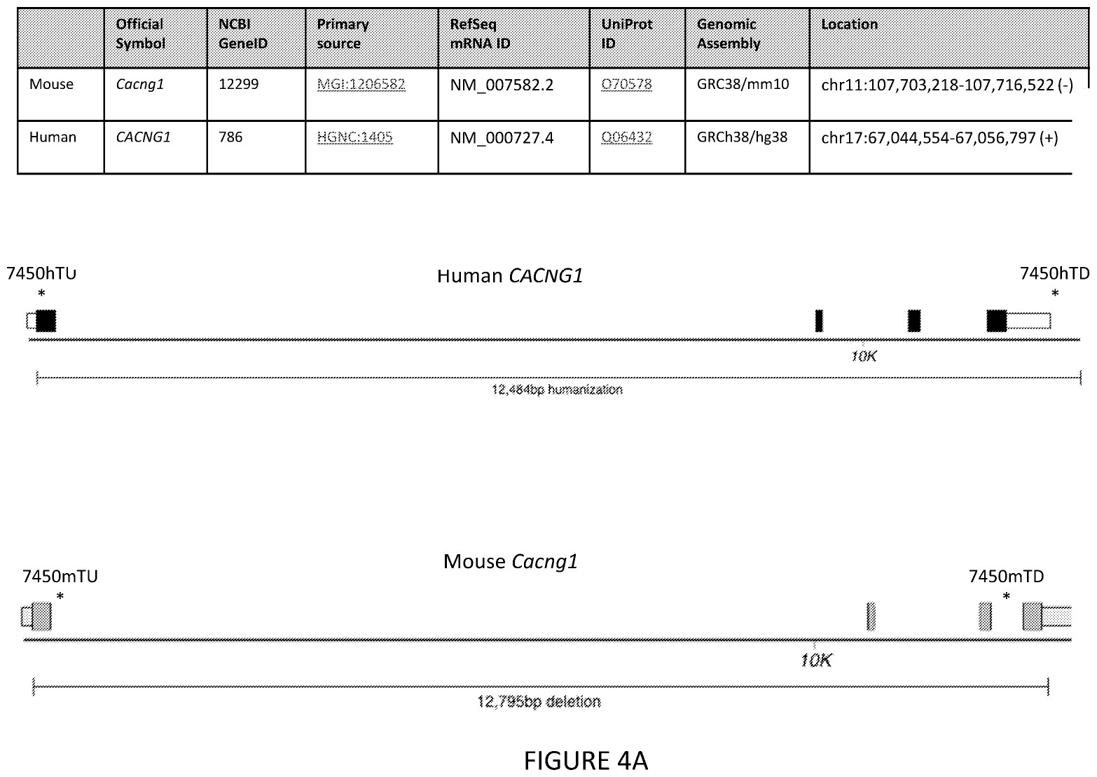

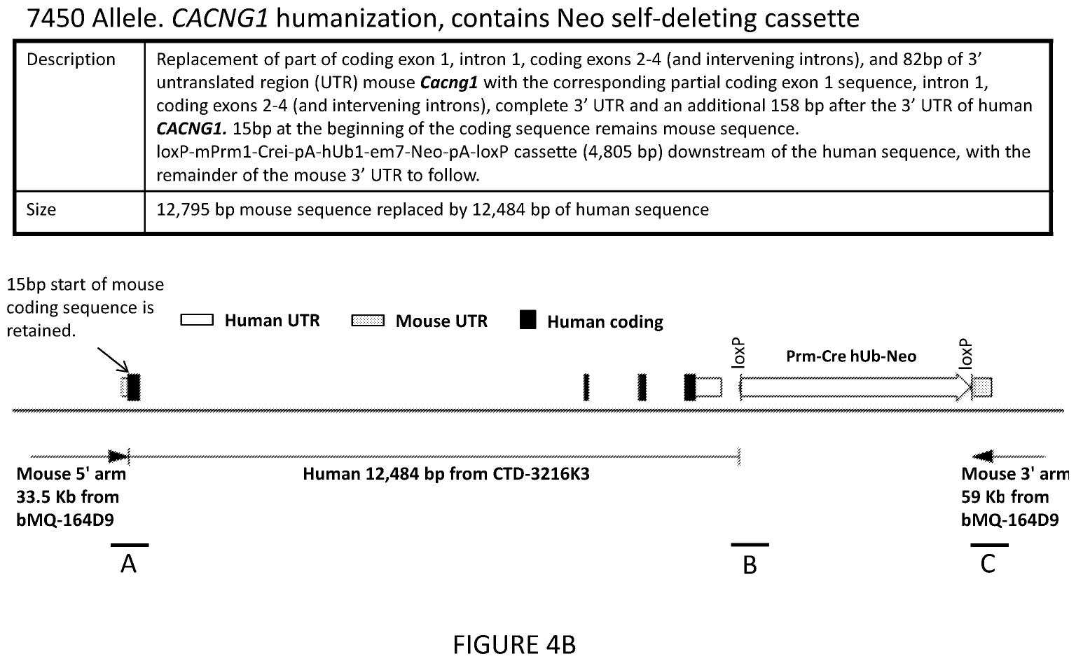

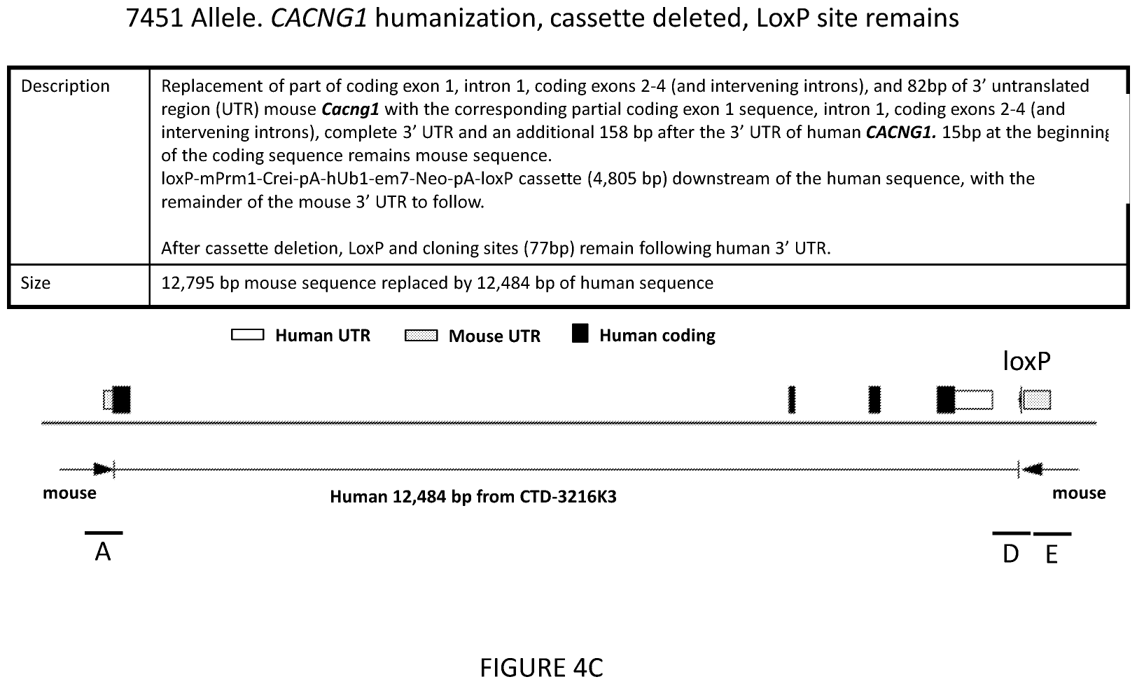

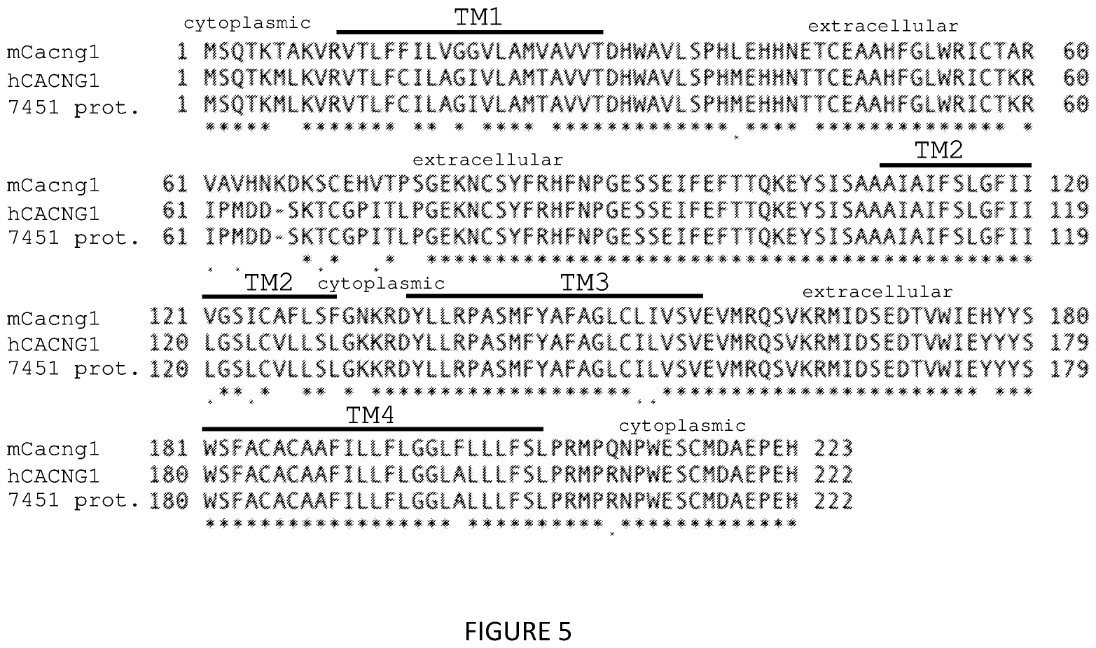

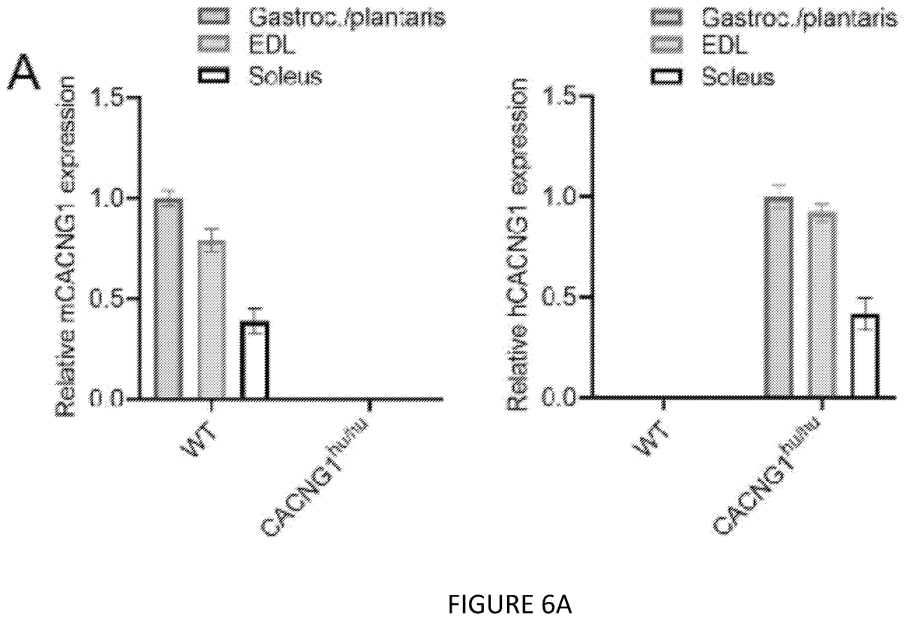

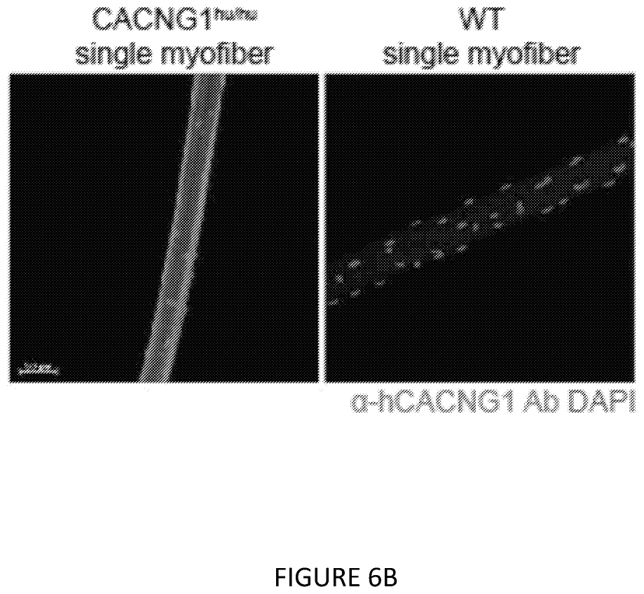

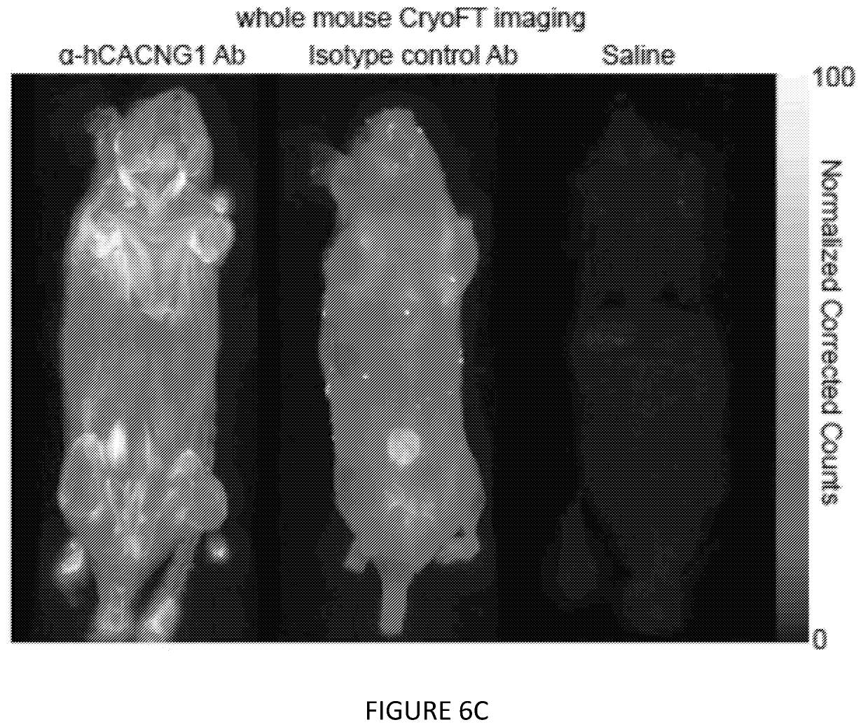

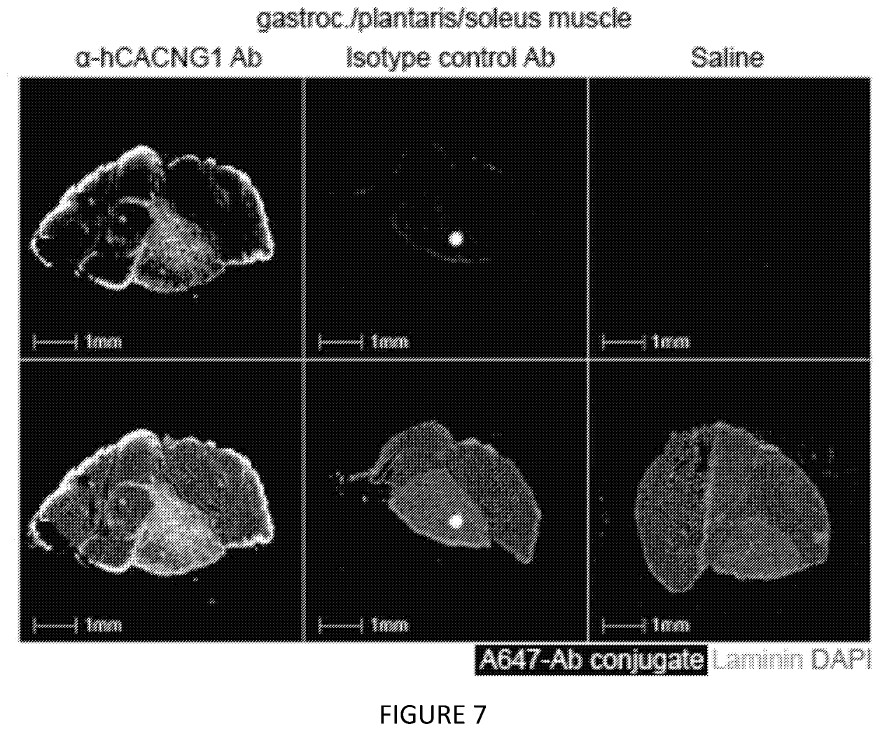

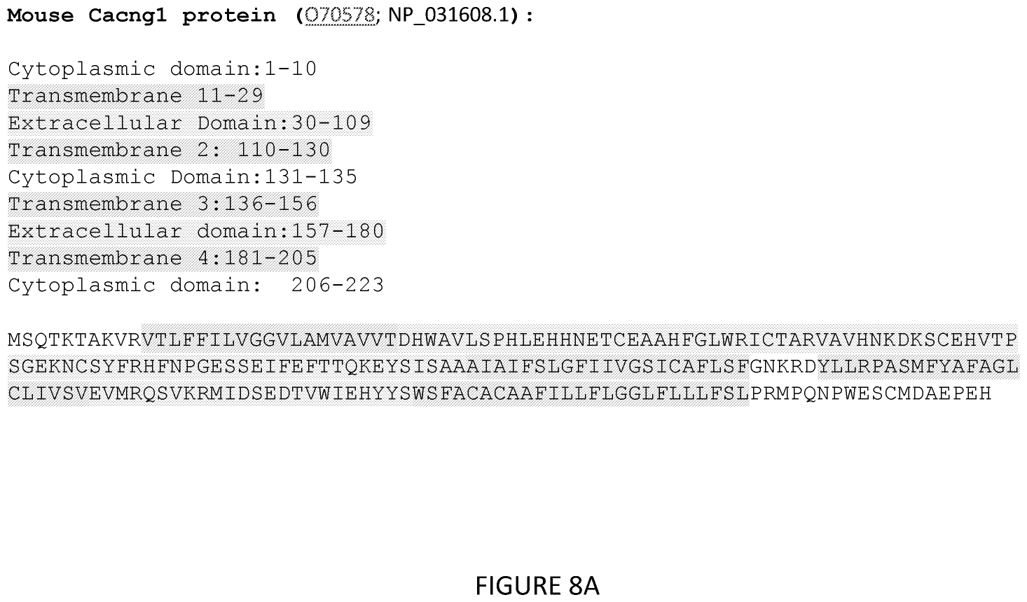

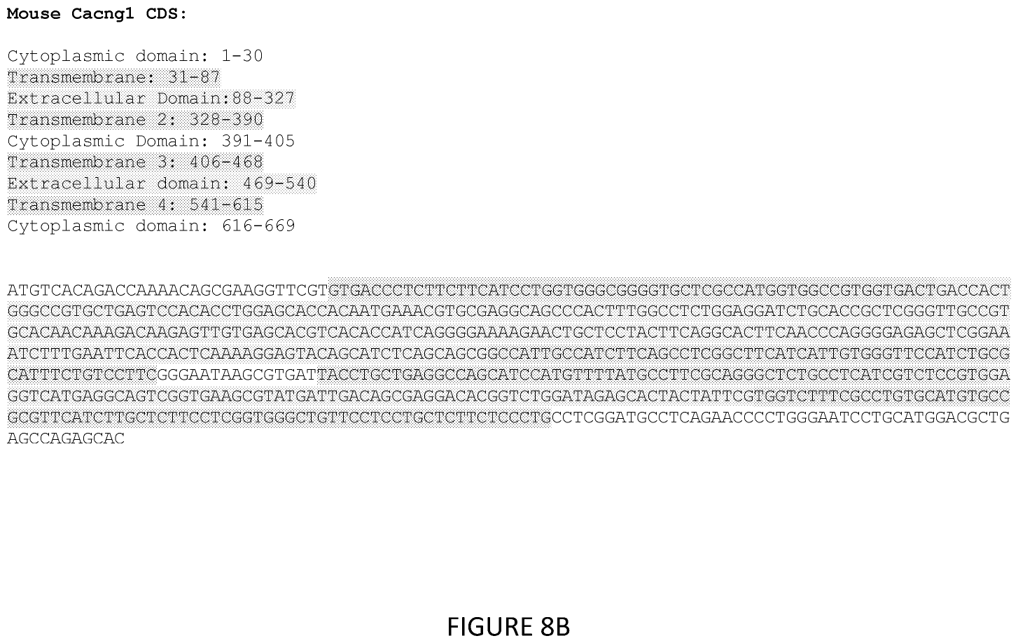

An exampley sequence for human CACNG1 is assigned NCBI Accession Number NM_0007582.2 (See FIG. 4A). An example sequence for mouse Cacng1 is assigned NCBI Accession Number NM_000727.4 (See FIG. 4A). An example human CACNG1 protein is assigned UniProt Accession No. 070578 (See FIG. 4A and FIG. 5). An example mouse CACNG1 protein is assigned UniProt Accession No. Q06432 (See FIG. 4A and FIG. 5). An example human or humanized CACNG1 protein encoded by a modified non-human Cacng1 locus (e.g., 7451) is set forth in FIG. 5. An example rat CACNG1 protein is assigned NCBI Reference Sequence: NP_062128.1. An example orangutan Cacng1 protein is assigned NCBI Reference Sequence: XP_002827789.2.

In some embodiments, the present disclosure provides a non-human animal, a non-human animal cell, or non-human animal genome comprising a nucleic acid sequence encoding a heterologous CACNG1 protein or portion thereof. Such nucleic acid sequences encoding a heterologous CACNG1 protein or portion thereof can comprise: (i) a nucleic acid sequence comprising exon 1 of a human CACNG1 gene or a portion thereof, (ii) a nucleic acid sequence comprising exon 2 of a human CACNG1 gene or a portion thereof, (iii) a nucleic acid sequence comprising exon 3 of a human CACNG1 gene or a portion thereof, (iv) a nucleic acid sequence comprising exon 4 of a human CACNG1 gene or a portion thereof, or (v) any combination of (i)-(iv).

Further, the nucleic acid sequences incorporated into the genomes of a non-human animal, a non-human animal cell, or a non-human animal genome described herein may comprise introns. In some embodiments, the nucleic acid sequence encoding a heterologous CACNG1 protein or portion thereof comprises: (i) a nucleic acid sequence comprising exon 1 of a human CACNG1 gene or a portion thereof, (ii) a nucleic acid sequence of intron 1 of a human CACNG1 gene or a portion thereof, (iii) a nucleic acid sequence comprising exon 2 of a human CACNG1 gene or a portion thereof, (iv) a nucleic acid sequence of intron 2 of a human CACNG1 gene or a portion thereof, (v) a nucleic acid sequence comprising exon 3 of a human CACNG1 gene or a portion thereof, (vi) a nucleic acid sequence of intron 3 of a human CACNG1 gene or a portion thereof, (v) a nucleic acid sequence comprising exon 4 of a human CACNG1 gene or a portion thereof, (vii) a nucleic acid sequence of a 3′ untranslated region (UTR) of a human CACNG1 gene; or (v) any combination of (i)-(iv).

In some embodiments a non-human animal, a non-human animal cell, or a non-human animal genome described herein encodes a humanized coding region for the CACNG1 protein (i.e., some mouse regulatory regions and select human non-coding/coding regions). In some embodiments, the nucleic acid sequence encoding a heterologous CACNG1 protein or portion thereof can comprise, consists essentially of, or consist of a nucleic acid sequence encoding a humanized mouse/human CACNG1 protein, such as the nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28. Any such nucleic acid can be incorporated at an endogenous Cacng1 locus. In some embodiments, a nucleic acid sequence encoding the heterologous CACNG1 protein or portion thereof can replace an orthologous endogenous nucleic acid sequence encoding an endogenous CACNG1 protein or a portion thereof.

In some embodiments, the disclosure provides a non-human animal, a non-human animal cell, or a non-human animal genome wherein the heterologous CACNG1 protein or portion thereof comprises (i) an amino acid sequence set forth as SEQ ID NO:8; (ii) an amino acid sequence set forth as SEQ ID NO: 10; (iii) an amino acid sequence set forth as SEQ ID NO:12; (iv) an amino acid sequence set forth as SEQ ID NO: 14; (v) an amino acid sequence set forth as SEQ ID NO:16; (vi) an amino acid sequence set forth as SEQ ID NO:18; (vii) an amino acid sequence set forth as SEQ ID NO:20; (viii) an amino acid sequence set forth as SEQ ID NO:22; (ix) an amino acid sequence set forth as SEQ ID NO:24; or (x) any combination of (i)-(ii).

### B. Modified Cacng1 Non-Human Animals

The disclosure provides non-human animals with loss-of-function of a CACNG1 protein and chimeric animals (e.g., transgenic rodents expressing a humanized CACNG1 protein). A humanized Cacng1 locus can be an Cacng1 locus in which the entire Cacng1 gene is replaced with the corresponding orthologous human CACNG1 sequence, or it can be an Cacng1 locus in which only a portion of the Cacng1 gene is replaced with the corresponding orthologous human CACNG1 sequence (i.e., humanized). Optionally, the corresponding orthologous human CACNG1 sequence is modified to be codon-optimized based on codon usage in the non-human animal. Replaced (i.e., humanized) regions can include coding regions such as an exon, non-coding regions such as an intron, an untranslated region, or a regulatory region (e.g., a promoter, an enhancer, or a transcriptional repressor-binding element), or any combination thereof. As one example, exons corresponding to 1, 2, 3, 4 or all 4 exons of the human CACNG1 gene can be humanized. For example, exons corresponding to exons 1-4 of the human CACNG1 gene can be humanized. Alternatively, a region of Cacng1 encoding an epitope recognized by an anti-human-CACNG1 antigen-binding protein can be humanized. As another example, one or more or all of the N-terminal cytoplasmic domain, the transmembrane domain, or the intracellular domain can be humanized. For example, all or part of the region of the Cacng1 locus encoding the extracellular domain can be humanized, all or part of the region of the Cacng1 locus encoding the cytoplasmic domain can be humanized, and/or all or part of the region of the Cacng1 locus encoding the transmembrane domain can be humanized. In one example, only all or part of the region of the Cacng1 locus encoding the transmembrane domain is humanized, only all or part of the region of the Cacng1 locus encoding the cytoplasmic domain is humanized, or only all or part of the region of the Cacng1 locus encoding the extracellular region (i.e., the region available as an epitope) is humanized. For example, the regions of the Cacng1 locus encoding the extracellular domain can be humanized such that a chimeric Cacng1 protein is produced with an endogenous N-terminal cytoplasmic domain, an endogenous transmembrane domain, and a humanized trasmembrane domain (epitope). Likewise, introns corresponding to 1, 2, 3, or all 4 introns of the human CACNG1 gene can be humanized. Flanking untranslated regions including regulatory sequences can also be humanized. For example, the 5′ untranslated region (UTR), the 3′UTR, or both the 5′ UTR and the 3′ UTR can be humanized, or the 5′ UTR, the 3′UTR, or both the 5′ UTR and the 3′ UTR can remain endogenous. In one specific example, the 3′ UTR is humanized, but the 5′ UTR remains endogenous. Depending on the extent of replacement by orthologous sequences, regulatory sequences, such as a promoter, can be endogenous or supplied by the replacing human orthologous sequence. For example, the humanized Cacng1 locus can include the endogenous non-human animal Cacng1 promoter.

The Cacng1 protein encoded by the humanized Cacng1 locus can comprise one or more domains that are from a mammalian CACNG1 protein (e.g., human). For example, the Cacng1 protein can comprise one or more or all of a human extracellular domain, a human CACNG1 transmembrane domain, and a human CACNG1 cytoplasmic domain. As one example, the Cacng1 protein can comprise only a human CACNG1 extracellular domain. Optionally, the Cacng1 protein encoded by the humanized Cacng1 locus can also comprise one or more domains that are from the endogenous (i.e., native) non-human animal Cacng1 protein.

Domains from a human CACNG1 protein can be encoded by a fully humanized sequence (i.e., the entire sequence encoding that domain is replaced with the orthologous human CACNG1 sequence) or can be encoded by a partially humanized sequence (i.e., some of the sequence encoding that domain is replaced with the orthologous human CACNG1 sequence, and the remaining endogenous (i.e., native) sequence encoding that domain encodes the same amino acids as the orthologous human CACNG1 sequence such that the encoded domain is identical to that domain in the human CACNG1 protein).

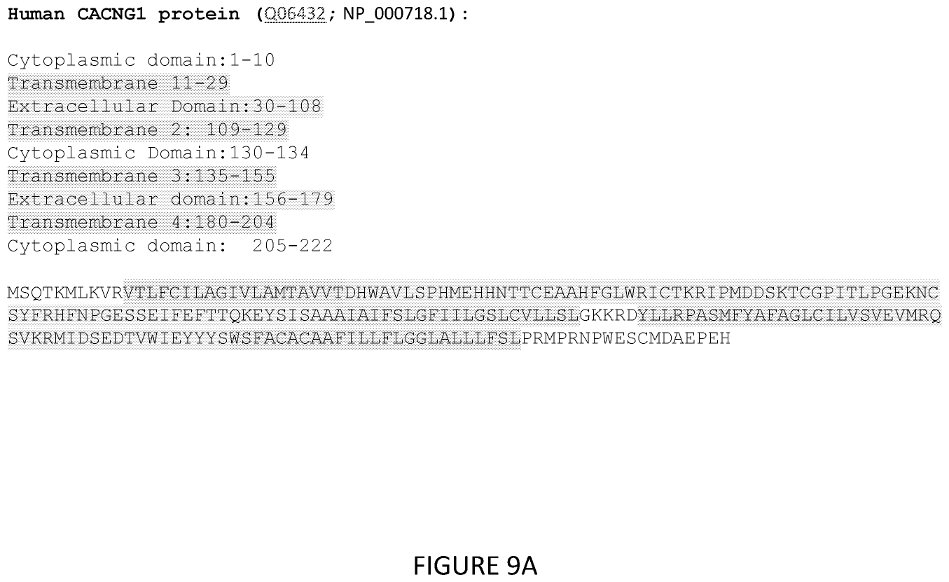

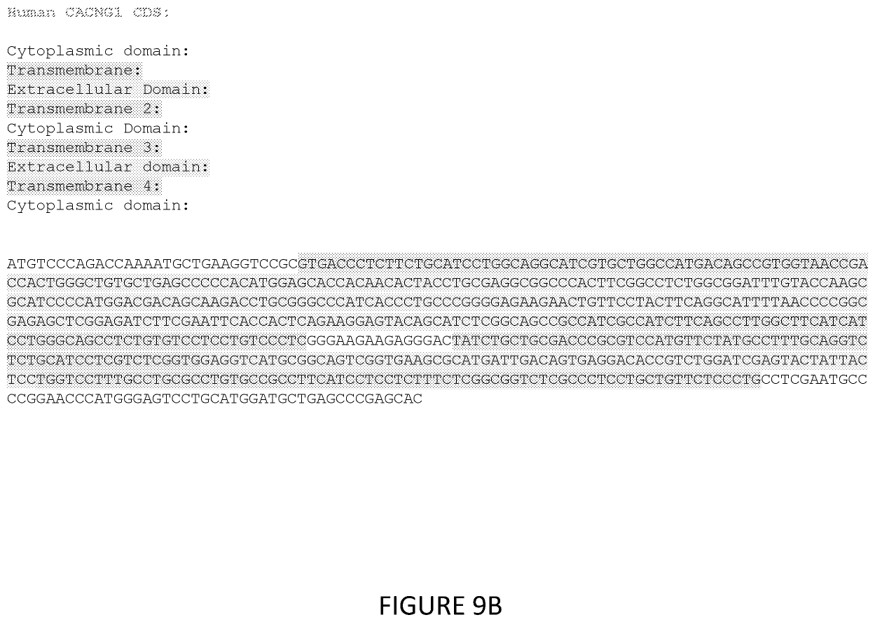

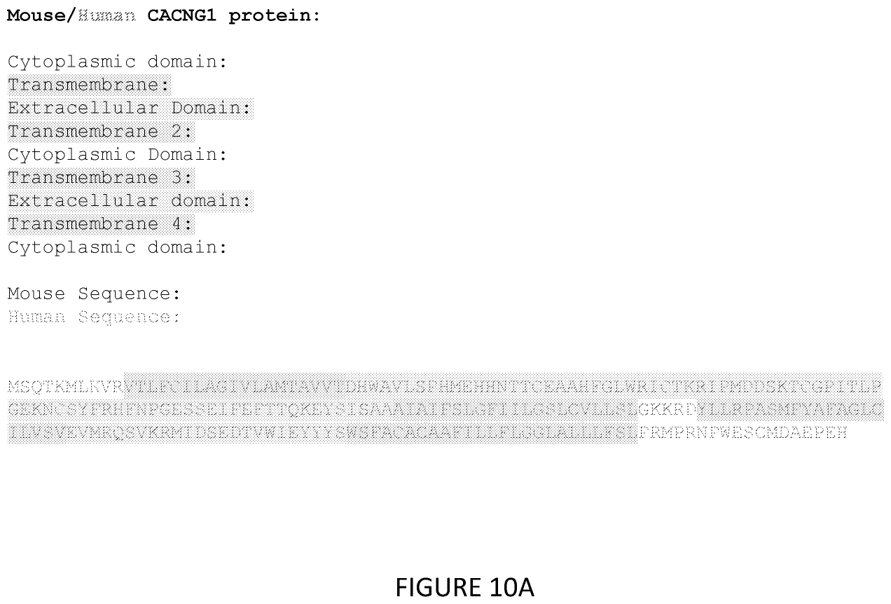

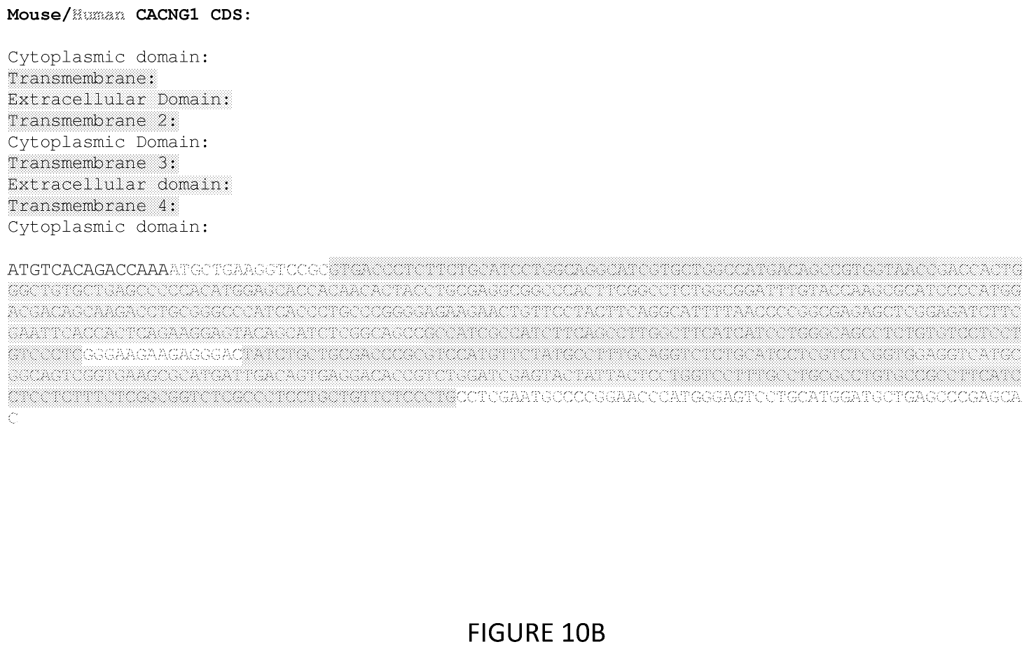

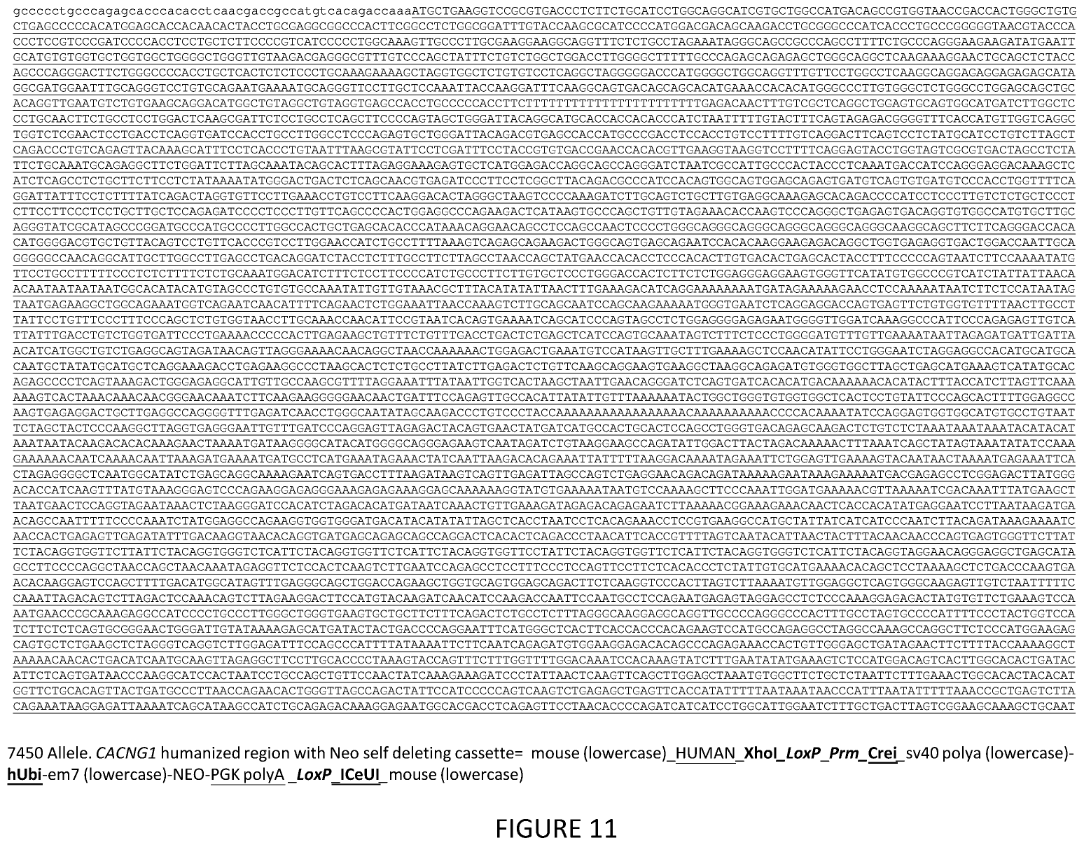

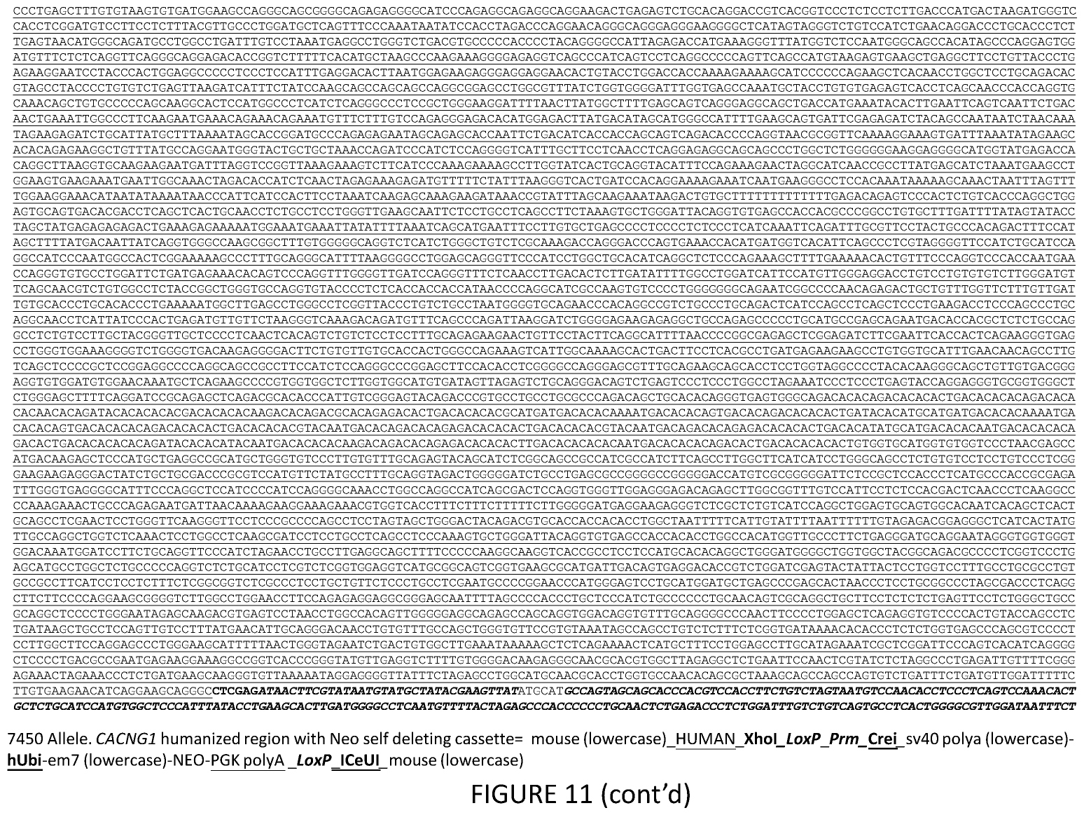

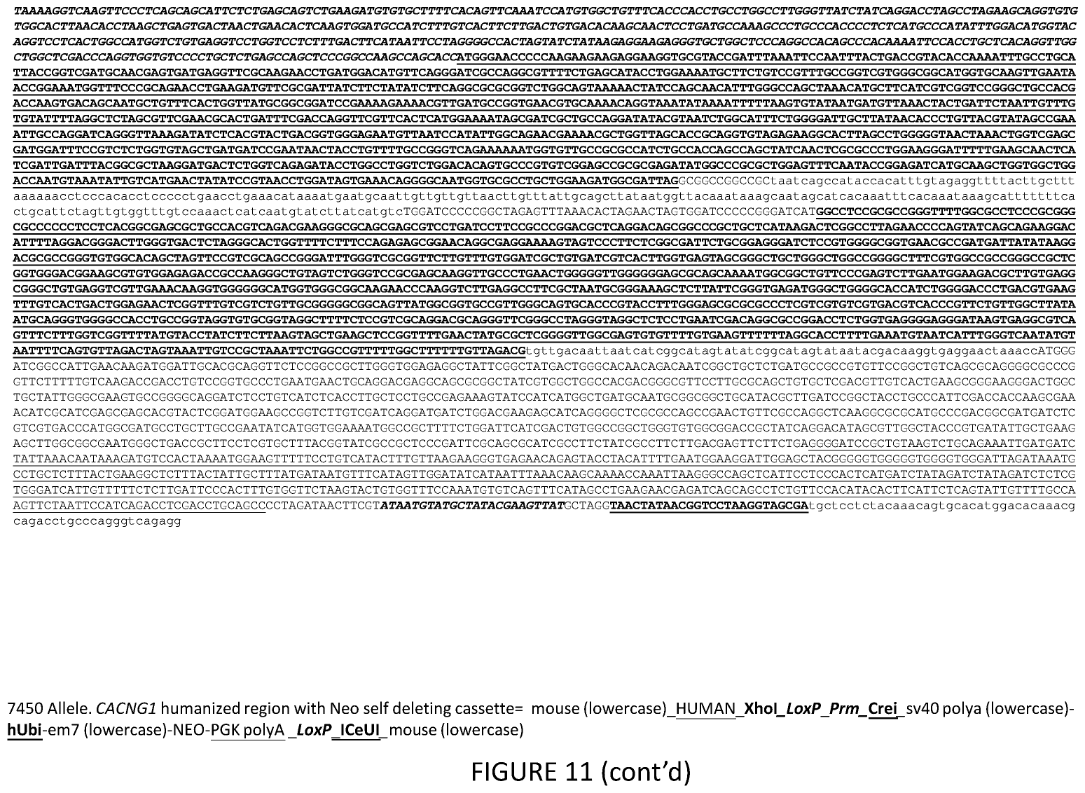

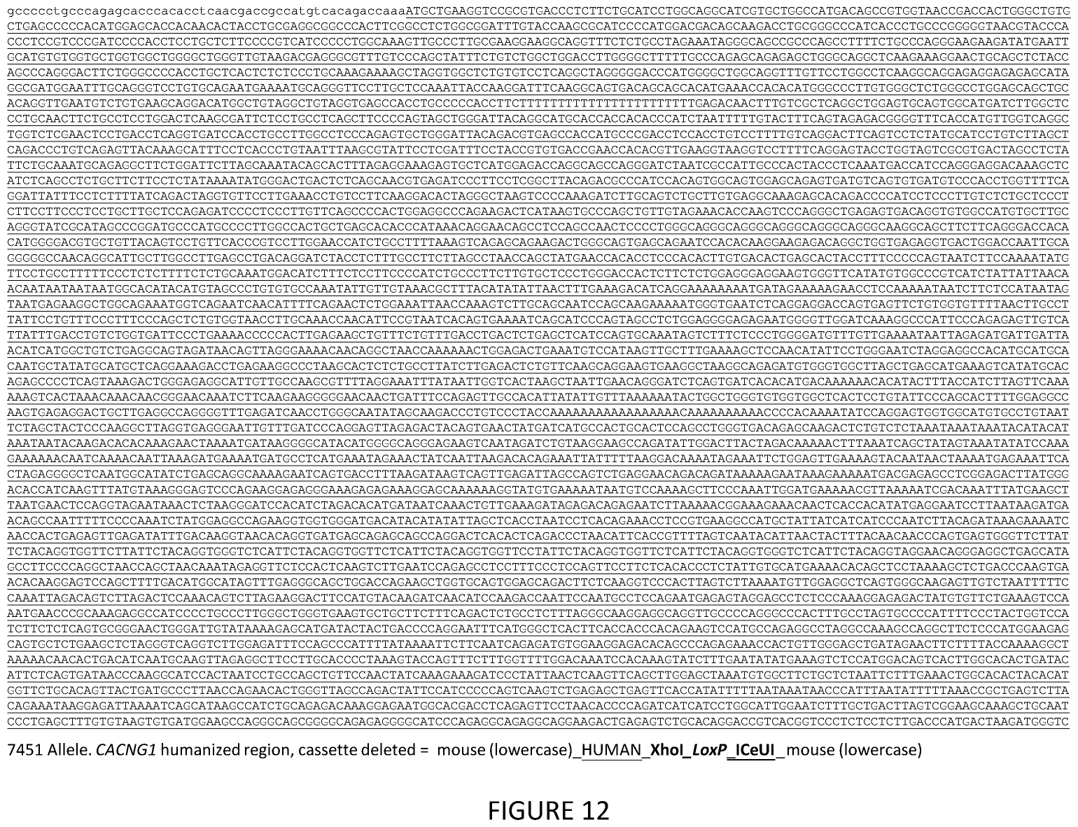

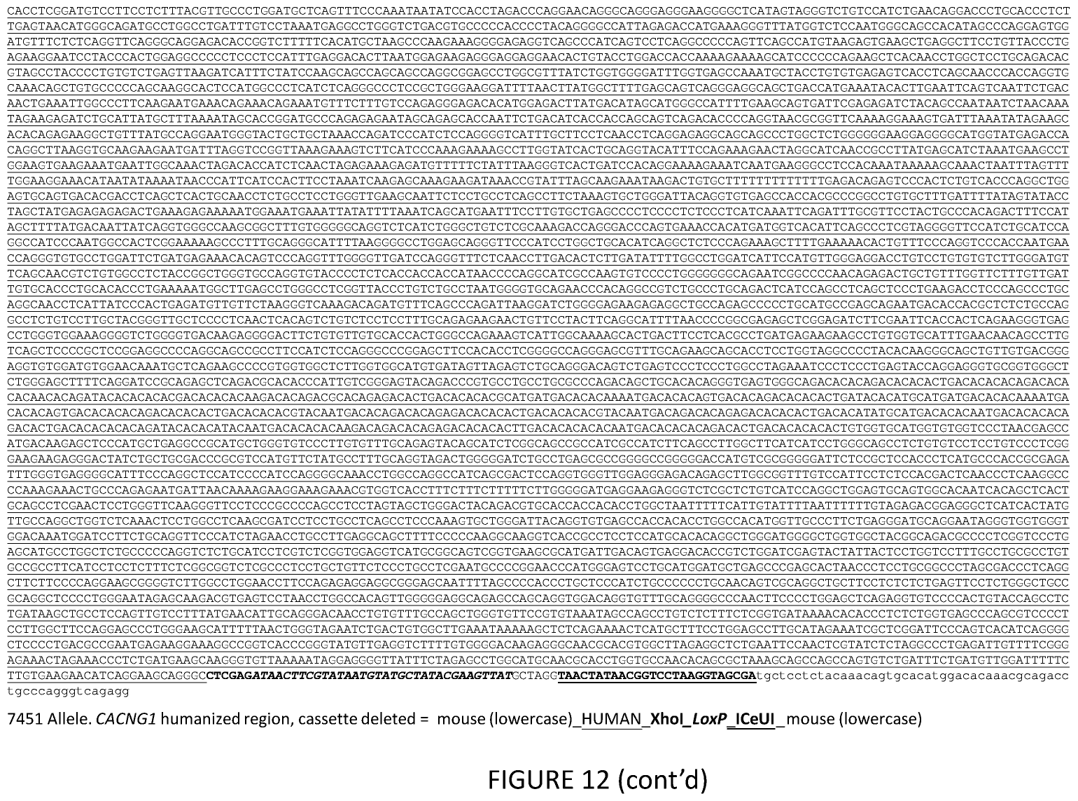

As one example, the Cacng1 protein encoded by the humanized Cacng1 locus can comprise a human CACNG1 extracellular domain (e.g.: human epitope). Optionally, the human CACNG1 transmembrane domain comprises, consists essentially of, or consists of a sequence that is at least 85%, 90%, 95%, 96%, 97%, 98%, 99%, or 100% identical to the sequence shown in FIG. 9A and the CACNG1 protein retains the activity of the native CACNG1 (i.e., retains its function in skeletal muscle).

As another example, the CACNG1 protein encoded by the humanized Cacng1 locus can comprise a human trasnemembrane or cytoplasmic CACNG1 domains. Optionally, the human CACNG1 extracellular domain comprises, consists essentially of, or consists of a sequence that is at least 85%, 90%, 95%, 96%, 97%, 98%, 99%, or 100% identical to FIG. 9A and the Cacng1 protein retains the activity of the native CACNG1.

Optionally, a humanized Cacng1 locus can comprise other elements. Examples of such elements can include selection cassettes, reporter genes, recombinase recognition sites, or other elements. Alternatively, the humanized Cacng1 locus can lack other elements (e.g., can lack a selection marker or selection cassette). Examples of suitable reporter genes and reporter proteins are disclosed elsewhere herein. Examples of suitable selection markers include neomycin phosphotransferase (neoᵣ), hygromycin B phosphotransferase (hygᵣ), puromycin-N-acetyltransferase (puroᵣ), blasticidin S deaminase (bsrᵣ), xanthine/guanine phosphoribosyl transferase (gpt), and herpes simplex virus thymidine kinase (HSV-k). Examples of recombinases include Cre, Flp, and Dre recombinases. One example of a Cre recombinase gene is Crei, in which two exons encoding the Cre recombinase are separated by an intron to prevent its expression in a prokaryotic cell. Such recombinases can further comprise a nuclear localization signal to facilitate localization to the nucleus (e.g., NLS-Crei). Recombinase recognition sites include nucleotide sequences that are recognized by a site-specific recombinase and can serve as a substrate for a recombination event. Examples of recombinase recognition sites include FRT, FRT11, FRT71, attp, att, rox, and lox sites such as loxP, lox511, lox2272, lox66, lox71, loxM2, and lox5171.

Other elements such as reporter genes or selection cassettes can be self-deleting cassettes flanked by recombinase recognition sites. See, e.g., U.S. Pat. No. 8,697,851 and US 2013/0312129, each of which is herein incorporated by reference in its entirety for all purposes. As an example, the self-deleting cassette can comprise a Crei gene (comprises two exons encoding a Cre recombinase, which are separated by an intron) operably linked to a mouse Prm1 promoter and a neomycin resistance gene operably linked to a human ubiquitin promoter. By employing the Prm1 promoter, the self-deleting cassette can be deleted specifically in male germ cells of F0 animals. The polynucleotide encoding the selection marker can be operably linked to a promoter active in a cell being targeted. Examples of promoters are described elsewhere herein. As another specific example, a self-deleting selection cassette can comprise a hygromycin resistance gene coding sequence operably linked to one or more promoters (e.g., both human ubiquitin and EM7 promoters) followed by a polyadenylation signal, followed by a Crei coding sequence operably linked to one or more promoters (e.g., an mPrm1 promoter), followed by another polyadenylation signal, wherein the entire cassette is flanked by loxP sites.

One example humanized Cacng1 locus (e.g., a humanized mouse Cacng1 locus) is one in which coding exons 1-4 are replaced with the corresponding human sequence flanked by a Neo self-deleting cassette. These exons encode the coding domains of Cacng1. Replacement of part of coding exon 1, intron 1, coding exons 2-4 (and intervening introns), and 82 bp of 3′ untranslated region (UTR) mouse Cacng1 with the corresponding partial coding exon 1 sequence, intron 1, coding exons 2-4 (and intervening introns), complete 3′ UTR and an additional 158 bp after the 3′ UTR of human CACNG1, with 15 bp at the beginning of the coding sequence remains mouse sequence provides such non-human animals. See FIG. 4B and FIG. 4C.

### Cacng1^{hu/hu} Mice

The disclosure contemplates cells and non-human animals comprising an exogenous Cacng1 locus. In some embodiments, cells or non-human animals comprising a heterologous Cacng1 locus can express a heterologous CACNG1 protein or a chimeric CACNG1 protein in which one or more fragments of the native Cacng1 protein have been replaced with corresponding fragments from the heterologous CACNG1 sequence (e.g., all or part of the extracellular domain; all of the CACNG1 codin region).

In some embodiments, cells and non-human animals disclosed herein comprise an exogenous nucleic acid sequence encoding, of a human CACNG1 protien, amino acids 1-10, amino acids 11-29, amino acids 30-108, amino acids 109-129, amino acids 130-134, amino acids 135-155, amino acids 156-179, amino acids 180-204, amino acids 205-222, and/or combinations thereof.

### Loss of Function Cacng1^{-/-} Mice

CACNG1^{-/-} mice were generated with gene editing techniques to determine whether deletion of CACNG1 affects skeletal muscle mass or function. Because some of the non-human animals described herein lack a Cacng1 locus, such non-human animals can provide an understanding of the impact of loss-of-function on the Cacng1 protein in a holistic manner.

In some embodiments, the disclosure provides a non-human animal, non-human animal cell, or non-human animal genome comprising a knockout mutation of an endogenous Cacng1 gene. In some embodiments, such knockout mutations can comprise a deletion of the Cacng1 gene or a portion thereof. In some cases, the knockout mutation can comprise a deletion of the entire coding sequence of the Cacng1 gene. In some embodiments, the Cacng1^{-/-} human animal genome does not express any CACNG1 protein.

In some embodiments, the non-human animal, non-human animal cell, or non-human animal genome does not exhibit any gross mutant phenotype (i.e., does not present any measurable trait, particularly muscle strength, structure, or a functional trait, that is statistically significant from a wild-type counterpart).

### C. Non-Human Cells and Non-Human Animals Comprising a Heterologous Cacng1 Locus

Non-human animal cells and non-human animals comprising a humanized Cacng1 locus as described herein are provided. The cells or non-human animals can be heterozygous or homozygous for the humanized Cacng1 locus. A diploid organism has two alleles at each genetic locus. Each pair of alleles represents the genotype of a specific genetic locus. Genotypes are described as homozygous if there are two identical alleles at a particular locus and as heterozygous if the two alleles differ.

The non-human animal cells provided herein can be, for example, any non-human cell comprising an Cacng1 locus or a genomic locus homologous or orthologous to the human CACNG1 locus. The cells can be eukaryotic cells, which include, for example, fungal cells (e.g., yeast), plant cells, animal cells, mammalian cells, non-human mammalian cells, and human cells. An animal can be, for example, a mammal, fish, or bird. A mammalian cell can be, for example, a non-human mammalian cell, a rodent cell, a rat cell, a mouse cell, or a hamster cell. Other non-human mammals include, for example, non-human primates, monkeys, apes, orangutans, cats, dogs, rabbits, horses, bulls, deer, bison, livestock (e.g., bovine species such as cows, steer, and so forth; ovine species such as sheep, goats, and so forth; and porcine species such as pigs and boars). Birds include, for example, chickens, turkeys, ostrich, geese, ducks, and so forth. Domesticated animals and agricultural animals are also included. The term “non-human” excludes humans.

The cells can also be any type of undifferentiated or differentiated state. For example, a cell can be a totipotent cell, a pluripotent cell (e.g., a human pluripotent cell or a non-human pluripotent cell such as a mouse embryonic stem (ES) cell or a rat ES cell), or a non-pluripotent cell. Totipotent cells include undifferentiated cells that can give rise to any cell type, and pluripotent cells include undifferentiated cells that possess the ability to develop into more than one differentiated cell types. Such pluripotent and/or totipotent cells can be, for example, ES cells or ES-like cells, such as an induced pluripotent stem (iPS) cells. ES cells include embryo-derived totipotent or pluripotent cells that can contribute to any tissue of the developing embryo upon introduction into an embryo. ES cells can be derived from the inner cell mass of a blastocyst and can differentiate into cells of any of the three vertebrate germ layers (endoderm, ectoderm, and mesoderm).

The cells provided herein can also be germ cells (e.g., sperm or oocytes). The cells can be mitotically competent cells or mitotically-inactive cells, meiotically competent cells or meiotically-inactive cells. Similarly, the cells disclosed herein can also be primary somatic cells or cells that are not a primary somatic cell. Somatic cells include any cell that is not a gamete, germ cell, gametocyte, or undifferentiated stem cell. For example, the cells disclosed herein can be muscle cells, such as skeletal muscle cells.

Suitable cells provided herein also include primary cells. Primary cells include cells or cultures of cells that have been isolated directly from an organism, organ, or tissue. Primary cells include cells that are neither transformed nor immortal. Primary cells include any cell obtained from an organism, organ, or tissue which was not previously passed in tissue culture or has been previously passed in tissue culture but is incapable of being indefinitely passed in tissue culture. Such cells can be isolated by conventional techniques and include, for example, muscle cells (e.g., skeletal mucle cells).

Other suitable cells provided herein include immortalized cells. Immortalized cells include cells from a multicellular organism that would normally not proliferate indefinitely but, due to mutation or alteration, have evaded normal cellular senescence and instead can keep undergoing division. Such mutations or alterations can occur naturally or be intentionally induced. Examples of immortalized cell lines are myofiber cell lines. Immortalized or primary cells include cells that can be used for culturing or for expressing recombinant genes or proteins.

The cells provided herein also include one-cell stage embryos (i.e., fertilized oocytes or zygotes). Such one-cell stage embryos can be from any genetic background (e.g., BALB/c, C57BL/6, 129, or a combination thereof for mice), can be fresh or frozen, and can be derived from natural breeding or in vitro fertilization.

The cells provided herein can be normal, healthy cells, or can be diseased or mutant-bearing cells.

Non-human animals comprising a humanized Cacng1 locus as described herein can be made by the methods described elsewhere herein. An animal can be, for example, a mammal, fish, or bird. Non-human mammals include, for example, non-human primates, monkeys, apes, orangutans, cats, dogs, horses, bulls, deer, bison, sheep, rabbits, rodents (e.g., mice, rats, hamsters, and guinea pigs), and livestock (e.g., bovine species such as cows and steer; ovine species such as sheep and goats; and porcine species such as pigs and boars). Birds include, for example, chickens, turkeys, ostrich, geese, and ducks. Domesticated animals and agricultural animals are also included. The term “non-human animal” excludes humans. Preferred non-human animals include, for example, rodents, such as mice and rats.

The non-human animals can be from any genetic background. For example, suitable mice can be from a 129 strain, a C57BL/6 strain, a mix of 129 and C57BL/6, a BALB/c strain, or a Swiss Webster strain. Examples of 129 strains include 129P1, 129P2, 129P3, 129X1, 129S1 (e.g., 12951/SV, 12951/Svlm), 129S2, 129S4, 129S5, 12959/SvEvH, 129S6 (129/SvEvTac), 129S7, 129S8, 129T1, and 129T2. See, e.g., Festing et al. (1999) Mammalian Genome 10:836, herein incorporated by reference in its entirety for all purposes. Examples of C57BL strains include C57BL/A, C57BL/An, C57BL/GrFa, C57BL/Kal_wN, C57BL/6, C57BL/6J, C57BL/6ByJ, C57BL/6NJ, C57BL/10, C57BL/10ScSn, C57BL/10Cr, and C57BL/Ola. Suitable mice can also be from a mix of an aforementioned 129 strain and an aforementioned C57BL/6 strain (e.g., 50% 129 and 50% C57BL/6). Likewise, suitable mice can be from a mix of aforementioned 129 strains or a mix of aforementioned BL/6 strains (e.g., the 12956 (129/SvEvTac) strain).

Similarly, rats can be from any rat strain, including, for example, an ACI rat strain, a Dark Agouti (DA) rat strain, a Wistar rat strain, a LEA rat strain, a Sprague Dawley (SD) rat strain, or a Fischer rat strain such as Fisher F344 or Fisher F6. Rats can also be obtained from a strain derived from a mix of two or more strains recited above. For example, a suitable rat can be from a DA strain or an ACI strain. The ACI rat strain is characterized as having black agouti, with white belly and feet and an RT1^{av1} haplotype. Such strains are available from a variety of sources including Harlan Laboratories. The Dark Agouti (DA) rat strain is characterized as having an agouti coat and an RT1^{av1} haplotype. Such rats are available from a variety of sources including Charles River and Harlan Laboratories. Some suitable rats can be from an inbred rat strain. See, e.g., US 2014/0235933, herein incorporated by reference in its entirety for all purposes.

### III. Methods of Making Non-Human Animals Comprising a Heterologous Cacng1 Locus

Various methods are provided for making a non-human animal comprising a heterologous Cacng1 locus as disclosed elsewhere herein. Any convenient method or protocol for producing a genetically modified organism is suitable for producing such a genetically modified non-human animal. See, e.g., Cho et al. (2009) Current Protocols in Cell Biology 42:19.11:19.11.1-19.11.22 and Gama Sosa et al. (2010) Brain Struct. Funct. 214(2-3):91-109, each of which is herein incorporated by reference in its entirety for all purposes. Such genetically modified non-human animals can be generated, for example, through gene knock-in at a targeted Cacng1 locus.

For example, the method of producing a non-human animal comprising a humanized Cacng1 locus can comprise: (1) modifying the genome of a pluripotent cell to comprise the humanized Cacng1 locus; (2) identifying or selecting the genetically modified pluripotent cell comprising the humanized Cacng1 locus; (3) introducing the genetically modified pluripotent cell into a non-human animal host embryo cells in vitro; and (4) implanting and gestating the host embryo cells in a surrogate mother. Optionally, the host embryo comprising modified pluripotent cell (e.g., a non-human ES cell) can be incubated until the blastocyst stage before being implanted into and gestated in the surrogate mother to produce an F0 non-human animal. The surrogate mother can then produce an F0 generation non-human animal comprising the humanized Cacng1 locus.

The methods can further comprise identifying a cell or animal having a modified target genomic locus. Various methods can be used to identify cells and animals having a targeted genetic modification.

The screening step can comprise, for example, a quantitative assay for assessing modification of allele (MOA) of a parental chromosome. For example, the quantitative assay can be carried out via a quantitative PCR, such as a real-time PCR (qPCR). The real-time PCR can utilize a first primer set that recognizes the target locus and a second primer set that recognizes a non-targeted reference locus. The primer set can comprise a fluorescent probe that recognizes the amplified sequence.

Other examples of suitable quantitative assays include fluorescence-mediated in situ hybridization (FISH), comparative genomic hybridization, isothermic DNA amplification, quantitative hybridization to an immobilized probe(s), INVADER® Probes, TAQMAN® Molecular Beacon probes, or ECLIPSE™ probe technology (see, e.g., US 2005/0144655, incorporated herein by reference in its entirety for all purposes).

An example of a suitable pluripotent cell is an embryonic stem (ES) cell (e.g., a mouse ES cell or a rat ES cell). The modified pluripotent cell can be generated, for example, through recombination by (a) introducing into the cell one or more targeting vectors comprising an insert nucleic acid flanked by 5′ and 3′ homology arms corresponding to 5′ and 3′ target sites, wherein the insert nucleic acid comprises a heterologous Cacng1 locus; and (b) identifying at least one cell comprising in its genome the insert nucleic acid integrated at the target genomic locus. Alternatively, the modified pluripotent cell can be generated by (a) introducing into the cell: (i) a nuclease agent, wherein the nuclease agent induces a nick or double-strand break at a recognition site within the target genomic locus; and (ii) one or more targeting vectors comprising an insert nucleic acid flanked by 5′ and 3′ homology arms corresponding to 5′ and 3′ target sites located in sufficient proximity to the recognition site, wherein the insert nucleic acid comprises the heterologous Cacng1 locus; and (c) identifying at least one cell comprising a modification (e.g., integration of the insert nucleic acid) at the target genomic locus. Any nuclease agent that induces a nick or double-strand break into a desired recognition site can be used. Examples of suitable nucleases include a Transcription Activator-Like Effector Nuclease (TALEN), a zinc-finger nuclease (ZFN), a meganuclease, and Clustered Regularly Interspersed Short Palindromic Repeats (CRISPR)/CRISPR-associated (Cas) systems or components of such systems (e.g., CRISPR/Cas9). See, e.g., US 2013/0309670 and US 2015/0159175, each of which is herein incorporated by reference in its entirety for all purposes.

The donor cell can be introduced into a host embryo at any stage, such as the blastocyst stage or the pre-morula stage (i.e., the 4 cell stage or the 8 cell stage). Progeny that are capable of transmitting the genetic modification though the germline are generated. See, e.g., U.S. Pat. No. 7,294,754, herein incorporated by reference in its entirety for all purposes.

Alternatively, the method of producing the non-human animals described elsewhere herein can comprise: (1) modifying the genome of a one-cell stage embryo to comprise the heterologous Cacng1 locus using the methods described above for modifying pluripotent cells; (2) selecting the genetically modified embryo; and (3) implanting and gestating the genetically modified embryo into a surrogate mother. Progeny that are capable of transmitting the genetic modification though the germline are generated.

Nuclear transfer techniques can also be used to generate the non-human mammalian animals. Briefly, methods for nuclear transfer can include the steps of: (1) enucleating an oocyte or providing an enucleated oocyte; (2) isolating or providing a donor cell or nucleus to be combined with the enucleated oocyte; (3) inserting the cell or nucleus into the enucleated oocyte to form a reconstituted cell; (4) implanting the reconstituted cell into the womb of an animal to form an embryo; and (5) allowing the embryo to develop. In such methods, oocytes are generally retrieved from deceased animals, although they may be isolated also from either oviducts and/or ovaries of live animals. Insertion of the donor cell or nucleus into the enucleated oocyte to form a reconstituted cell can be by microinjection of a donor cell under the zona pellucida prior to fusion. Fusion may be induced by application of a DC electrical pulse across the contact/fusion plane (electrofusion), by exposure of the cells to fusion-promoting chemicals, such as polyethylene glycol, or by way of an inactivated virus, such as the Sendai virus. A reconstituted cell can be activated by electrical and/or non-electrical means before, during, and/or after fusion of the nuclear donor and recipient oocyte. Activation methods include electric pulses, chemically induced shock, penetration by sperm, increasing levels of divalent cations in the oocyte, and reducing phosphorylation of cellular proteins (as by way of kinase inhibitors) in the oocyte. The activated reconstituted cells, or embryos, can be cultured in media and then transferred to the womb of an animal. See, e.g., US 2008/0092249, WO 1999/005266, US 2004/0177390, WO 2008/017234, and U.S. Pat. No. 7,612,250, each of which is herein incorporated by reference in its entirety for all purposes.

The various methods provided herein allow for the generation of a genetically modified non-human F0 animal wherein the cells of the genetically modified F0 animal comprise the humanized Cacng1 locus. It is recognized that depending on the method used to generate the F0 animal, the number of cells within the F0 animal that have the heterologous Cacng1 locus will vary. The introduction of the donor ES cells into a pre-morula stage embryo from a corresponding organism (e.g., an 8-cell stage mouse embryo) via for example, the VELOCIMOUSE® method allows for a greater percentage of the cell population of the F0 animal to comprise cells having the nucleotide sequence of interest comprising the targeted genetic modification. For example, at least 50%, 60%, 65%, 70%, 75%, 85%, 86%, 87%, 87%, 88%, 89%, 90%, 91%, 92%, 93%, 94%, 95%, 96%, 97%, 98%, 99% or 100% of the cellular contribution of the non-human F0 animal can comprise a cell population having the targeted modification.

The cells of the genetically modified F0 animal can be heterozygous for the heterologous Cacng1 locus or can be homozygous for the heterologous Cacng1 locus.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or embodiment of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

In some embodiments, the disclosure provides a method of making a non-human animal, a non-human animal cell, or a non-human animal genome of described herein, comprising inserting the nucleic acid sequence encoding the heterologous CACNG1 protein or portion thereof into the genome of the non-human animal, the genome of the non-human animal cell, or the non-human animal genome.

A variety of chimeric nucleic acids can be specifically used for such purposes. In some embodiments, a chimeric nucleic acid molecule that encodes a functional CACNG1 protein comprising a nucleic acid sequence of a modified non-human animal Cacng1 gene, wherein the modified non-human animal Cacng1 gene comprises a replacement of a nucleic sequence encoding a portion of the non-human animal CACNG1 protein with a homologous nucleic acid sequence encoding a heterologous CACNG1 protein or portion thereof can be used in the genetic editing of a cell or genome described herein. In some cases, such chimeric nucleic acid molecules comprise (i) a nucleic acid sequence comprising exon 1 of a human CACNG1 gene or a portion thereof, (ii) a nucleic acid sequence of intron 1 of a human CACNG1 gene or a portion thereof, (iii) a nucleic acid sequence comprising exon 2 of a human CACNG1 gene or a portion thereof, (iv) a nucleic acid sequence of intron 2 of a human CACNG1 gene or a portion thereof, (v) a nucleic acid sequence comprising exon 3 of a human CACNG1 gene or a portion thereof, (vi) a nucleic acid sequence of intron 3 of a human CACNG1 gene or a portion thereof, (v) a nucleic acid sequence comprising exon 4 of a human CACNG1 gene or a portion thereof; (vii) a nucleic acid sequence of a 3′ untranslated region (UTR) of a human CACNG1 gene; or (v) any combination of (i)-(iv). In some isntances, the chimeric molecule provides a drug selection cassette.

In some embodiments, a chimeric nucleic acid molecule described herein comprises (i) a 5′ homology arm upstream of the modified non-human animal Cacng1 gene and (ii) a 3′ homology arm downstream of the modified non-human animal Cacng1 gene. In some embodiments, the 5′ homology arm and 3′ homology arm are configured to undergo homologous recombination with a non-human animal Cacng1 locus of interest, and following homologous recombination with a non-human animal Cacng1 locus of interest, the modified Cacng1 gene replaces the non-human animal Cacng1 gene at the non-human animal Cacng1 locus of interest and is operably linked to an endogenous promoter that drives expression of the non-human animal Cacng1 gene at the non-human animal Cacng1 locus of interest. In some embodiments, the chimeric nucleic acid molecule comprises (i) the 5′ homology arm comprises a nucleic acid sequence set forth as SEQ ID NO: 25 and/or (ii) the 3′ homology arm comprises a nucleic acid sequence set forth as SEQ ID NO:26. In some embodiments, the chimeric nucleic acid molecule comprises the nucleic acid sequence comprises a nucleic acid sequence set forth as SEQ ID NO:6.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequences follow the standard convention of beginning at the amino terminus of the sequence and proceeding forward (i.e., from left to right in each line) to the carboxy terminus.

TABLE 1
Description of Sequences.

| SEQ | | |
|---|---|---|
| ID | | |
| NO | Type | Description |
| 1 | DNA | mCacng1-Coding Sequence- |
| | | NCBI Gene ID 12299 |
| 2 | Protein | mCACNG1-UniProt ID 070578 |
| 3 | DNA | hCACNG1 Coding Sequence- |
| | | NCBI Gene ID 786 |
| 4 | Protein | hCACNG1-UniProt ID 070578 |
| 5 | DNA | human portion of mouse/ |
| | | human CACNG1 cds |
| 6 | DNA | mouse/human CACNG1 cds |
| 7 | Protein | amino acid sequence of human CYT1 |
| 8 | DNA | nucleic acid sequence of human CYT1 |
| 9 | Protein | amino acid sequence of human TM1 |
| 10 | DNA | nucleic acid sequence of human TM1 |
| 11 | Protein | amino acid sequence of human EX1 |
| 12 | DNA | nucleic acid sequence of human EX1 |
| 13 | Protein | amino acid sequence of human TM2 |
| 14 | DNA | nucleic acid sequence of human TM2 |
| 15 | Protein | amino acid sequence of human CYT2 |
| 16 | DNA | nucleic acid sequence of human CYT2 |
| 17 | Protein | amino acid sequence of human TM3 |
| 18 | DNA | nucleic acid sequence of human TM3 |
| 19 | Protein | amino acid sequence of human EX2 |
| 20 | DNA | nucleic acid sequence of human EX2 |
| 21 | Protein | amino acid sequence of human TM4 |
| 22 | DNA | nucleic acid sequence of human TM4 |
| 23 | Protein | amino acid sequence of human CYT3 |
| 24 | DNA | nucleic acid sequence of |
| | | human CYT3 |
| 25 | DNA | mouse 5′ arm |
| 26 | DNA | mouse 3′ arm |
| 27 | DNA | 7450 allele |
| 28 | DNA | 7451 allele |
| 29 | DNA | mCacng1 locus knockout |
| | | allele (6866) |
| 30 | DNA | hCACNG1: fwd- |
| | | GGCGAGAGCTCGGAGATC |
| 31 | DNA | hCACNG1: rev- |
| | | GGCTGCCCAGGATGATGAAG |
| 32 | DNA | hCACNG1: probe- |
| | | TCGAATTCACCACTCAGAAGGAGTACA |
| 33 | DNA | mCACNG1: fwd- |
| | | CCGTGCACAACAAAGACAAGAG |
| 34 | DNA | mCACNG1: rev- |
| | | GGCTGCCCAGGATGATGAAG |
| 35 | DNA | mCACNG1: probe- |
| | | TGTGAGCACGTCACACCATCAGG |

### EXAMPLES

### Example 1. CACNG1 is Specifically Expressed in Skeletal Muscle

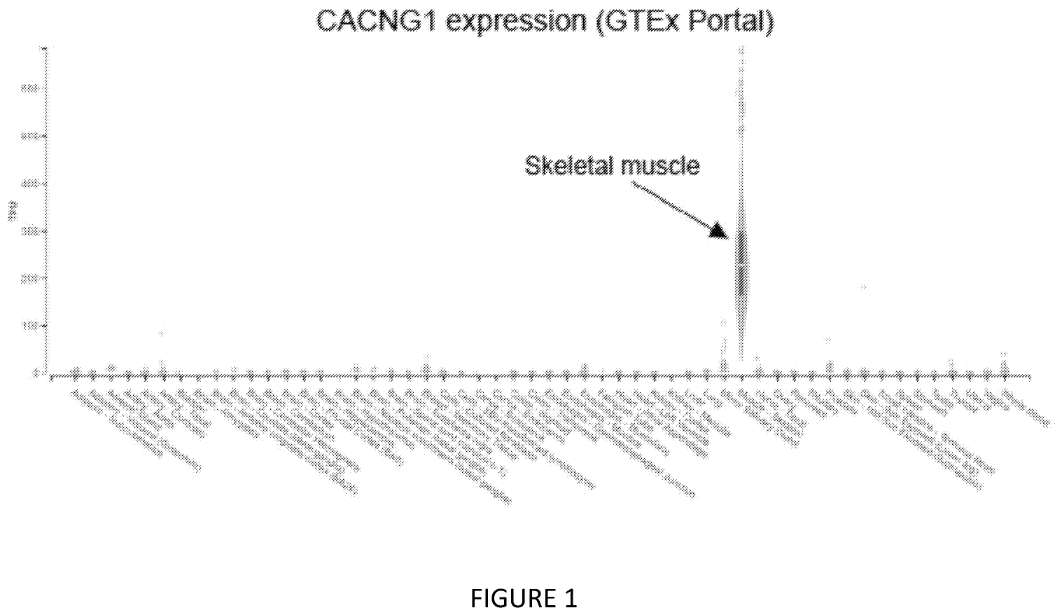

The skeletal muscle dihydropyridine receptor (DHPR) is an L-type calcium channel that is involved in excitation-contraction coupling. The skeletal muscle DHPR consists of 5 subunits, with the α1s subunit playing a critical role in muscle contraction via its physical interaction with the ryanodine receptor to regulate calcium release from the sarcoplasmic reticulum. The γ1 subunit (CACNG1) was found to be highly and specifically expressed in skeletal muscle (FIG. 1A). Thus, humanized Cacng1 were generated mice for use in validation of liver-specific delivery of different therapeutics utilizing a number of different approaches.

### Example 2. Generation and Analysis of CACNG1 Knockout Mice (CACNG1^{-/-})

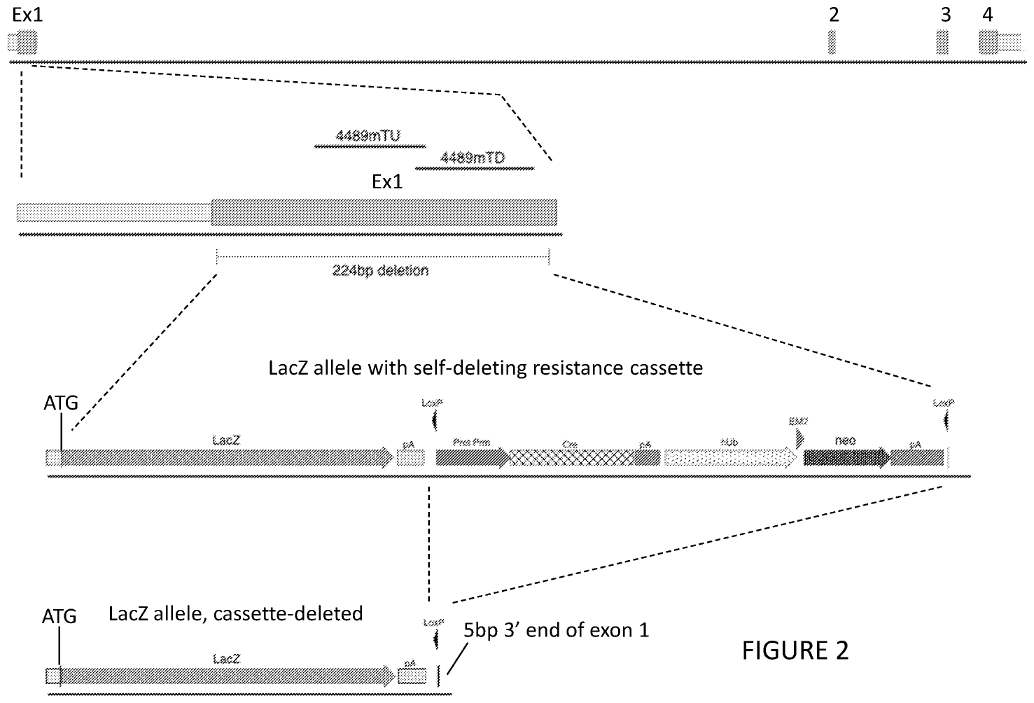

CACNG1^{-/-} mice were generated and bred in-house to determine whether deletion of CACNG1 affects skeletal muscle mass or function. The Cacng1 ablation construct was designed as follows. A bacterial artificial chromosome containing Cacng1 genomic sequence was modified such that a floxed lacZ reporter cassette containing a neomycin resistance gene under the control of the human UBC (ubiquitin) promoter replaced 224 bp of Cacng1 coding exon 1 beginning just after the start ATG. The cassette was cloned such that lacZ coding sequence was in frame with the start ATG and the 3′ 5 bp of Cacng1 coding exon 1 remain following the cassette. (See, FIG. 2) This construct was electroporated into 100% C57Bl/6NTac embryonic stem cells. Successfully targeted clones were identified by TaqMan analysis. Cacng1−/+ mice were generated using the VelociGene© method (Valenzuela 2003 Nat Biotech PMID:12730667; Poueymirou 2007 Nat Biotech PMID:17187059) and bred to homozygosity (CACNG1−/−) as needed. The resistance cassette was removed in the F0 germline using self-deleting technology.

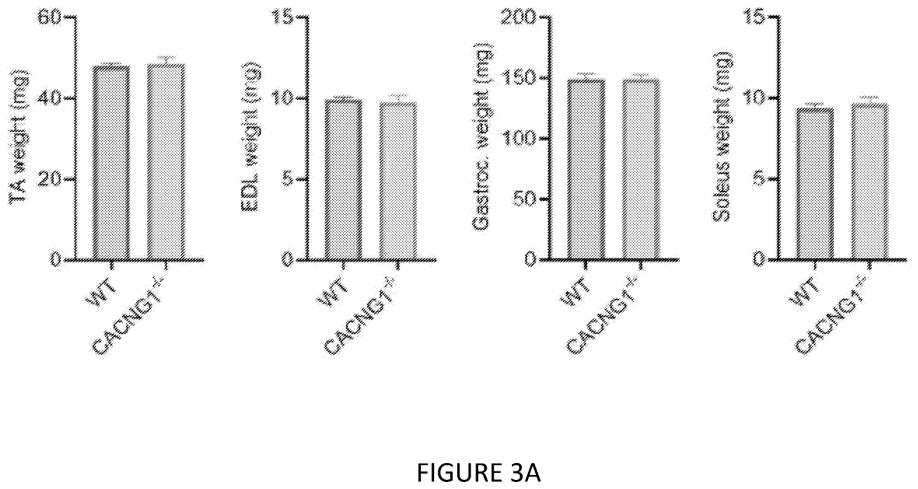

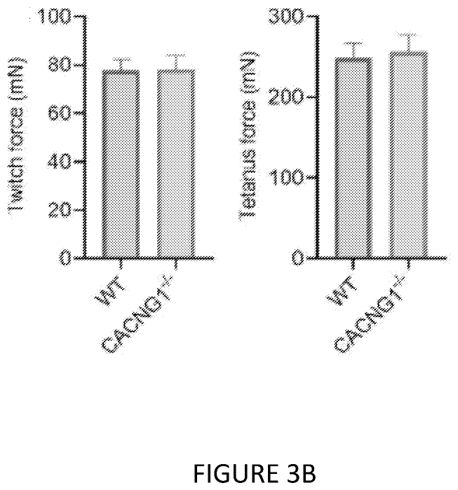

Muscle tissue from adult (5-7 months old) male WT and CACNG1^{-/-} mice were carefully dissected and weighed to assess muscle mass, and ex vivo contractility measures of isolated extensor digitorum longus (EDL) muscles was performed to assess muscle function. Muscle contractility measures were performed using an Aurora Scientific 1300A apparatus. EDL muscles were carefully excised and attached to the muscle physiology device via sutures. Muscles were then equilibrated at optimal length in Krebs-Henseleit buffer oxygenated with 95% 02/5% CO₂ and subsequently stimulated with supramaximal biphasic current to elicit a twitch response. Following twitch stimulation, a force-frequency tetanus curve was generated via stimulation at 40 Hz, 60 Hz, 80 Hz, 100 Hz, and 125 Hz with 2 minutes rest between stimuli. Maximal force production was recorded at 100 Hz. CACNG1 does not appear to play a major role in regulating muscle function, as the twitch force and tetanus force were similar between WT and CACNG^{-/-} mice. See FIGS. 3A and 3B.

### Example 3. Generation and Analysis of CACNG1 Humanized Mice (CACNG1^{hu/hu})

The Cacng1 targeting construct was designed as follows. A bacterial artificial chromosome containing the complete mouse Cacng1 genomic sequence was modified to humanize the Cacng1 locus. Part of coding exon 1, intron 1, coding exons 2-4 (and intervening introns), and 82 bp of 3′ untranslated region (UTR) mouse Cacng1 were replaced with human CACNG1 sequence consisting of coding exon 1 sequence minus the first 15 bp (this start sequence remains mouse), intron 1, coding exons 2-4 (and intervening introns), complete 3′ UTR and an additional 158 bp after the 3′ UTR of human CACNG1. See FIGS. 2A-2C. A self-deleting neomycin resistance cassette was inserted downstream of the human sequence, with the remainder of the mouse 3′ UTR to follow. See FIGS. 2A-2C, illustrating the target site after deletion of the self-deleting neomycin resistance cassette. This targeting vector was then electroporated into a 50% C57Bl/6NTac/50% 129SvEvTac embryonic stem cell line. Successfully targeted clones were identified by TaqMan analysis. Cacng1+/+ mice were generated using the VelociGene© method (Valenzuela 2003 Nat Biotech PMID:12730667; Poueymirou 2007 Nat Biotech PMID:17187059) and backcrossed to C57Bl/6NTac as needed. Antibiotic resistance cassettes were removed in the F0 male germline using self-deleting technology.

### Gene Expression Analysis

Total RNA was isolated from tissues via TRIzol homogenization and chloroform phase separation, followed by purification with MagMAX-96 for Microarrays Total RNA Isolation Kit. Genomic DNA was removed using RNAse-Free DNAse Set, and mRNA was reverse transcribed into cDNA using SuperScript VILO Master Mix. cDNA was amplified with the SensiFAST Probe Lo-Rox using the 12K Flex System. Taqman gene expression assays were used to determine human (h) and mouse (m) CACNG1 expression relative to m18S (endogenous control), and data were analyzed using the comparative CT method (ΔΔCt). Taqman primer/probe sequences were as follows: hCACNG1: fwd—GGCGAGAGCTCGGAGATC (SEQ ID NO:30), rev—GGCTGCCCAGGATGATGAAG (SEQ ID NO:31), probe—TCGAATTCACCACTCAGAAGGAGTACA (SEQ ID NO:32); mCACNG1: fwd—CCGTGCACAACAAAGACAAGAG (SEQ ID NO:33), rev—GCTCTCCCCTGGGTTGAAG (SEQ ID NO:34), probe—TGTGAGCACGTCACACCATCAGG (SEQ ID NO:35). FIG. 3A are graphs demonstrating that the expression of mouse CACNG1 (mCACNG1) is not detectable by qPCR in CACNG1^{hu/hu} mouse muscle (left graph), while human CACNG1 (hCACNG1) is expressed in CACNG1^{hu/hu}, but not WT mouse muscle (right graph).

### Single Myofiber Isolation and Live Staining

Single myofibers were isolated from the gastrocnemius muscle of adult male CACNG1^{hu/hu} mice. Muscle was carefully excised and digested with 700 U/mL collagenase in DMEM for 60 minutes. Single myofibers were isolated with a flame-polished glass Pasteur pipette, and after several rounds of digestion and washing, myofibers were plated overnight in low-adherence tissue culture plates. The next morning, human-specific, Alexa 647-conjugated CACNG1 antibodies were added to live single myofibers at 100 nM concentration for either 30 minutes or 4 hours. Myofibers were then washed with DMEM, fixed in 4% PFA, and stained for DAPI. Single fibers were then transferred to microscope slides, mounted with Fluoromount and imaged with an LSM880 confocal microscope. See FIG. 3B. The experiment demonstrates live staining of single skeletal myofibers with 100 nM of Alexa 647-conjugated human-specific α-CACNG1 Ab showing binding to myofibers isolated from CACNG1^{hu/hu} mice, but not to myofibers isolated from WT mice.

### Cryo-Fluorescence Tomography (CryoFT) Imaging of Antibody Distribution

Adult male CACNG1^{hu/hu} mice were tail vein injected with 10 mg/kg of human-specific, Alexa 647-conjugated CACNG1 antibody, Alexa 647-conjugated isotype control antibody, or saline. Six days following injection, mice were euthanized via CO₂, frozen whole, and were assessed using CryoFT processing and imaging. See FIG. 3C. The images of CACNG1^{hu/hu} mice injected with 10 mg/kg Alexa 647-conjugated human-specific α-CACNG1 Ab show high specificity for skeletal muscle compared to isotype control Ab 6 days following injection.

### Immunofluorescent Imaging of Antibody Distribution

Adult male CACNG1^{hu/hu} mice were subcutaneously injected with 10 mg/kg of human-specific, Alexa 647-conjugated CACNG1 antibody, Alexa 647-conjugated isotype control antibody, or saline. Six days following injection, mice were transcardially perfused with PBS, and the gastrocnemius/plantaris/soleus muscle complex was submerged in OCT embedding medium and frozen in liquid nitrogen-cooled isopentane. Tissues were cryosectioned at 12 μm thickness and subsequently fixed with 4% PFA and stained for laminin and DAPI. Slides were mounted with Fluoromount and imaged with an Axioscan slide scanner. See FIG. 3D. The top panel displays an endogenous Alexa 647 signal from Abs that were injected in vivo and bottom panel displays an overlay of Alexa647-Ab binding with laminin and DAPI co-staining to visualize muscle morphology.

We claim:

1. A non-human animal cell, wherein the non-human animal cell comprises a nucleic acid sequence encoding a heterologous Calcium Voltage-Gated Channel Auxiliary Subunit Gamma 1 (CACNG1) protein or a portion thereof.

2. The non-human animal cell of claim 1, wherein the nucleic acid sequence encoding the heterologous CACNG1 protein or the portion thereof comprises:
(i) a nucleic acid sequence comprising exon 1 of a human CACNG1 gene or a portion thereof;
(ii) a nucleic acid sequence comprising exon 2 of a human CACNG1 gene or a portion thereof;
(iii) a nucleic acid sequence comprising exon 3 of a human CACNG1 gene or a portion thereof;
(iv) a nucleic acid sequence comprising exon 4 of a human CACNG1 gene or a portion thereof; or
(v) any combination of (i)-(iv).

3. The non-human animal cell of claim 1 or claim 2, wherein the nucleic acid sequence encoding the heterologous CACNG1 protein or the portion thereof comprises:
(i) a nucleic acid sequence comprising exon 1 of a human CACNG1 gene or a portion thereof;
(ii) a nucleic acid sequence comprising intron 1 of a human CACNG1 gene or a portion thereof;
(iii) a nucleic acid sequence comprising exon 2 of a human CACNG1 gene or a portion thereof;
(iv) a nucleic acid sequence comprising intron 2 of a human CACNG1 gene or a portion thereof;
(v) a nucleic acid sequence comprising exon 3 of a human CACNG1 gene or a portion thereof;
(vi) a nucleic acid sequence comprising intron 3 of a human CACNG1 gene or a portion thereof;
(vii) a nucleic acid sequence comprising exon 4 of a human CACNG1 gene or a portion thereof;
(viii) a nucleic acid sequence comprising a 3′ untranslated region (UTR) of a human CACNG1 gene; or
(ix) any combination of (i)-(viii).

4. The non-human animal cell of any one of claims 1-3, wherein the nucleic acid sequence encoding the heterologous CACNG1 protein or the portion thereof comprises, consists essentially of, or consists of a nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28.

5. The non-human animal cell of any one of claims 1-4, wherein the nucleic acid sequence encoding the heterologous CACNG1 protein or portion thereof is at an endogenous Cacng1 locus.

6. The non-human animal cell of any one of claims 1-5, wherein the nucleic acid sequence encoding the heterologous CACNG1 protein or portion thereof replaces an orthologous endogenous nucleic acid sequence encoding an endogenous CACNG1 protein or a portion thereof.

7. The non-human animal cell of any one of claims 1-6, wherein the non-human animal cell, comprises an endogenous Cacng1 locus, and wherein the endogenous Cacng1 locus comprises a heterozygous or homozygous replacement of an endogenous nucleic acid sequence encoding an endogenous CACNG1 protein or a portion thereof with the nucleic acid sequence encoding the heterologous CACNG1 protein or the portion thereof,
wherein the endogenous nucleic acid sequence encoding the endogenous CACNG1 protein or the portion thereof and the nucleic acid sequence encoding the heterologous CACNG1 protein or the portion thereof are orthologous.

8. The non-human animal cell of any one of claims 1-8, wherein the heterologous CACNG1 protein or the portion thereof comprises an amino acid sequence of a human CACNG1 protein or a portion thereof.

9. The non-human animal cell of any one of claims 1-8, wherein the heterologous CACNG1 protein or the portion thereof comprises
(i) an amino acid sequence set forth as SEQ ID NO:8;
(ii) an amino acid sequence set forth as SEQ ID NO: 10;
(iii) an amino acid sequence set forth as SEQ ID NO: 12;
(iv) an amino acid sequence set forth as SEQ ID NO: 14;
(v) an amino acid sequence set forth as SEQ ID NO: 16;
(vi) an amino acid sequence set forth as SEQ ID NO:18;
(vii) an amino acid sequence set forth as SEQ ID NO:20;
(viii) an amino acid sequence set forth as SEQ ID NO:22;
(ix) an amino acid sequence set forth as SEQ ID NO:24; or
(x) any combination of (i)-(ix).

10. The non-human animal cell of any one of claims 1-3 or 5-9, wherein the heterologous CACNG1 protein or the portion thereof comprises an amino acid sequence set forth as SEQ ID NO: 4.

11. The non-human animal cell of any one of claims 1-10, wherein the non-human animal cell is a mammalian cell.

12. The non-human animal cell of any one of claims 1-11, wherein the mammalian cell is a rodent cell.

13. The non-human animal cell of any one of claims 1-12, wherein the rodent cell is a rat cell or a mouse cell.

14. The non-human animal cell of any one of claims 1-13, wherein the heterologous CACNG1 protein is a full-length human CACNG1 protein, and wherein the non-human animal cell expresses, on its cell surface, the full-length human CACNG1 protein.

15. The non-human animal cell of any one of claims 1-14, wherein the non-human animal cell is a non-human animal skeletal muscle cell that expresses, on its cell surface, the heterologous CACNG1 protein or portion thereof.

16. The non-human animal cell of any one of claims 1-13, wherein the non-human animal cell is a non-human animal cell that does not express, on its cell surface, the heterologous CACNG1 protein or portion thereof.

17. The non-human animal cell of any one of claims 1-13 and 16, wherein the non-human animal cell does not express, on its cell surface, the heterologous CACNG1 protein or the portion thereof, and wherein the non-human animal cell is not a skeletal cell.

18. The non-human animal cell of any one of claims 1-13 and 16-17, wherein the non-human animal cell does not express, on its cell surface, the heterologous CACNG1 protein or portion thereof, and wherein the non-human animal cell is a pluripotent cell.

19. The non-human animal cell of any one of claims 1-13 and 16-18, wherein the non-human animal cell does not express, on its cell surface, the heterologous CACNG1 protein or portion thereof, and wherein the non-human animal cell is an embryonic stem cell.

20. The non-human animal cell of any one of claims 1-13 and 16-18, wherein the non-human animal cell does not express, on its cell surface, the heterologous CACNG1 protein or portion thereof, and wherein the non-human animal cell is a germ cell.

21. The non-human animal cell of any one of claims 1-21, wherein the non-human animal cell is a mouse cell.

22. The non-human animal cell of any one of claims 1-21, wherein the non-human animal cell is a mouse cell and wherein the nucleic acid sequence encoding a heterologous CACNG1 protein or portion thereof comprises, consists essentially of, or consists of a nucleic acid sequence set forth as SEQ ID NO: 6.

23. A non-human animal comprising a nucleic acid sequence encoding a heterologous Calcium Voltage-Gated Channel Auxiliary Subunit Gamma 1 (CACNG1) protein or a portion thereof.

24. The non-human animal of claim 23, wherein the nucleic acid sequence encoding the heterologous CACNG1 protein or the portion thereof comprises:
(i) a nucleic acid sequence comprising exon 1 of a human CACNG1 gene or a portion thereof;
(ii) a nucleic acid sequence comprising exon 2 of a human CACNG1 gene or a portion thereof;
(iii) a nucleic acid sequence comprising exon 3 of a human CACNG1 gene or a portion thereof;
(iv) a nucleic acid sequence comprising exon 4 of a human CACNG1 gene or a portion thereof; or
(v) any combination of (i)-(iv).

25. The non-human animal of claim 23 or claim 24, wherein the nucleic acid sequence encoding the heterologous CACNG1 protein or the portion thereof comprises:
(i) a nucleic acid sequence comprising exon 1 of a human CACNG1 gene or a portion thereof;
(ii) a nucleic acid sequence comprising intron 1 of a human CACNG1 gene or a portion thereof;
(iii) a nucleic acid sequence comprising exon 2 of a human CACNG1 gene or a portion thereof;
(iv) a nucleic acid sequence comprising intron 2 of a human CACNG1 gene or a portion thereof;
(v) a nucleic acid sequence comprising exon 3 of a human CACNG1 gene or a portion thereof;
(vi) a nucleic acid sequence comprising intron 3 of a human CACNG1 gene or a portion thereof;
(vii) a nucleic acid sequence comprising exon 4 of a human CACNG1 gene or a portion thereof;
(viii) a nucleic acid sequence comprising a 3′ untranslated region (UTR) of a human CACNG1 gene; or
(ix) any combination of (i)-(viii).

26. The non-human animal of any one of claims 23-25, wherein the nucleic acid sequence encoding the heterologous CACNG1 protein or the portion thereof comprises, consists essentially of, or consists of a nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28.

27. The non-human animal of any one of claims 23-26, wherein the nucleic acid sequence encoding the heterologous CACNG1 protein or portion thereof is at an endogenous Cacng1 locus.

28. The non-human animal of any one of claims 23-27, wherein the nucleic acid sequence encoding the heterologous CACNG1 protein or portion thereof replaces an orthologous endogenous nucleic acid sequence encoding an endogenous CACNG1 protein or a portion thereof.

29. The non-human animal of any one of claims 23-28, wherein the non-human animal, comprises an endogenous Cacng1 locus, and wherein the endogenous Cacng1 locus comprises a heterozygous or homozygous replacement of an endogenous nucleic acid sequence encoding an endogenous CACNG1 protein or a portion thereof with the nucleic acid sequence encoding the heterologous CACNG1 protein or the portion thereof,
wherein the endogenous nucleic acid sequence encoding the endogenous CACNG1 protein or the portion thereof and the nucleic acid sequence encoding the heterologous CACNG1 protein or the portion thereof are orthologous.

30. The non-human animal of any one of claims 23-29, wherein the heterologous CACNG1 protein or the portion thereof comprises an amino acid sequence of a human CACNG1 protein or a portion thereof.

31. The non-human animal of any one of claims 23-30, wherein the heterologous CACNG1 protein or the portion thereof comprises
(i) an amino acid sequence set forth as SEQ ID NO:8;
(ii) an amino acid sequence set forth as SEQ ID NO: 10;
(iii) an amino acid sequence set forth as SEQ ID NO: 12;
(iv) an amino acid sequence set forth as SEQ ID NO: 14;
(v) an amino acid sequence set forth as SEQ ID NO: 16;
(vi) an amino acid sequence set forth as SEQ ID NO:18;
(vii) an amino acid sequence set forth as SEQ ID NO:20;
(viii) an amino acid sequence set forth as SEQ ID NO:22;
(ix) an amino acid sequence set forth as SEQ ID NO:24; or
(x) any combination of (i)-(ix).

32. The non-human animal of any one of claims 23-31, wherein the heterologous CACNG1 protein or the portion thereof comprises an amino acid sequence set forth as SEQ ID NO: 4.

33. The non-human animal of any one of claims 23-32, wherein the non-human animal is a mammal.

34. The non-human animal of any one of claims 23-33, wherein the mammal is a rodent.

35. The non-human animal of any one of claims 23-34, wherein the rodent is rat or a mouse.

36. The non-human animal of any one of claims 23-35, wherein the non-human animal comprises a non-human muscle cell that expresses, on its cell surface, a full-length human CACNG1 protein.

37. The non-human animal of any one of claims 23-36, wherein the non-human animal comprises a non-human animal skeletal muscle cell that expresses, on its cell surface, the heterologous CACNG1 protein or portion thereof.

38. The non-human animal of any one of claims 23-37, wherein the heterologous CACNG1 protein comprises a full-length human CACNG1 protein, and
wherein the non-human animal comprises a non-human animal skeletal muscle cell that expresses, on a cell surface, the full-length human CACNG1 protein.

39. The non-human animal of any one of claims 23-38, wherein the non-human animal comprises a non-human animal cell that does not express, on a cell surface, the heterologous CACNG1 protein or portion thereof.

40. The non-human animal of any one of claims 23-35 and 39, wherein the non-human animal comprises a non-human animal germ cell that does not express, on a cell surface of its skeletal cells, the heterologous CACNG1 protein or the portion thereof.

41. The non-human animal of any one of claims 23-40, wherein the non-human animal is a rat or a mouse.

42. A non-human animal genome, wherein the non-human animal genome comprises a nucleic acid sequence encoding a heterologous Calcium Voltage-Gated Channel Auxiliary Subunit Gamma 1 (CACNG1) protein or a portion thereof.

43. The non-human animal genome of claim 42, wherein the nucleic acid sequence encoding the heterologous CACNG1 protein or the portion thereof comprises:
(i) a nucleic acid sequence comprising exon 1 of a human CACNG1 gene or a portion thereof;
(ii) a nucleic acid sequence comprising exon 2 of a human CACNG1 gene or a portion thereof;
(iii) a nucleic acid sequence comprising exon 3 of a human CACNG1 gene or a portion thereof;
(iv) a nucleic acid sequence comprising exon 4 of a human CACNG1 gene or a portion thereof; or
(v) any combination of (i)-(iv).

44. The non-human animal genome of claim 42 or claim 43, wherein the nucleic acid sequence encoding the heterologous CACNG1 protein or the portion thereof comprises:
(i) a nucleic acid sequence comprising exon 1 of a human CACNG1 gene or a portion thereof;
(ii) a nucleic acid sequence comprising intron 1 of a human CACNG1 gene or a portion thereof;
(iii) a nucleic acid sequence comprising exon 2 of a human CACNG1 gene or a portion thereof;
(iv) a nucleic acid sequence comprising intron 2 of a human CACNG1 gene or a portion thereof;
(v) a nucleic acid sequence comprising exon 3 of a human CACNG1 gene or a portion thereof;
(vi) a nucleic acid sequence comprising intron 3 of a human CACNG1 gene or a portion thereof;
(vii) a nucleic acid sequence comprising exon 4 of a human CACNG1 gene or a portion thereof;
(viii) a nucleic acid sequence comprising a 3′ untranslated region (UTR) of a human CACNG1 gene; or
(ix) any combination of (i)-(viii).

45. The non-human animal genome of any one of claims 42-44, wherein the nucleic acid sequence encoding the heterologous CACNG1 protein or the portion thereof comprises, consists essentially of, or consists of a nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28.

46. The non-human animal genome of any one of claims 42-45, wherein the nucleic acid sequence encoding the heterologous CACNG1 protein or portion thereof is at an endogenous Cacng1 locus.

47. The non-human animal genome of any one of claims 42-46, wherein the nucleic acid sequence encoding the heterologous CACNG1 protein or portion thereof replaces an orthologous endogenous nucleic acid sequence encoding an endogenous CACNG1 protein or a portion thereof.

48. The non-human animal genome of any one of claims 42-47, wherein the non-human animal genome comprises an endogenous Cacng1 locus, and wherein the endogenous Cacng1 locus comprises a heterozygous or homozygous replacement of an endogenous nucleic acid sequence encoding an endogenous CACNG1 protein or a portion thereof with the nucleic acid sequence encoding the heterologous CACNG1 protein or the portion thereof,
wherein the endogenous nucleic acid sequence encoding the endogenous CACNG1 protein or the portion thereof and the nucleic acid sequence encoding the heterologous CACNG1 protein or the portion thereof are orthologous.

49. The non-human animal genome of any one of claims 42-48, wherein the heterologous CACNG1 protein or the portion thereof comprises an amino acid sequence of a human CACNG1 protein or a portion thereof.

50. The non-human animal genome of any one of claims 42-49, wherein the heterologous CACNG1 protein or the portion thereof comprises
(i) an amino acid sequence set forth as SEQ ID NO:8;
(ii) an amino acid sequence set forth as SEQ ID NO: 10;
(iii) an amino acid sequence set forth as SEQ ID NO: 12;
(iv) an amino acid sequence set forth as SEQ ID NO: 14;
(v) an amino acid sequence set forth as SEQ ID NO:16;
(vi) an amino acid sequence set forth as SEQ ID NO:18;
(vii) an amino acid sequence set forth as SEQ ID NO:20;
(viii) an amino acid sequence set forth as SEQ ID NO:22;
(ix) an amino acid sequence set forth as SEQ ID NO:24; or
(x) any combination of (i)-(ix).

51. The non-human animal genome of any one of claims 42-50, wherein the heterologous CACNG1 protein or the portion thereof comprises an amino acid sequence set forth as SEQ ID NO: 4.

52. The non-human animal genome of any one of claims 42-51, wherein the non-human genome is a mammalian nucleic acid.

53. The non-human animal genome of any one of claims 42-52, wherein the mammal is a rodent nucleic acid.

54. The non-human animal genome of any one of claims 42-53, wherein the rodent is rat genome or a mouse nucleic acid.

55. The non-human animal genome of any one of claims 42-54 wherein the the non-human animal genome is a mouse nucleic acid.

56. A chimeric nucleic acid molecule, comprising a nucleic acid sequence of a non-human animal Cacng1 gene that (a) encodes a CACNG1 protein and (b) is modified to comprise a replacement of a sequence encoding the CACNG1 protein or portion thereof with a homologous sequence encoding a heterologous CACNG1 protein or a portion thereof,
wherein the chimeric nucleic acid molecule encodes a functional CACNG1 protein.

57. The chimeric nucleic acid molecule of claim 56, wherein the chimeric nucleic acid sequence further comprises promoter and/or regulatory sequences of the non-human animal Cacng1 gene.

58. The chimeric nucleic acid molecule of claim 57, wherein the homologous nucleic acid sequence comprises:
(i) a nucleic acid sequence comprising exon 1 of a human CACNG1 gene or a portion thereof;
(ii) a nucleic acid sequence of intron 1 of a human CACNG1 gene or a portion thereof;
(iii) a nucleic acid sequence comprising exon 2 of a human CACNG1 gene or a portion thereof;
(iv) a nucleic acid sequence of intron 2 of a human CACNG1 gene or a portion thereof;
(v) a nucleic acid sequence comprising exon 3 of a human CACNG1 gene or a portion thereof;
(vi) a nucleic acid sequence of intron 3 of a human CACNG1 gene or a portion thereof;
(vii) a nucleic acid sequence comprising exon 4 of a human CACNG1 gene or a portion thereof;
(viii) a nucleic acid sequence of a 3′ untranslated region (UTR) of a human CACNG1 gene; or
(ix) any combination of (i)-(viii).

59. The chimeric nucleic acid molecule of claim 57 or claim 58, wherein the modified non-human animal Cacng1 gene further comprises a drug selection cassette.

60. The chimeric nucleic acid molecule of any one of claims 57-59, further comprising:
(i) a 5′ homology arm upstream of the modified non-human animal Cacng1 gene; and
(ii) a 3′ homology arm downstream of the modified non-human animal Cacng1 gene.

61. The chimeric nucleic acid of claim 60, wherein the 5′ homology arm and 3′ homology arm undergo homologous recombination with a non-human animal Cacng1 locus of interest, and
wherein following homologous recombination with the non-human animal Cacng1 locus of interest, the modified non-human animal Cacng1 gene replaces the non-human animal Cacng1 gene at the non-human animal Cacng1 locus of interest and is operably linked to an endogenous promoter that drives expression of the non-human animal Cacng1 gene at the non-human animal Cacng1 locus of interest.

62. The chimeric nucleic acid of claim 60 or claim 61, wherein:
(i) the 5′ homology arm comprises a nucleic acid sequence set forth as SEQ ID NO: 25; or
(ii) the 3′ homology arm comprises a nucleic acid sequence set forth as SEQ ID NO:26.

63. The chimeric nucleic acid molecule of any one of claims 57-62, wherein the nucleic acid sequence of the chimeric nucleic acid comprises a nucleic acid sequence set forth as SEQ ID NO:6.

64. A method of making the non-human animal cell of any one of claims 1-22, comprising inserting the nucleic acid sequence encoding the heterologous CACNG1 protein or the portion thereof into the genome of the non-human animal cell.

65. The method of claim 64, wherein the non-human animal cell is a non-human animal embryonic stem (ES) cell, and
wherein the inserting comprises inserting the nucleic acid sequence encoding the heterologous CACNG1 protein or the portion thereof into the genome of the non-human animal ES cell to form a modified non-human animal ES cell comprising the nucleic acid sequence encoding the heterologous CACNG1 protein or the portion thereof in the genome of the non-human animal ES cell.

66. The method of claim 64 or claim 65, wherein the nucleic acid sequence encoding the heterologous CACNG1 protein or the portion thereof is inserted into an endogenous Cacng1 locus.

67. The method of any one of claims 64-66, wherein the step of inserting comprises replacing an endogenous nucleic sequence encoding an endogenous CACNG1 protein or portion thereof with the nucleic acid sequence encoding the heterologous CACNG1 protein or portion thereof,
wherein the endogenous nucleic sequence encoding an endogenous CACNG1 protein or portion thereof and the nucleic acid sequence encoding the heterologous CACNG1 protein or portion thereof are orthologous.

68. The method of any one of claims 64-67, wherein the nucleic acid sequence encoding a heterologous CACNG1 protein or portion thereof comprises:
(i) a nucleic acid sequence comprising exon 1 of a human CACNG1 gene or a portion thereof;
(ii) a nucleic acid sequence comprising exon 2 of a human CACNG1 gene or a portion thereof;
(iii) a nucleic acid sequence comprising exon 3 of a human CACNG1 gene or a portion thereof;
(iv) a nucleic acid sequence comprising exon 4 of a human CACNG1 gene or a portion thereof; or
(v) any combination of (i)-(iv).

69. The method of any one of claims 64-68, wherein the nucleic acid sequence encoding a heterologous CACNG1 protein or portion thereof comprises:
(i) a nucleic acid sequence comprising exon 1 of a human CACNG1 gene or a portion thereof;
(ii) a nucleic acid sequence of intron 1 of a human CACNG1 gene or a portion thereof;
(iii) a nucleic acid sequence comprising exon 2 of a human CACNG1 gene or a portion thereof;
(iv) a nucleic acid sequence of intron 2 of a human CACNG1 gene or a portion thereof;
(v) a nucleic acid sequence comprising exon 3 of a human CACNG1 gene or a portion thereof;
(vi) a nucleic acid sequence of intron 3 of a human CACNG1 gene or a portion thereof;
(vii) a nucleic acid sequence comprising exon 4 of a human CACNG1 gene or a portion thereof;
(viii) a nucleic acid sequence of a 3′ untranslated region (UTR) of a human CACNG1 gene; or
(ix) any combination of (i)-(viii).

70. The method of any one of claims 64-69, wherein the nucleic acid sequence encoding a heterologous CACNG1 protein or portion thereof comprises, consists essentially of, or consists of a nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28.

71. The method of any one of claims 64-70, wherein the heterologous CACNG1 protein or portion thereof comprises an amino acid sequence of a human CACNG1 protein or portion thereof.

72. The method of any one of claims 64-71, wherein the heterologous CACNG1 protein or portion thereof comprises
(i) an amino acid sequence set forth as SEQ ID NO:8;
(ii) an amino acid sequence set forth as SEQ ID NO:10;
(iii) an amino acid sequence set forth as SEQ ID NO:12;
(iv) an amino acid sequence set forth as SEQ ID NO:14;
(v) an amino acid sequence set forth as SEQ ID NO:16;
(vi) an amino acid sequence set forth as SEQ ID NO:18;
(vii) an amino acid sequence set forth as SEQ ID NO:20;
(viii) an amino acid sequence set forth as SEQ ID NO:22;
(ix) an amino acid sequence set forth as SEQ ID NO:24; or
(x) any combination of (i)-(ix).

73. The method of any one of claims 64-72, wherein the heterologous CACNG1 protein comprises an amino acid sequence set forth as SEQ ID NO: 4.

74. The method of any one of claims 64-73, wherein the non-human animal cell is a mammalian cell.

75. The method of any one of claims 64-74, wherein the mammalian cell is a rodent cell.

76. The method of any one of claims 64-75, the rodent cell is a mouse cell or a rat cell.

77. The method of any one of claims 64-76, wherein the non-human animal cell is a mouse cell and wherein the nucleic acid sequence encoding a heterologous CACNG1 protein or portion thereof comprises, consists essentially of, or consists of a nucleic acid sequence set forth as SEQ ID NO: 6.

78. The method of any one of claims 64-77, wherein inserting comprises contacting the genome of the non-human animal cell with the chimeric nucleic acid molecule of any one of claims 55-63.

79. The method of any one of claims 64-78, wherein inserting comprises contacting the genome of the non-human animal cell with the chimeric nucleic acid molecule of any one of claims 56-63.

80. A method of making the non-human animal of any one of claims 23-41, comprising inserting a nucleic acid sequence encoding the heterologous CACNG1 protein or the portion thereof into the genome of a non-human animal embryonic stem (ES) cell to form a modified non-human animal ES cell comprising the nucleic acid sequence encoding the heterologous CACNG1 protein or the portion thereof in the genome of the non-human animal ES cell;
introducing the non-human animal ES cells into host embryo cells in vitro; and
gestating, in a non-human surrogate mother animal, the host embryo cells comprising the modified non-human animal ES cell, and wherein, after the gestating, the non-human surrogate mother animal births a non-human animal progeny comprising a germ cell comprising the nucleic acid sequence encoding the heterologous CACNG1 protein or portion thereof.

81. The method of claim 80, wherein the nucleic acid sequence encoding the heterologous CACNG1 protein or the portion thereof is inserted into an endogenous Cacng1 locus.

82. The method of claim 80 or claim 81, wherein the step of inserting comprises replacing an endogenous nucleic sequence encoding an endogenous CACNG1 protein or portion thereof with the nucleic acid sequence encoding the heterologous CACNG1 protein or portion thereof,
wherein the endogenous nucleic sequence encoding an endogenous CACNG1 protein or portion thereof and the nucleic acid sequence encoding the heterologous CACNG1 protein or portion thereof are orthologous.

83. The method of any one of claims 80-82, wherein the nucleic acid sequence encoding a heterologous CACNG1 protein or portion thereof comprises:
(i) a nucleic acid sequence comprising exon 1 of a human CACNG1 gene or a portion thereof;
(ii) a nucleic acid sequence comprising exon 2 of a human CACNG1 gene or a portion thereof;
(iii) a nucleic acid sequence comprising exon 3 of a human CACNG1 gene or a portion thereof;
(iv) a nucleic acid sequence comprising exon 4 of a human CACNG1 gene or a portion thereof; or
(v) any combination of (i)-(iv).

84. The method of any one of claims 80-83, wherein the nucleic acid sequence encoding a heterologous CACNG1 protein or portion thereof comprises:
(i) a nucleic acid sequence comprising exon 1 of a human CACNG1 gene or a portion thereof;
(ii) a nucleic acid sequence of intron 1 of a human CACNG1 gene or a portion thereof;
(iii) a nucleic acid sequence comprising exon 2 of a human CACNG1 gene or a portion thereof;
(iv) a nucleic acid sequence of intron 2 of a human CACNG1 gene or a portion thereof;
(v) a nucleic acid sequence comprising exon 3 of a human CACNG1 gene or a portion thereof;
(vi) a nucleic acid sequence of intron 3 of a human CACNG1 gene or a portion thereof;
(vii) a nucleic acid sequence comprising exon 4 of a human CACNG1 gene or a portion thereof;
(viii) a nucleic acid sequence of a 3′ untranslated region (UTR) of a human CACNG1 gene; or
(ix) any combination of (i)-(viii).

85. The method of any one of claims 80-84, wherein the nucleic acid sequence encoding a heterologous CACNG1 protein or portion thereof comprises, consists essentially of, or consists of a nucleic acid sequence selected from the group consising of a nucleic acid sequence set forth as SEQ ID NO:5, a nucleic acid sequence set forth as SEQ ID NO:27, and a nucleic acid sequence set forth as SEQ ID NO:28.

86. The method of any one of claims 80-85, wherein the heterologous CACNG1 protein or portion thereof comprises an amino acid sequence of a human CACNG1 protein or portion thereof.

87. The method of any one of claims 80-86, wherein the heterologous CACNG1 protein or portion thereof comprises
(i) an amino acid sequence set forth as SEQ ID NO:8;
(ii) an amino acid sequence set forth as SEQ ID NO:10;
(iii) an amino acid sequence set forth as SEQ ID NO:12;
(iv) an amino acid sequence set forth as SEQ ID NO:14;
(v) an amino acid sequence set forth as SEQ ID NO:16;
(vi) an amino acid sequence set forth as SEQ ID NO:18;
(vii) an amino acid sequence set forth as SEQ ID NO:20;
(viii) an amino acid sequence set forth as SEQ ID NO:22;
(ix) an amino acid sequence set forth as SEQ ID NO:24; or
(x) any combination of (i)-(ix).

88. The method of any one of claims 80-87, wherein the heterologous CACNG1 protein comprises an amino acid sequence set forth as SEQ ID NO: 4.

89. The method of any one of claims 80-88, wherein the non-human animal is a mammal.

90. The method of any one of claims 80-89, wherein the mammal is a rodent.

91. The method of any one of claims 80-90, the rodent cell is a mouse cell or a rat cell.

92. The method of any one of claims 80-91, wherein the non-human animal is a mouse and wherein the nucleic acid sequence encoding a heterologous CACNG1 protein or portion thereof comprises, consists essentially of, or consists of a nucleic acid sequence set forth as SEQ ID NO: 6.

93. The non-human animal of any one of claims 23-40, or the non-human animal made according to the method of any one of claims 80-92, comprising an antigen-binding protein that binds the heterologous CACNG1 protein, wherein the non-human animal expresses the heterologous CACNG1 protein or an extracellular domain thereof on a surface of a skeletal muscle cell.

94. The non-human animal of claim 93, wherein the heterologous CACNG1 protein is a human CACNG1 protein.

95. The non-human animal of claim 93 or claim 94, wherein the non-human animal is a mouse.

96. A non-human animal, non-human animal cell, or non-human animal genome comprising a knockout mutation of an endogenous Cacng1 gene.

97. The non-human animal, non-human animal cell, or non-human animal genome of claim 96, wherein the knockout mutation comprises a deletion of the Cacng1 gene or a portion thereof.

98. The non-human animal, non-human animal cell, or non-human animal genome of claim 96 or claim 97, wherein the knockout mutation comprises a deletion of the entire coding sequence of the Cacng1 gene.

99. The non-human animal, non-human animal cell, or non-human animal genome of claim any one of claims 96-98, wherein the non-human animal, non-human animal cell, or non-human animal genome does not express any CACNG1 protein.

100. The non-human animal, non-human animal cell, or non-human animal genome of claim any one of claims 96-99, wherein the non-human animal, non-human animal cell, or non-human animal genome does not exhibit any gross mutant phenotype.

101. The non-human animal, non-human animal cell, or non-human animal genome of anyone of claims 96-100, wherein the non-human animal, non-human animal cell, or non-human animal genome comprises an endogenous Cacng1 locus comprising the sequence set forth as SEQ ID NO:29.

102. A method of making a CACNG1 knockout non-human animal comprising modifying an endogenous Cacng1 locus of the non-human animal to comprise a knockout mutation.

103. A targeting vector comprising:
(i) a 5′ homology arm; and
(ii) a 3′ homology arm,
wherein the 5′ homology arm and 3′ homology arm undergo homologous recombination with a non-human animal Cacng1 locus of interest, and
wherein following homologous recombination with the non-human animal Cacng1 locus of interest, the targeting vector inserts a knockout mutation in the non-human animal Cacng1 gene at the non-human animal Cacng1 locus of interest.

104. A non-human animal, non-human animal cell, or non-human animal genome made according to any method described herein.
